(19) United States
(12) Patent Application Publication
(10) Pub. No.: US 2014/0364434 A1
(43) Pub. Date: Dec. 11, 2014

(54) Biomarkers for Prediction of Response to PARP Inhibition in Breast Cancer

(71) Applicant: The Regents of the University of Califonia, Oakland, CA (US)

(72) Inventors: Anneleen Daeman, Pinole, CA (US); Denise M. Wolf, Berkeley, CA (US); Laura J. Van 't Veer, San Francisco, CA (US); Paul T. Spellman, Portland, OR (US); Joe W. Gray, Lake Oswego, OR (US)

(73) Assignee: THE REGENTS OF THE UNIVERSITY OF CALIFORNIA, Oakland, CA (US)

(21) Appl. No.: 14/298,849

(22) Filed: Jun. 6, 2014

Related U.S. Application Data
(63) Continuation of application No. PCT/US2012/068622, filed on Dec. 7, 2012.
(60) Provisional application No. 61/568,146, filed on Dec. 7, 2011.
(60) Provisional application No. 61/666,671, filed on Jun. 29, 2012.

(51) Int. Cl. C12Q 1/68

(52) U.S. Cl. CPC C12Q 1/6886; C12Q 2600/106; C12Q 2600/158
USPC 514/248; 506/8

(57) ABSTRACT

Methods and systems for identifying a cancer patient suitable for treatment with a PARP inhibitor. A 6-gene, 7-gene and 8-gene predictor panels of genes that are predictive of patient resistance or sensitivity to PARP inhibitors such as Olaparib.

### CROSS-REFERENCE TO RELATED APPLICATIONS

This application is a non-provisional continuation application of and claims priority to International Patent Application No. PCT/US2012/068622, filed on Dec. 7, 2012, which claims priority to U.S. Provisional Patent Application No. 61/568,146, filed on Dec. 7, 2011, to U.S. Provisional Patent Application No. 61/666,671, filed on Jun. 29, 2012, the contents of all of which are hereby incorporated by reference.

### STATEMENT OF GOVERNMENTAL SUPPORT

The invention was made with government support under Contract No. DE-AC02-05CH11231 awarded by the U.S. Department of Energy, and under UCSF Breast SPORE Bioinformatics Grant awarded by the National Cancer Institute/National Instituted of Health. The government has certain rights in the invention.

### REFERENCE TO A SEQUENCE LISTING SUBMITTED AS A TEXT FILE VIA EFS-WEB AND TABLES

The official copy of the sequence listing is submitted concurrently with the specification as a text file via EFS-Web, in compliance with the American Standard Code for Information Interchange (ASCII), with a file name of “JIB3095US_seqlisting_ST25.txt”, a creation date of Jun. 6, 2014, and a size of 275 KB. The sequence listing filed via EFS-Web is part of the specification and is hereby incorporated in its entirety by reference herein.

Tables 1-15 in the attached Appendix to the Specification are also part of the specification and hereby incorporated by reference in their entirety.

### BACKGROUND OF THE INVENTION

1. Field of the Invention

The invention relates to the field of diagnostic and prognostic methods and applications for directing therapies of human cancers, especially breast cancer.

2. Related Art

Poly (ADP-ribose) polymerase (PARP) is an enzyme involved in DNA repair. PARP inhibitors operate on the principle of synthetic lethality in conjunction with DNA damaging agents, and are likely to be useful for treatment of BRCA-mutated cancers and triple negative breast cancers exhibiting ‘BRCA-ness’ or other signs of DNA repair deficiency. Multiple PARP inhibitors have been developed, such as Olaparib (AstraZeneca), BSI-201 (Sanofi-Aventis) and ABT-888 (Abbott Laboratories). Though some clinical trials have shown drugs in this class to be promising, not all results have been positive. As PARP inhibitors differ in mechanism of action, dosing interval and toxicities, trial results seem to depend on the specific combination of PARP inhibitor and patient population. To understand why some studies succeeded and others failed and to guide new clinical trials in patient selection, there is an urgent need for biomarker identification, both for PARP inhibitors in general and for the specific idiosyncratic mechanisms of each drug. PARP inhibitors have been incorporated into the adaptive neo-adjuvant clinical trial I-SPY2 for women with locally advanced primary breast cancer. This trial will be used to test and refine cell line based predictors of response to PARP inhibitors and other investigational agents.

In an upregulated homologous recombination (HR) pathway in HR competent cells to compensate for loss of base excision repair, double-strand breaks (DSBs) can be repaired resulting in cell survival; however, this is not the case in BRCA- or HR-deficient cells. As cells cannot use the HR pathway, DSBs are repaired via the less accurate non-homologous end joining (NHEJ) pathway or the single strand annealing subpathway of HR, resulting in large numbers of chromatid aberrations that usually lead to cell death. These conditions therefore make cells with BRCA mutations or other HR defects preferentially sensitive to (i.e. to show synthetic lethality with) PARP inhibitors.

After the interaction between BRCA1/2 and PARP1 was discovered, multiple PARP inhibitors were developed [Rouleau M, Patel A, Hendzel M J, Kaufmann S H, Poirier G G: PARP inhibition: PARP1 and beyond. Nature reviews Cancer 2010, 10(4):293-301 Vinayak S, Ford J: PARP inhibitors for the treatment and prevention of breast cancer. Curr Breast Cancer Rep 2010, 2:190-19]. These agents are designed to compete with the NAD+ binding site of PARP1, and can be used as a single agent based on the synthetic lethality principle or as chemo-potentiating agent after SSBs are created by common anticancer treatments such as radiotherapy [. Rouleau M, Patel A, Hendzel M J, Kaufmann S H, Poirier G G: PARP inhibition: PARP1 and beyond. Nature reviews Cancer 2010, 10(4):293-301Plummer R: Poly(ADP-ribose) polymerase inhibition: a new direction for BRCA and triple-negative breast cancer? Breast cancer research: BCR 2011, 13(4):218]. PARP inhibitors in clinical studies for breast cancer are Olaparib (AstraZeneca, London), BSI-201 (also known as Iniparib, BiPar Sciences Inc., Sanofi-Aventis, Paris), ABT-888 (also known as Veliparib, Abbott Laboratories, IL), PF-01367338 (also known as AG014699; Pfizer Inc., NY) and MK-4827 (Merck & Co Inc., NJ). These PARP inhibitors differ significantly in mechanism of action (reversible or irreversible inhibition), target (PARP1 or PARP1/2), dosing interval (continuous or intermittent) and toxicities [Vinayak S, Ford J: PARP inhibitors for the treatment and prevention of breast cancer. Curr Breast Cancer Rep 2010, 2:190-197]. BSI-201 differs from Olaparib, ABT-888 and PF-01367338 in both dosing interval and mechanism of action. BSI-201 is dosed intermittently and is an irreversible PARP inhibitor due to covalent bond formation. Furthermore, whilst Olaparib and ABT-888 are oral inhibitors of both PARP1 and PARP2, BSI-201 and PF-01367338 are intravenous PARP1 inhibitors.

PARP inhibitors have been proposed as possibly useful for treatment of BRCA-mutated cancers and triple negative breast cancers exhibiting ‘BRCA-ness’ [Farmer H, McCabe N, Lord C J, Tutt A N, Johnson D A, Richardson T B, Santarosa M, Dillon K J, Hickson I, Knights C et al: Targeting the DNA repair defect in BRCA mutant cells as a therapeutic strategy. Nature 2005, 434(7035):917-921, Turner N, Tutt A, Ashworth A: Hallmarks of ‘BRCAness’ in sporadic cancers. Nature reviews Cancer 2004, 4(10):814-819]. BRCA-ness is defined as the spectrum of phenotypes that some sporadic tumors share with familial-BRCA cancers, reflecting the underlying distinctive DNA-repair defect arising from loss of HR; for example, by epigenomic downregulation of BRCA1 and FANCF [Turner N, Tutt A, Ashworth A: Hallmarks of ‘BRCAness’ in sporadic cancers. Nature reviews Cancer 2004, 4(10):814-819]. PARP inhibitors in clinical studies for BRCA-associated, triple negative and/or basal-like breast cancer include olaparib (AstraZeneca, London), BSI-201, ABT-888 (also known as Veliparib; Abbott Laboratories, IL) and PF-01367338 (AG014699; Pfizer Inc., NY) and MK-4827 [13,16,17]. The majority of the studies are in Olaparib and BSI-201, although more recently the focus broadened to ABT-888, PF-01367338 and MK-4827 as well [Liang H, Tan A: PARP inhibitors. Curr Breast Cancer Rep 2011, 3:44-54]. These agents are licensed for monotherapy in DNA repair deficient patients or as chemo-potentiating agents after SSBs are created by common anticancer treatments such as radiotherapy and DNA damaging agents. For metastatic triple negative breast cancer, a phase II clinical trial of the BiPAR PARP inhibitor BSI-201 demonstrated a dramatic survival advantage when combined with gemcitabine/carboplatin chemotherapy, the likes of which has not been observed since Herceptin was introduced for ERBB2-positive cancers [O'Shaughnessy J, Osborne C, Pippen J E, Yoffe M, Patt D, Rocha C, Koo I C, Sherman B M, Bradley C: Iniparib plus chemotherapy in metastatic triple-negative breast cancer. The New England journal of medicine 2011, 364(3):205-214]. These results on metastatic triple negative breast cancer, however, could not be confirmed in a randomized, open-label phase III study [Guha M: PARP inhibitors stumble in breast cancer. Nature biotechnology 2011, 29(5):373-374, O'Shaughnessy J, Schwartzberg L, Danso M, Rugo H, Miller K, Yardley D, Carlson R, Finn R, Charpentier E, Freese M et al: A randomized phase III study of iniparib (BSI-201) in combination with gemcitabine/carboplatin (G/C) in metastatic triple-negative breast cancer (TNBC). J Clin Oncol 2011, 29:suppl; abstr 10]. Though other clinical trials have shown drugs in this class to be promising, overall not all results have been positive [Turner N C, Ashworth A: Biomarkers of PARP inhibitor sensitivity. Breast cancer research and treatment 2011, 127(1):283-286]. Results obtained from the clinical trials so far seem to highly depend on the specific breast cancer patient population, the specificity of the PARP inhibitor, and the nature of the therapeutic agent used in combination with PARP inhibitor (e.g., temozolomide, gemcitabine) [15,21]. A multicenter phase 2 trial showed that olaparib as monotherapy led to objective response rates in 41% of BRCA1/2 mutation carriers who had previously received several courses of chemotherapy [84]. Results for triple negative breast cancer patients without known BRCA1/2 mutations have been inconsistent. Preclinical studies and phase 1 trials suggested that PARP inhibitors can increase cell death in these patients when combined with paclitaxel [85], whilst triple negative breast cancer patients largely did not respond to olaparib monotherapy in a phase 2 trial [86]. Also, Olaparib and MK-4827 were efficacious when administered as single agent to hereditary BRCA1/2-related breast cancer. Also ABT-888 was efficacious in this subgroup of breast cancer when combined with DNA-damaging agent temozolomide. However, no evidence of activity was seen for the combination of ABT-888 with temozolomide in heavily pre-treated sporadic triple negative breast cancer, and negative results were obtained for the latter patient population with Olaparib as single agent. The main focus in this study is on Olaparib, a small-molecule, reversible, oral inhibitor of both PARP1 and PARP2 [Tutt A, Robson M, Garber J E, Domchek S M, Audeh M W, Weitzel J N, Friedlander M, Arun B, Loman N, Schmutzler R K, Wardley A, Mitchell G, Earl H, Wickens M, Carmichael J (2010) Oral poly(ADP-ribose) polymerase inhibitor olaparib in patients with BRCA1 or BRCA2 mutations and advanced breast cancer: a proof-of-concept trial. Lancet 376 (9737):235-244]. A phase 1 trial on Olaparib showed that only a few of the adverse effects of conventional chemotherapy are associated with Olaparib treatment and that this drug compound has antitumor activity for the majority of carriers of a BRCA1/2 mutation but not for patients without known BRCA mutations [Fong P C, Boss D S, Yap T A, Tutt A, Wu P, Mergui-Roelvink M, Mortimer P, Swaisland H, Lau A, O'Connor M J et al: Inhibition of poly(ADP-ribose) polymerase in tumors from BRCA mutation carriers. The New England journal of medicine 2009, 361(2):123-134]. Thus, identifying candidate biomarkers that can be tested for their ability to better identify subsets of sporadic cancers with defects in HR-directed repair that will respond to PARP inhibitors is needed.

### SUMMARY OF THE INVENTION

A method for predicting the response of a patient with breast cancer, said method comprising: providing breast cancer tissue from the patient; determining from the provided tissue, the level of gene amplification or gene expression for at least one of the following genes: BRCA1, BRCA2, H2AFX, MRE11A, TDG, XRCC5, CHEK1, CHEK2, MK2, NBS1 or XPA; identifying that the at least one gene or gene product is amplified; whereby, when the at least one gene or gene product is amplified, this is an indication that the patient is predicted to be sensitive or resistant to a PARP inhibitor.

Thus, a method for identifying a cancer patient suitable for treatment with a PARP inhibitor compound, comprising: (a) measuring amplification or expression levels of a gene selected from the group consisting of genes encoding BRCA1, BRCA2, H2AFX, MRE11A, TDG, XRCC5, CHEK1 and CHEK2 in a sample from the patient; and (b) comparing the amplification or expression level of the gene from the patient with amplification or expression level of the gene in a normal tissue sample or a reference expression level, wherein an increase of amplification or expression of the gene encoding BRCA2, CHEK1 or CHEK2 and/or a decrease of amplification or expression of the gene encoding BRCA1, H2AFX, MRE11A, TDG or XRCC5 indicates the patient will be suitable for treatment with the PARP inhibitor.

In some embodiments, the method for identifying a cancer patient suitable for treatment with a PARP inhibitor compound, comprising: (a) measuring amplification or expression levels of a gene selected from the group consisting of genes encoding H2AFX, MRE11A, TDG, XRCC5, CHEK1 and CHEK2 in a sample from the patient; and (b) comparing the amplification or expression level of the gene from the patient with amplification or expression level of the gene in a normal tissue sample or a reference expression level, wherein an increase of amplification or expression of the gene encoding CHEK1 or CHEK2 and/or a decrease of amplification or expression of the gene encoding H2AFX, MRE11A, TDG or XRCC5 indicates the patient will be suitable for treatment with the PARP inhibitor. In some embodiments, step (a) measuring amplification or expression levels of at least two, three, four, five or more genes selected from the group consisting of genes encoding H2AFX, MRE11A, TDG, XRCC5, CHEK1 and CHEK2 in a sample from the patient. In another embodiment, measuring amplification or expression levels of at least one gene from the resistant group (H2AFX, MRE11A, TDG or XRCC5) and one from the sensitive group (CHEK1 or CHEK2).

In some embodiments, the method for identifying a cancer patient suitable for treatment with a PARP inhibitor compound, comprising: (a) measuring amplification or expression levels of a gene selected from the group consisting of genes encoding BRCA1, MRE11A, TDG, CHEK2, MK2, NBS1 and XPA in a sample from the patient; and (b) comparing the amplification or expression level of the gene from the patient with amplification or expression level of the gene in a normal tissue sample or a reference expression level, wherein an increase of amplification or expression of the gene encoding MK2 or CHEK2 and/or a decrease of amplification or expression of the gene encoding MRE11A, TDG, BRCA1, NBS1 or XPA indicates the patient will be suitable for treatment with the PARP inhibitor. In some embodiments, step (a) measuring amplification or expression levels of at least two, three, four, five, six or more genes selected from the group consisting of genes encoding BRCA1, MRE11A, TDG, CHEK2, MK2, NBS1 and XPA in a sample from the patient. In another embodiment, measuring amplification or expression levels of at least one gene from the resistant group (BRCA1, MRE11A, TDG, NBS1 or XPA) and one from the sensitive group (MK2 or CHEK2).

Incorporating prior knowledge of DNA repair pathways and applying stringent criteria for maker inclusion using three expression platforms, herein is described a DNA repair pathway-based 8-gene diagnostic predictor panel of genes that predict response to Olaparib. This signature was observed in a substantial fraction of primary breast tumors predicted to benefit from Olaparib. About 40-49% of patients are predicted to respond to Olaparib, which was confirmed on a distinct platform. Furthermore, a higher percentage of patients expressing the 8-gene sensitivity signature are basal and ERBB2-negative.

In one embodiment, the gene predictor panel comprising an eight-gene panel comprising the following genes: BRCA1, BRCA2, CHEK1, CHEK2, H2AFX, MRE11A, TDG, and XRCC5 (Ku80).

In another embodiment, the gene predictor panel comprising a six-gene panel comprising the following genes: CHEK1, CHEK2, H2AFX, MRE11A, TDG, and XRCC5 (Ku80).

In another embodiment, the gene predictor panel comprising a seven-gene panel comprising the following genes: BRCA1, MRE11A, TDG, CHEK2, MK2, NBS1 and XPA.

### BRIEF DESCRIPTION OF THE FIGURES

FIG. 1 displays the overview of the approach used for the development of a predictor of Olaparib response in a breast cancer cell line panel with inclusion of prior knowledge of DNA repair pathways. For 22 breast cancer cell lines, growth inhibition assays were used to measure their sensitivity to Olaparib (KU0058948; KuDOS Pharmaceuticals/AstraZeneca), expressed as the surviving fraction at 50% (SF50) in μM. For these cell lines, expression data were obtained with three different platforms (Affymetrix GeneChip Human Genome U133A, Affymetrix GeneChip Human Exon 1.0 ST, and whole transcriptome shotgun sequencing (RNA-seq) measured with the Illumina GAIL The bottom-up approach was used for biomarker selection, incorporating prior knowledge of the principal DNA repair pathways BER (base excision repair), NER (nucleotide excision repair), MMR (mismatch repair), HR/FA (homologous recombination/Fanconi anemia), NHEJ (non-homologous end joining) and DDR (DNA damage response), operating at different functional levels in the cells. Biomarkers from Wang et al [2] were systematically expanded with genes assigned to any of these pathways in the Kyoto Encyclopedia of Genes and Genomes (KEGG) database release 55.1, resulting in 118 genes. For each DNA repair pathway and expression data set, logistic regression in combination with forward feature selection (5-fold CV) was then repeated 100 times to determine the most important markers selected in over half of the iterations, and further reduced to those selected with consistent pattern of sensitivity for at least 2 out of 3 platforms.

FIG. 2 provides the waterfall plot of the response to olaparib (expressed as SF50 in μM) for 22 breast cancer cell lines with molecular data, ordered from most resistant at the left to most sensitive at the right, with bars colored according to subtype (luminal in light grey, basal in black, claudin-low in dark grey, and ERBB2 amplified in white). Among those, 6 are basal with one cell line, HCC1954, ERBB2 amplified; 7 claudin-low; and 9 luminal of which 3 are ERBB2 amplified. A trend was observed towards greater sensitivity in the basal subtype and greater resistance in the luminal cell lines. The threshold of 1 μM used to divide the cell lines into a group of 15 resistant cell lines (indicated with R) and a group of 7 sensitive cell lines (indicated with S) is represented with a horizontal dashed line

FIG. 3 provides the boxplot of SF50 for the cell lines divided according to breast cancer subtype (luminal, claudin-low, basal). An association of breast cancer subtype with response to Olaparib is shown in the cell line panel, with greater sensitivity in the basal subtype and greater resistance in the luminal cell lines, although not significant due to the low number of cell lines (Kruskal-Wallis test, p-value 0.314).

FIGS. 4A and 4B show graphs which provide validation of literature markers in 22 breast cancer cell lines and an overview of individual DNA repair-associated biomarkers that are most significantly associated with drug response in the 22 breast cancer cell lines, based on copy number, expression and methylation data. Besides down-regulation of BRCA1 in the sensitive cell lines, BRCA1-mutated cell lines MDAMB436 and SUM149PT were more sensitive to Olaparib compared to the wildtype cell lines (p-value 0.037). Additionally, the sensitive cell lines were characterized by a significant lower copy number of BRCA1 (p-value 0.012). Due to the strong association in breast cancer between BRCA1 mutation and lost PTEN expression, mutation status in BRCA1 and PTEN were subsequently combined. Cell lines with a mutation in either of both genes were more sensitive to Olaparib than cell lines that were wildtype for both genes (p-value 0.051). Genes BRCA1, EMSY, ER, FANCD2, γH2AX, MRE11A, PR, TNKS2 and XRCC5 were significantly down-regulated in the sensitive compared to the resistant cell lines, according to at least one expression platform (U133A, exon array and RNA-seq). Down-regulation of ER and PR was confirmed at protein level with the reverse protein lysate array (p-value 0.126 and 0.059, respectively). Genes CHEK2, MK2, and XRCC3 were mainly up-regulated in the sensitive compared to the resistant lines.

FIG. 5 displays the heatmap of the expression of the 8 signature genes in the cell line panel: BRCA1, BRCA2, CHEK1, CHEK2, MRE11A, H2AFX, TDG and XRCC5. As expression data, gene expression measured on the Affymetrix U133A platform with use of Affymetrix's standard annotation was used. The genes were clustered with hierarchical clustering, using Euclidean distance and average linkage. The cell lines are shown from most resistant at the left to most sensitive at the right. Table 8 shows the data represented in the heatmap of FIG. 5.

FIG. 6 shows a boxplot of SF50 for the cell lines divided according to breast cancer subtype (9 luminal, 7 claudin-low, 6 basal lines). No association was found between breast cancer subtype and response to olaparib in the cell line panel (Fisher's exact test for basal vs. luminal, p-value 0.136).

FIG. 7 shows graphs which provide an overview of individual DNA repair-associated markers that are significantly associated with or do trend towards an association with response to olaparib in the 22 breast cancer cell lines, based on mutation, copy number and expression data (see Table 14 for the complete list of markers). The four boxplots at the top show the association results for BRCA1. The BRCA1-mutated cell lines MDAMB436 and SUM149PT tend to be more sensitive to olaparib compared to the wild-type cell lines (p-value 0.091). The sensitive cell lines are also characterized by a significant lower copy number of BRCA1 (p-value 0.012) and by BRCA1 down-regulation (RNA-seq, p-value 0.055). Cell lines with a deficiency in BRCA1 and/or PTEN tend to be more sensitive to olaparib than cell lines with functional BRCA1 and PTEN (p-value 0.052). The boxplots at the bottom show the association for genes NBS1 and XRCC5 that are significantly down-regulated and for genes CHEK2 and MK2 that are significantly up-regulated in the sensitive compared to the resistant cell lines.

Table 1 displays the eight genes selected for response prediction to treatment with Olaparib based on the breast cancer cell line expression data. Five of these genes are resistance markers (BRCA1, MRE11A, H2AFX, TDG and XRCC5) and three are sensitivity markers (BRCA2, CHEK1 and CHEK2). For each gene, its symbol, Entrez Gene identifier, and corresponding probe set from the Affymetrix U133A array used in the predictor are shown. A predictor for these 8 genes was obtained with the weighted voting algorithm (Moulder et al, Molecular Cancer Therapeutics 2010, 9(5):1120), using the Affymetrix U133A expression data with Affymetrix's standard annotation. The weight w_{g} and decision boundary b_{g} for each gene derived from the cell line panel are shown in this table, and can be used for the prediction of response to Olaparib in new patients, after median normalization of each gene in the patients' expression data.

Table 2 displays the set of 22 breast cancer cell lines, with response to Olaparib expressed as SF50 (μM), and availability of the different molecular data sets, indicated with 0 for unavailability and 1 for availability.

Table 3 displays the biomarkers that have been suggested as predictors for PARP inhibitor response in literature, grouped according to level of the central dogma (mutation, expression/protein level, copy number level, promoter methylation, and siRNA). The pattern of alteration that resulted in sensitivity to PARP inhibition is indicated—when clearly described in literature—with (−) corresponding to mutation, deficiency or down-regulation being associated with PARP inhibition sensitivity, and (+) indicative for up-regulation or promoter methylation resulting in sensitivity to PARP inhibition. Biomarkers grouped according to level of the central dogma. First, loss-of-function mutations in genes of the HR or DDR pathway such as BRCA1/2, ATM, ATR, PTEN, NBS1, MRE11A, CHEK1/2, and TP53 might direct to PARP inhibitor sensitivity [Wang X, Weaver D: The ups and downs of DNA repair biomarkers for PARP inhibitor therapies. Am J Cancer Res 2011, 1(3):301-327, Turner N C, Ashworth A: Biomarkers of PARP inhibitor sensitivity. Breast cancer research and treatment 2011, 127(1):283-286, Negrini S, Gorgoulis V G, Halazonetis T D: Genomic instability—an evolving hallmark of cancer. Nature reviews Molecular cell biology 2010, 11(3):220-228].

Table 4 provides an overview of the validation of the markers from literature listed in Table 3 in the set of 22 breast cancer cell lines with use of the non-parametric Wilcoxon rank sum test. Results are shown per set of markers: 4a) mutation—for genes with mutation information in the COSMIC database for the 22 breast cancer cell lines, the cell lines with a mutation in each specific gene are listed, the number of mutated cell lines, and observed response in the mutated cell lines compared to the wildtype cell lines; 4b) expression—for each gene, the significance of association of expression level with response is indicated with the p-value for all three expression platforms, with for the Affymetrix U133A array a further distinction based on the annotation file used for probe set summarization (Affymetrix's standard annotation file vs. a custom annotation file (Dai et al, Nucleic Acids Research 2005, 33(20):e175)). Moreover, the observed pattern of response in the sensitive compared to the resistant cell lines is shown, with − indicative for down-regulation of the gene in the sensitive compared to the resistant cell lines, and + for up-regulation in the sensitive compared to the resistant cell lines; 4c) copy number variation—for each gene, the copy number variation (deletion or amplification) that occurs in the sensitive cell lines compared to the resistant cell lines is shown; 4d) promoter methylation (n=22)—per gene, association of response with promoter methylation is shown for all methylation probes in the corresponding promoter region. The methylation trend in the sensitive compared to the resistant cell lines is shown, as well as the number of CG dinucleotides and number of off-CpG cytosines for each of the methylation probes; and 4e) siRNA (n=15)—for each siRNA, it is indicated whether there is less or more loss of viability in the sensitive compared to the resistant cell lines.

Table 5 provides an overview per expression platform of the genes from the 6 principal DNA repair pathways that are selected with the logistic regression approach in over half of the iterations. Biomarkers mentioned in the review paper by Wang et al (Am J Cancer Res, 2011, 1(3):301) were considered separately from genes assigned to any of the DNA repair pathways in the Kyoto Encyclopedia of Genes and Genomes (KEGG) database release 55.1. Moreover, to obtain robust markers, biomarker selection was repeated for each of the three expression platforms (Affymetrix GeneChip Human Genome U133A, Affymetrix GeneChip Human Exon 1.0 ST, and whole transcriptome shotgun sequencing (RNA-seq) measured with the Illumina GAII). For each DNA repair pathway and expression data set, logistic regression with forward selection (5-fold CV) was repeated 100 times to determine the most important markers selected in over half of the iterations. These genes selected in >250/500 iterations are displayed in this table. These markers were further reduced to those selected with consistent pattern of sensitivity for at least 2 out of 3 platforms, shown in bold. This table also displays the average 5-fold cross-validation area under the ROC curve (AUC) across the 100 randomizations for a logistic regression model with optimized logistic regression coefficients or coefficients fixed to +/−1 for sensitive and resistance markers, respectively and with the inclusion of the platform-specific genes selected in over half of the iterations.

Table 6 provides prevalence of the 8-gene signature in tumor samples. Eight U133A and two U133 plus 2 data sets on primary breast tumors with or without metastasis, heterogeneous in both treatment and ER/PR/LN status, and with number of tumor samples varying from 61 to 289 were used to verify the prevalence of the 8-gene predictor in tumor samples. Applying the 8-gene predictor obtained from the U133A cell line expression data with the weighted voting algorithm to the tumor data sets revealed that 40-49% of patients were predicted to be responsive to Olaparib. Validation in 117 tumor samples from the I-SPY1 clinical trial revealed that 41% of 1-SPY1 patients are likely to respond to Olaparib. To verify cross-platform generalizability, the signature was additionally tested in 430 breast invasive carcinoma samples collected by TCGA (The Cancer Genome Atlas) [71] for which custom Agilent 244K expression was available. Prevalence was confirmed on this distinct platform. Because genes that are consistently up-regulated in a set of cell lines should also be concurrently up-regulated in tumor samples, and similar for genes that are consistently down-regulated, we calculated the Jaccard similarity coefficient (Van Rijsbergen C: Information retrieval, Butterworth 1979). This coefficient ranges from 0 to 1 and reflects the similarity in co-expression pattern between cell lines and tumor samples. In our panel, the Jaccard coefficient was on average 0.55 with standard deviation 0.10 (min-max=[0.43 0.75]).

Table 7 displays the association of breast cancer subtype with predicted response to Olaparib in the I-SPY1 and TCGA data set. To characterize the patient population likely to respond to Olaparib according to the predictor, breast cancer subtype was associated with predicted response for 113 I-SPY1 and 422 TCGA tumor samples, after exclusion of the normal-like samples. A trend was observed towards a higher percentage of basal samples and a lower percentage of luminal B and ERBB2-amplified samples in the set of samples predicted to respond to Olaparib (p-values 0.109 and 0.014 for I-SPY1 and TCGA, respectively).

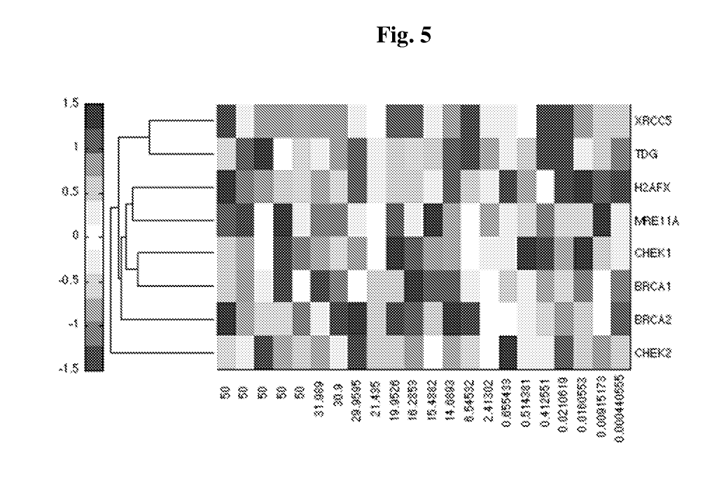

Table 8 shows the data used to generate the heatmap of FIG. 5.

Table 9 provides an overview of the breast cancer cell line panel with response to olaparib expressed as SF50 (μM); ER, PR and ERBB2 expression with + indicating up-regulation relative to the other cell lines, − down-regulation, and NC no change in expression; and availability of the different molecular data sets indicated with N for unavailability and Y for availability. Doubling times were estimated for each cell line from measurements of the number of doublings of untreated cells that occurred in 72 hours during the course of assessing responses to 123 therapeutic compounds [Heiser et al, PNAS 2012].

Table 10 provides an overview per expression platform of genes from 6 principal DNA repair pathways that are selected with the logistic regression approach in over half of the iterations

Table 11 provides an overview of the seven genes selected for prediction of response to treatment with olaparib based on breast cancer cell line expression data. The weights and decision boundaries were determined with data from the U133A expression array platform measured for the 22 cell lines used to assess response to olaparib. For each of the 5 resistance and 2 sensitivity markers, gene symbol is shown together with gene name, entrez gene identifier, corresponding probe set from the Affymetrix U133A array, and weight and decision boundary obtained with the weighted voting algorithm

Table 12 shows the prevalence of the 7-gene signature in tumor samples from 9 different studies on primary breast tumors with or without metastasis, heterogeneous in treatment and ER/PR/LN status

Table 13 shows the association of breast cancer subtype with predicted response to olaparib in 464 GSE25066 and 528 TCGA tumor samples, after exclusion of the normal-like samples

Table 14 shows the association of individual DNA repair biomarkers with response to olaparib in the breast cancer cell line panel with use of the non-parametric Wilcoxon rank sum test for continuous data (expression, copy number variation, promoter methylation) and Fisher's exact test for mutation status. Results are shown per set of markers, with significant markers (p-value<0.05) shown in bold and trending markers (0.05<p-value<0.1) in italic: 14a) expression, with for each gene the significance of association of expression with response indicated with the p-value and the fold-change (FC) with +/− indicating the direction of change in the sensitive with respect to resistant cell lines for all three expression platforms; for the Affymetrix U133A array a further distinction is made based on the annotation file used for probe set summarization; 14b) mutation, with for each gene the number of mutated cell lines among the set of sensitive and resistant lines; for BRCA1 and TP53, mutation information from the COSMIC database was used; for PTEN information on mutation status and null expression were obtained from [87] and independently validated at ICR; 14c) copy number variation, with for each gene the aberration (amplification or deletion) that occurs in the sensitive compared to the resistant cell lines; 14d) promoter methylation, with per gene the results for all methylation probes in the corresponding promoter region, with methylation trend in the sensitive compared to the resistant lines, the number of CG dinucleotides and number of off-CpG cytosines for each of the methylation probes.

Table 15 lists 118 unique DNA repair biomarkers from Wang et al, 2011 and the Kyoto Encyclopedia of Genes and Genomes (KEGG) database, divided according to the principal DNA repair pathways BER, NER, MMR, HR/FA, NHEJ and DDR

### DESCRIPTION OF THE PREFERRED EMBODIMENTS

There is increasing appreciation that response to breast cancer therapy depends on the specific characteristics of each tumor, as has been observed in the first analyses of 216 patients treated by standard anthracycline-based neo-adjuvant chemotherapy in the nine-center, national I-SPY1 trial (CALGB 150007/150012, ACRIN 6657) [52-55]. In this trial patients had serial MRI and core biopsies performed at baseline, after one cycle, during treatment, and before surgery to identify markers of tumor response. Full-genome gene expression data on pre-treatment biopsies were collected, as were outcome data for initial tumor response (pathological assessment) and 3-5-year outcome data. These data are used in this study for a retrospective prevalence check of identified biomarkers for response prediction to PARP inhibition.

Following on I-SPY1, I-SPY2 is a neoadjuvant trial for women with high risk, locally advanced primary breast cancer (>3.0 cm) where response to treatment and measurement of pathologic complete response is the endpoint. The I-SPY2 trial (http://ispy2.org/) will compare the efficacy of phase 2 investigational agents—among which the PARP inhibitor ABT-888—in combination with standard chemotherapy with the efficacy of standard therapy alone in approximately 800 women with locally advanced stage II or III breast cancer [Barker A D, Sigman C C, Kelloff G J, Hylton N M, Berry D A, Esserman L J: I-SPY 2: an adaptive breast cancer trial design in the setting of neoadjuvant chemotherapy. Clinical pharmacology and therapeutics 2009, 86(1):97-100]. Due to the Bayesian nature of the trial, investigational agents can be graduated or dropped much faster based on continuous information accrual during the trial, allowing more agents to be tested more efficiently [Berry D A: Bayesian clinical trials. Nature reviews Drug discovery 2006, 5(1):27-36]. This trial has in addition been set up to test and refine cell line based predictors of response to PARP inhibitors and other investigational agents.

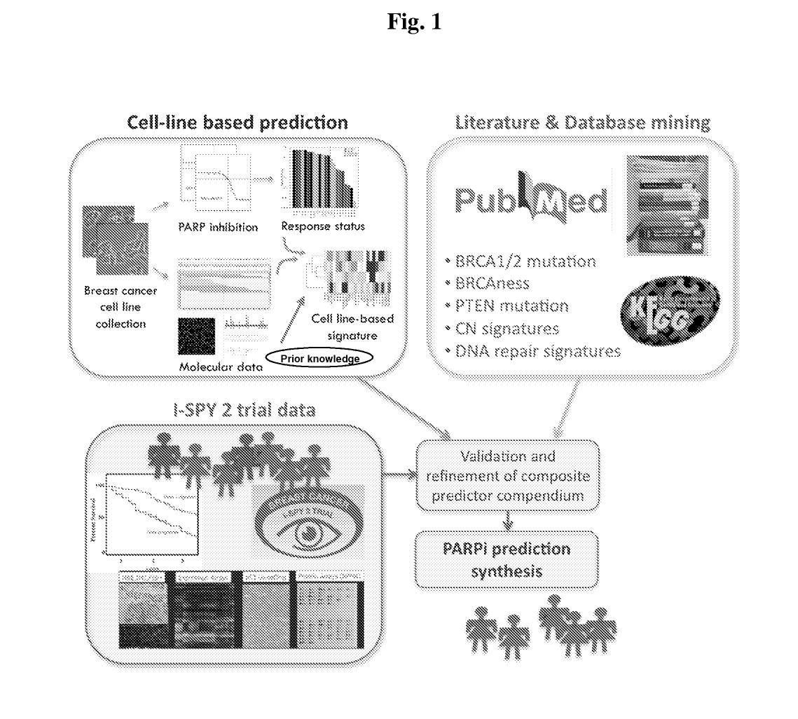

There are therapeutic agents that have been approved by FDA for specific subgroups of breast cancer patients, such as ERBB2-positive and triple-negative tumors. However, molecular signatures are needed when the responding subgroup cannot clearly be defined based on markers measurable with immunohistochemistry [Sotiriou C, Pusztai L: Gene-expression signatures in breast cancer. The New England journal of medicine 2009, 360(8):790-800]. This is the case for PARP inhibitors. There is therefore an urgent need to understand why some clinical trials succeeded and others failed. Moreover, there is the hypothesis that deficiency in other genes involved in the HR pathway besides BRCA1/2 may confer sensitivity to PARP inhibitors. As this would broaden the applicability to sporadic cancers with defects in HR-directed repair, development of biomarkers for prediction of sensitivity to PARP inhibitors is required to guide new clinical trials in patient selection in the future. We used a breast cancer cell line panel with available baseline molecular data and response to Olaparib for the validation of markers described so far in literature as well as for the development of new markers. In the near future, our findings will be validated and refined in I-SPY2 for the PARP inhibitor ABT-888. An overview of our approach is shown in FIG. 1.

Cell Line Panel with Drug Response Data.

For the validation of previously described markers and the development of new markers influenced by PARP inhibition, a panel of breast cancer cell lines was used [58, 88]. Seven data types covering the full molecular range were collected for a set of 72 breast cancer cell lines: copy number (Affymetrix SNP6), gene expression (Affymetrix U133A, Exon array), transcriptome sequencing (Illumina GAII), methylation (Illumina BeadChip), protein abundance (reverse protein lysate array), mutation status (COSMIC), and RNA interference viability screening (siRNA). All data sets were accordingly preprocessed. This cell line panel mirrors many of the molecular characteristics of the tumors from which they were derived, and are thus a good preclinical model for the study of drug response in cancer [Neve R M, Chin K, Fridlyand J, Yeh J, Baehner F L, Fevr T, Clark L, Bayani N, Coppe J P, Tong F et al: A collection of breast cancer cell lines for the study of functionally distinct cancer subtypes. Cancer cell 2006, 10(6):515-527]. Hierarchical clustering of breast cancer cell lines with primary breast cancers based on pathway activity has shown that deregulated pathways are better associated with transcriptional subtype than origin (i.e., tumor vs. cell line) [Heiser L M, et al., (2012) Subtype and pathway specific responses to anticancer compounds in breast cancer. Proceedings of the National Academy of Sciences of the United States of America 109 (8):2724-2729].

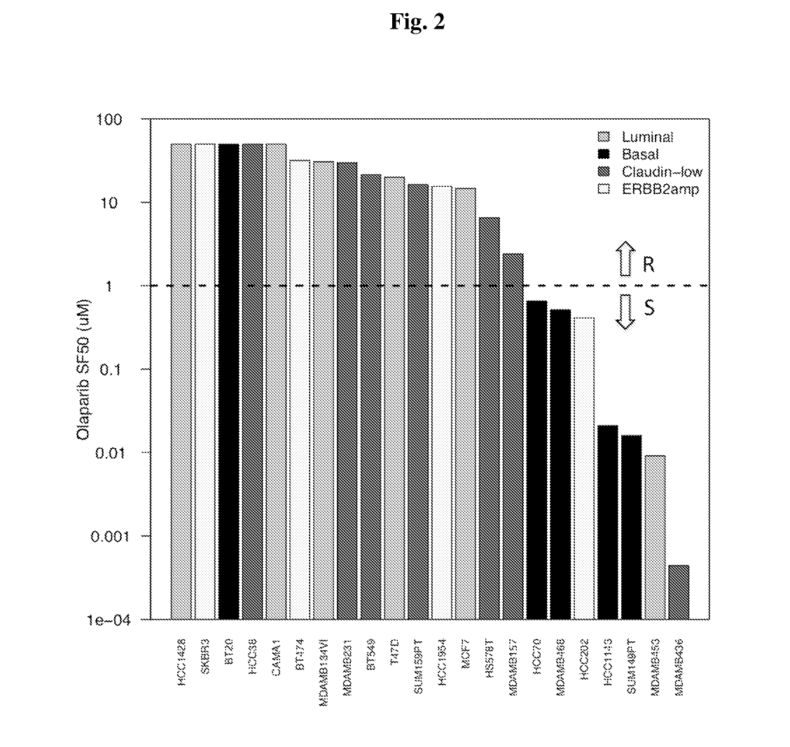

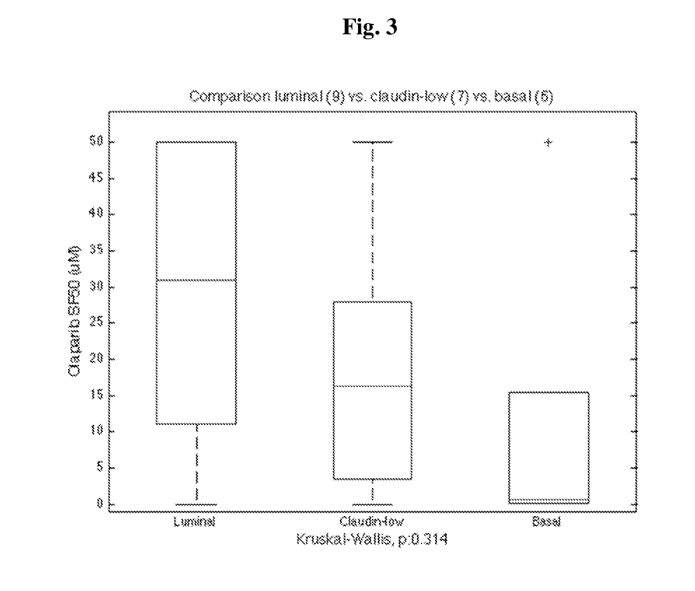

Thirty-three breast cancer cell lines were tested for response to Olaparib, of which 22 with molecular data. Survival fraction at 50% (SF50) was used as drug response measure. FIG. 2 shows the waterfall plot of SF50 for the 22 cell lines used in this study, ordered from most resistant at the left to most sensitive at the right. Among those, 6 were basal with HCC1954 in addition ERBB2 amplified, 7 claudin-low and 9 luminal of which 3 ERBB2 amplified. A trend was observed towards more sensitivity in the basal subtype and more resistance in the luminal cell lines, although not significant due to the low number of cell lines (Kruskal-Wallis test, p-value 0.314; FIG. 3). Drug response did not differ between ERBB2 amplified and non-ERBB2 amplified cell lines (Wilcoxon rank sum test, p-value 0.578). For further analyses, the cell lines were divided into a group of 13 resistant and 9 sensitive cell lines, based on an SF50 threshold of 9, corresponding to the largest change in slope for SF50 (FIG. 2). Table 2 gives an overview of the 22 cell lines and the molecular data sets available for each of them.

Validation of Literature Markers in Our Cell Line Panel.

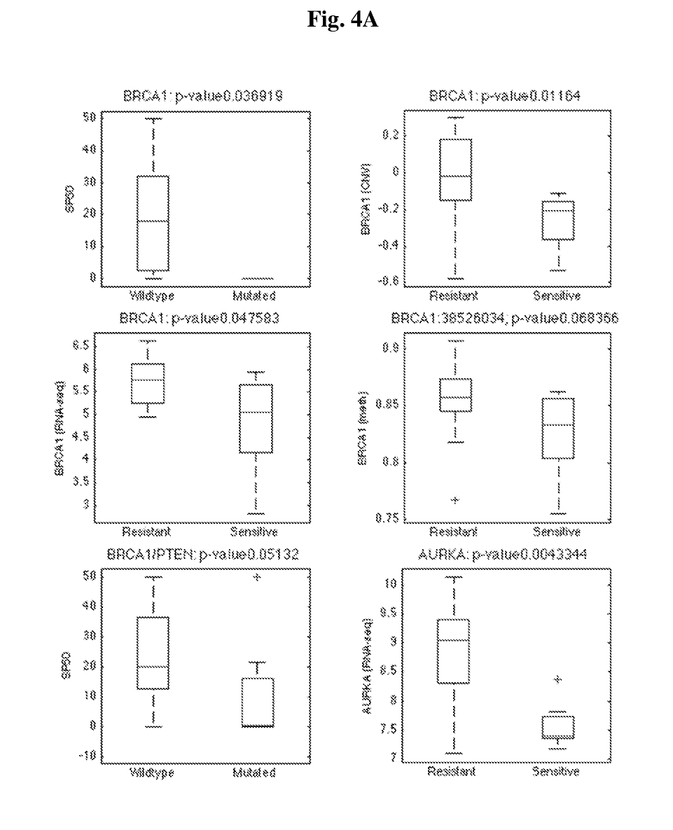

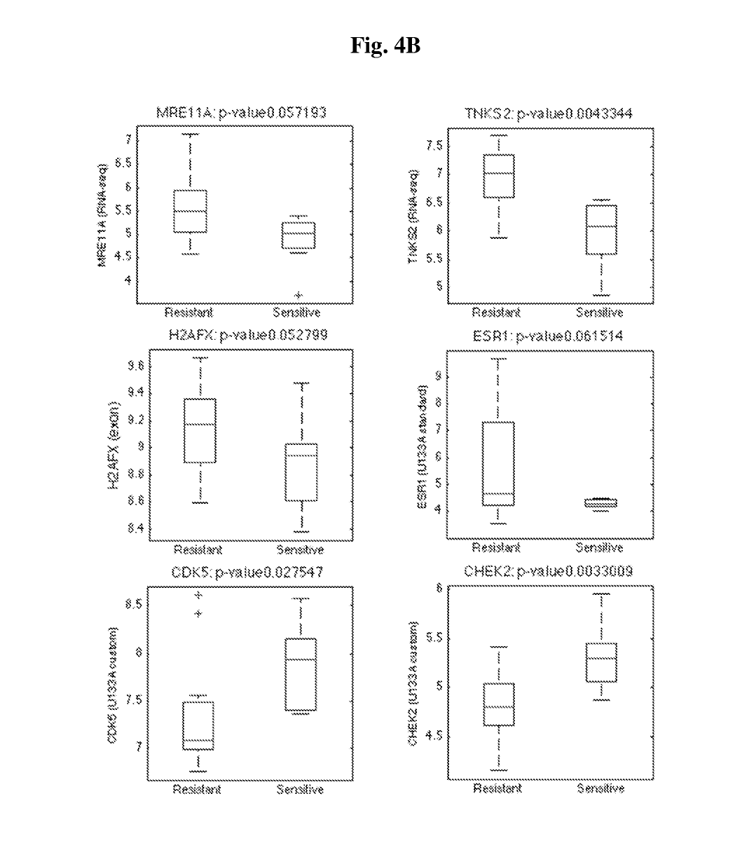

For the validation of the markers from literature in our set of breast cancer cell lines, the non-parametric Wilcoxon rank sum test was used. Table 4 shows the results per set of markers (mutations, expression, copy number, promoter methylation, siRNA). Biomarkers from literature that were found to be significant in our cell line panel are shown in FIG. 4A and FIG. 4B.

Mutation status for the 11 genes in Table 3 was obtained from COSMIC v53. Only genes with a mutation in at least 1/22 cell lines are included in Table 4a. BRCA1-mutated cell lines were more sensitive to Olaparib compared to the wildtype cell lines (p-value 0.037). Although PTEN mutation status on its own was not significantly related to Olaparib response (p-value 0.511), mutation status in BRCA1 and PTEN were combined due to the strong association in breast cancer between BRCA1 mutation and lost PTEN expression [59]. In that case, cell lines with a mutation in either of both genes were more sensitive to Olaparib than cell lines that were wildtype for both genes (p-value 0.051). For TP53, a distinction in mutation type was made as a higher incidence of protein truncating TP53 mutations were observed in BRCA1-mutated and basal-like breast cancers [28]. According to the COSMIC database, however, 12/13 mutated cell lines had a missense mutation in TP53, and MDAMB157 was characterized by a frameshift mutation. Results for the association of gene expression with Olaparib response are shown in Table 4b for the three platforms (U133A, exon array and RNA-seq). Genes APEX1, AURKA, BRCA1, EMSY, ESR1, FANCD2, 2H2AX, MRE11A, PGR, and TNKS2 were significantly down-regulated in the sensitive compared to the resistant cell lines, according to at least 1 platform. Down-regulation of ESR1 and PGR was confirmed at protein level with RPPA (p-value 0.126 and 0.059, respectively). Genes CDK5, CHEK2, HMGA1, STK22C, and XRCC3 were mainly up-regulated in the sensitive compared to the resistant lines.

Results on copy number variations are shown in Table 4c, with a significant lower copy number of BRCA1 in the sensitive with respect to resistant cell lines (p-value 0.012). For high-grade, serous ovarian cancer, it has been shown that BRCA1 is inactivated by mutually exclusive genomic and epigenomic mechanisms, with germline or somatic BRCA1/2 mutations in 20% of cases, and loss of BRCA1 expression through DNA hypermethylation in 11% of cases [60]. Association of Olaparib response with methylation of the promoter region of BRCA1 was therefore determined on the subset of BRCA1-wildtype cell lines, with exclusion of the two BRCA1-mutated cell lines MDAMB436 and SUM149PT. However as can be seen in Tables 4c and 4d, BRCA1 down-regulation in our cell lines is caused by LOH with no promoter hypermethylation. None of the siRNA markers suggested in [51] were found to be significantly associated with Olaparib response in our cell line panel (Table 4e).

Cell Line-Based Predictor of Response to Olaparib.

Besides validation of suggested markers in literature, we also used the breast cancer cell line panel to identify a set of markers that can be applied to the full spectrum of breast cancer, covered by the cell line panel (that is, basal, luminal and claudin-low). Individual markers reported in literature have their limitations. Fong and colleagues, for example, showed that not all BRCA1 or BRCA2 carriers with breast cancer in their study responded to Olaparib [22]. HR defects and sensitivity to PARP inhibition might depend on the specific mutation [61, 62], and secondary BRCA2 mutations have been observed that restore BRCA1 function and thus the HR pathway [8, 63]. For PARP inhibitors, an optimal, unifying set of markers that is not restricted to triple negative breast cancer and reflects HR deficiency is still lacking. BRCA-ness has been pragmatically defined as triple negative breast cancer (and serous ovarian cancer), although data on BRCA1 methylation, FANCF methylation and EMSY amplification has indicated that up to 25% of sporadic breast cancer patients could show BRCA-ness phenotypes [21].

Our aim was to develop a genomic signature for prediction of sensitivity to a PARP inhibitor that might work for multiple PARP inhibitors and expression platforms. To obtain robust predictive markers that are minimally dependent on the specific PARP inhibitor and expression platform, the bottom-up approach was opted for, restricted to genes related to a biological or molecular pathway or specific biological phenotypes [57]. First, prior knowledge of six principal DNA repair pathways for the maintenance of genomic integrity was incorporated, being BER, NER, MMR, DDR, HR and NHEJ (Kyoto Encyclopedia of Genes and Genomes (KEGG) database release 55.1 [Kanehisa M, Goto S, Furumichi M, Tanabe M, Hirakawa M: KEGG for representation and analysis of molecular networks involving diseases and drugs. Nucleic acids research 2010, 38(Database issue):D355-360]+ literature mining [Wang X, Weaver D: The ups and downs of DNA repair biomarkers for PARP inhibitor therapies. Am J Cancer Res 2011, 1(3):301-327], with the analysis for the latter restricted to the key biomarkers shown in bold in Table 1). All 118 genes from these pathways were included in the analysis due to crosstalk between DNA repair pathways that operate at different functional levels in cells. Secondly, stringent criteria for biomarker inclusion were applied using three different platforms for expression measurement (U133A with standard or custom annotation, exon array and RNA-seq).

For each DNA repair pathway and expression data set, logistic regression with forward selection (5-fold CV) was repeated 100 times to determine the most important markers selected in over half of the iterations and shown in Table 5. These markers were further reduced to those selected with consistent pattern of sensitivity for at least 2 out of 3 platforms. Eight genes fulfilled the criteria, of which 5 were resistance markers (BRCA1, H2AFX, MRE11A, TDG and XRCC5) and 3 sensitivity markers (BRCA2, CHEK1 and CHEK2) (see Table 5). For a resistance marker, higher expression results in a lower predicted probability of response, whilst for a sensitivity marker, higher expression is related to a higher probability of response. The heatmap of the expression of the 8 genes measured on U133A with use of standard annotation is shown in FIG. 5a for the cell line panel and the data is shown in Table 8.

Eight Biomarkers for Prediction of Response to PARP Inhibition in Breast Cancer.

In one embodiment, the signature for response prediction to Olaparib comprising eight genes, of which 5 were found to be resistance markers (BRCA1, H2AFX, MRE11A, TDG and XRCC5) and 3 were found to be sensitivity markers (BRCA2, CHEK1 and CHEK2). For a resistance marker, higher expression in a patient results in a lower predicted probability of response to a PARP inhibitor, whilst for a sensitivity marker, higher expression in a patient is related to a higher probability of response to a PARP inhibitor.

BRCA1 (breast cancer 1, early onset; gene ID 672) is involved in DSB repair via RAD51-mediated HR, DNA damage signaling and cell cycle checkpoint regulation. Mutations in BRCA1, loss of heterozygosity at the BRCA1 locus and deregulated expression have been described in literature as potential markers for prediction of response to PARP inhibitors. In our signature, down-regulation of BRCA1 is a predictor of sensitivity.

The expression level of a gene encoding BRCA1 can also be measured by using or detecting the human nucleotide sequence, or a fragment thereof, of GenBank Accession No. NM_{—}007294.3 GI:237757283, Homo sapiens breast cancer 1, early onset (BRCA1), transcript variant 1, mRNA, (SEQ ID NO: 1); GenBank Accession No. NM_{—}007300.3 GI:237681118, Homo sapiens breast cancer 1, early onset (BRCA1), transcript variant 2, mRNA, (SEQ ID NO: 2); GenBank Accession No. NM_{—}007297.3 GI:23768112, Homo sapiens breast cancer 1, early onset (BRCA1), transcript variant 3, mRNA, (SEQ ID NO: 3); GenBank Accession No. NM_{—}007298.3 GI:237681122, Homo sapiens breast cancer 1, early onset (BRCA1), transcript variant 4, mRNA, (SEQ ID NO: 4); GenBank Accession No. NM_{—}007299.3 GI:237681124, Homo sapiens breast cancer 1, early onset (BRCA1), transcript variant 5, mRNA, (SEQ ID NO: 5), the GenBank Accession and GeneID information hereby incorporated by reference.

The BRCA1 mRNAs (SEQ ID NOS:1-5) are expressed as the breast cancer type 1 susceptibility protein isoform 1 to isoform 5 [Homo sapiens](BRCA1) protein having GenBank Accession Nos. NP_{—}009225.1 GI:6552299 (SEQ ID NO: 19); NP_{—}009231.2 GI:237681119 (SEQ ID NO:20); NP_{—}009228.2 GI:237681121 (SEQ ID NO:21); NP_{—}009229.2 GI:237681123 (SEQ ID NO:22); NP_{—}009230.2 GI:237681125 (SEQ ID NO:23), the GenBank Accession and GeneID information are hereby incorporated by reference.

BRCA2 (breast cancer 2, early onset; gene ID 675) is also involved in DSB repair via RAD5′-mediated HR, it interacts with RAD51, and translocates RAD51 to the site of damaged DNA for repair initiation. Breast cancer patients who carry a BRCA2 mutation have been shown to be more sensitive to PARP inhibitors due to an HR defect. In our cell line panel, overexpression of BRCA2 is a predictor of sensitivity. According to Turner and colleagues, BRCA2-like samples are characterized by EMSY amplification. In the cell line panel, however, sensitive cell lines had a lower EMSY copy number level than resistant cell lines (p-value 0.18), suggesting that BRCA2-associated cell lines are more resistant/less sensitive.

The expression level of a gene encoding BRCA2 can also be measured by using or detecting the human nucleotide sequence, or a fragment thereof, of Homo sapiens breast cancer 2, early onset (BRCA2), mRNA (GenBank Accession No. NM_{—}000059.3 GI:119395733; SEQ ID NO: 6) sequence is provided in the Sequence Listing as SEQ ID NO: 6, and is expressed as the breast cancer type 2 susceptibility protein [Homo sapiens], GenBank Accession No: NP_{—}000050.2 GI:119395734 (SEQ ID NO:24), hereby incorporated by reference.

Compositions and methods for the detection of BRCA1 amplification and expression levels are described in the art and by U.S. Pat. Nos. 5,693,473; 5,709,999; 5,710,001; 5,753,441; 5,837,492 and 5,905,026, all of which are hereby incorporated by reference.

CHEK1 (CHK1 checkpoint homolog; gene ID 1111) and CHEK2 (CHK2 checkpoint homolog; gene ID 11200) are kinases with signal transduction function in cell cycle regulation and checkpoint responses. They are involved in the two major parallel DDR pathways, ATR-Chk1 and ATM-Chk2. Tumor cells with deficiency of DDR have been suggested to be hypersensitive to PARP inhibitors, with the DNA repair biomarker CHEK1 shown to be overexpressed in BRCA1-like versus non-BRCA1-like triple negative breast cancer. In the cell line panel, both CHEK1 and CHEK2 are sensitivity markers, with overexpression related to sensitivity.

The expression level of a gene encoding CHEK1 can also be measured by using or detecting the human nucleotide sequence, or a fragment thereof, of Homo sapiens Checkpoint Kinase 1 (CHEK1), mRNA, GenBank Accession No. NM_{—}001114122.2 GI:349501056 (SEQ ID NO:7), and is expressed as serine/threonine-protein kinase Chk1 isoform 1 [Homo sapiens] NP_{—}001107594.1 GI:166295196 (SEQ ID NO:25), hereby incorporated by reference.

The expression level of a gene encoding CHEK1 can also be measured by using or detecting the human nucleotide sequence, or a fragment thereof, of Homo sapiens Checkpoint Kinase 1 (CHEK1), transcript variant 4, mRNA, GenBank Accession No. NM_{—}001244846.1 GI:349501060 (SEQ ID NO:8); which is expressed as serine/threonine-protein kinase Chk1 isoform 2 [Homo sapiens] GenBank Accession No. NP_{—}001231775.1 GI:349501061 (SEQ ID NO:26), hereby incorporated by reference.

The expression level of a gene encoding CHEK2 can also be measured by using or detecting the human nucleotide sequence, or a fragment thereof, of Homo sapiens Checkpoint Kinase 2 (CHEK2), transcript variant 3, mRNA, GenBank Accession No. NM_{—}001005735.1 GI:54112406 (SEQ ID NO: 9); transcript variant 1, mRNA, GenBank Accession No. NM_{—}007194.3 GI:54112404 (SEQ ID NO:10); transcript variant 2, mRNA GenBank Accession No. NM_{—}145862.2 GI:54112405 (SEQ ID NO:11), which are expressed as Homo sapiens checkpoint kinase 2 (CHEK2), serine/threonine-protein kinase Chk2 isoform c [Homo sapiens] GenBank Accession No. NP_{—}001005735.1 GI:54112407 (SEQ ID NO: 27); serine/threonine-protein kinase Chk2 isoform a [Homo sapiens] GenBank Accession No. NP_{—}009125.1 GI:6005850 (SEQ ID NO:28); serine/threonine-protein kinase Chk2 isoform b [Homo sapiens] GenBank Accession No. NP_{—}665861.1 GI:22209009 (SEQ ID NO:29), all of which are hereby incorporated by reference.

MRE11A (MRE11 meiotic recombination 11 homolog A; gene ID 4361) is part of the MRN complex, a multifaceted molecular machine composed of MRE11A, RAD50 and NBS1 for DSB recognition. MRE11A interacts with RAD50 to associate with the DNA ends of a DSB, it interacts with NBS1, and has both endo- and exonuclease activities important for the initial steps of DNA end resection. PARP1 is required for rapid accumulation of MRE11A at DSB sites. Due to this direct interaction between PARP1 and MRE11A, deficiency in MRE11A may sensitize cells to PARP1 inhibition based on the concept of synthetic lethality. Moreover, a dominant negative mutation in MRE11A in mismatch repair deficient cancers has been shown to sensitize cells to agents causing replication fork stress. The MRE11A pattern in our cell line panel is consistent with literature, with down-regulation a predictor of sensitivity.

The expression level of a gene encoding MRE11A can also be measured by using or detecting the human nucleotide sequence, or a fragment thereof, of Homo sapiens MRE11 meiotic recombination 11 homolog A (S. cerevisiae) (MRE11A), transcript variant 1 GenBank Accession NO: NM_{—}005591.3 GI:56550105 (SEQ ID NO:13), and transcript variant 2, mRNA, NM_{—}005590.3 GI:56550106 (SEQ ID NO:12), which are expressed as double-strand break repair protein MRE11A isoform 2 GenBank Accession No. NP_{—}005581.2 GI:24234690 (SEQ ID NO:30) and isoform 1 NP_{—}005582.1 GI:5031923 (SEQ ID NO:31), the GenBank Accession Numbers and Gene information which is hereby incorporated by reference.

H2AFX (H2A histone family, member X; gene ID 3014) is part of the DDR pathway. γH2AX foci are formed with almost every DNA DSB in response to DNA damage or after exposure to exogenous DNA damage agents that induce DSBs. These foci are known to be involved in DSB repair by the HR and NHEJ pathways and have been suggested as marker for the evaluation of the efficacy of various DSB-inducing compounds and radiation. In the cell line panel, γH2AX acts as a resistance marker, with down-regulation pointing towards sensitivity.

The expression level of a gene encoding H2AFX can also be measured by using or detecting the human nucleotide sequence, or a fragment thereof, of Homo sapiens H2A histone family, member X (H2AFX), mRNA, GenBank Accession No. NM_{—}002105.2 GI:52630339 (SEQ ID NO:14), which is expressed as histone H2A.x [Homo sapiens] protein GenBank Accession No. NP_{—}002096.1 GI:4504253 (SEQ ID NO:32), the GenBank Accession Numbers and Gene information which is hereby incorporated by reference.

TDG (thymine-DNA glycosylase; gene ID 6996) is part of the BER pathway, and has been identified as a resistance marker.

The expression level of a gene encoding TDG can also be measured by using or detecting the human nucleotide sequence, or a fragment thereof, of Homo sapiens thymine-DNA glycosylase (TDG), mRNA, GenBank Accession No. NM_{—}003211.4 GI:197927092 (SEQ ID NO:15), which is expressed as G/T mismatch-specific thymine DNA glycosylase [Homo sapiens] protein GenBank Accession No. NP_{—}003202.3 GI:59853162 (SEQ ID NO:33), the GenBank Accession Numbers and Gene information which is hereby incorporated by reference.

XRCC5 (X-ray repair complementing defective repair in Chinese hamster cells 5 (double-strand-break rejoining); gene ID 7520) is involved in the NHEJ pathway. XRCC5 (also known as Ku80) and XRCC6 (Ku70) form the Ku heterodimer Ku70/Ku80 that localizes to DSBs within seconds to initiate NHEJ. Ku80 deficient cells have been shown to become sensitive to ionizing radiation by PARP inhibition. Also in our cell line panel, XRCC5 showed up as a resistance marker, with down-regulation pointing towards sensitivity.

The expression level of a gene encoding H2AFX can also be measured by using or detecting the human nucleotide sequence, or a fragment thereof, of Homo sapiens X-ray repair complementing defective repair in Chinese hamster cells 5 (double-strand-break rejoining) (XRCC5), mRNA, GenBank Accession No. NM_{—}021141.3 GI:195963391 (SEQ ID NO:16) which is expressed as X-ray repair cross-complementing protein 5 [Homo sapiens] protein GenBank Accession No. NP_{—}066964.1 GI:10863945 (SEQ ID NO:34), the GenBank Accession Numbers and Gene information which is hereby incorporated by reference.

Biomarker Description.

BRCA1 is involved in DSB repair via RAD5′-mediated HR, DNA damage signaling and cell cycle checkpoint regulation [Gudmundsdottir K, Ashworth A: The roles of BRCA1 and BRCA2 and associated proteins in the maintenance of genomic stability. Oncogene 2006, 25(43):5864-5874, Tutt A, Ashworth A: The relationship between the roles of BRCA genes in DNA repair and cancer predisposition. Trends in molecular medicine 2002, 8(12):571-576]. Mutations in BRCA1, loss of heterozygosity at the BRCA1 locus and deregulated expression have been described in literature as potential markers for prediction of response to PARP inhibitors [Turner N, Tutt A, Ashworth A: Hallmarks of ‘BRCAness’ in sporadic cancers. Nature reviews Cancer 2004, 4(10):814-819]. In our signature, down-regulation of BRCA1 is a predictor of sensitivity. BRCA2 is also involved in DSB repair via RAD5′-mediated HR, it interacts with RAD51, and translocates RAD51 to the site of damaged DNA for repair initiation [Gudmundsdottir K, Ashworth A: The roles of BRCA1 and BRCA2 and associated proteins in the maintenance of genomic stability. Oncogene 2006, 25(43):5864-5874, Tutt A, Ashworth A: The relationship between the roles of BRCA genes in DNA repair and cancer predisposition. Trends in molecular medicine 2002, 8(12):571-576]. Breast cancer patients who carry a BRCA2 mutation have been shown to be more sensitive to PARP inhibitors due to an HR defect [Edwards S L, Brough R, Lord C J, Natrajan R, Vatcheva R, Levine D A, Boyd J, Reis-Filho J S, Ashworth A: Resistance to therapy caused by intragenic deletion in BRCA2. Nature 2008, 451(7182):1111-1115]. In our panel, however, none of the cell lines have a mutation in BRCA2, confirmed with exome sequencing. In BRCA2-wildtype cell lines, overexpression of BRCA2 was found to be a predictor of sensitivity. CHEK1 and CHEK2 are kinases with signal transduction function in cell cycle regulation and checkpoint responses [Sancar A, Lindsey-Boltz L A, Unsal-Kacmaz K, Linn S: Molecular mechanisms of mammalian DNA repair and the DNA damage checkpoints. Annual review of biochemistry 2004, 73:39-85]. They are involved in the two major parallel DDR pathways, ATR-CHEK1 and ATM-CHEK2 [Wang X, Weaver D: The ups and downs of DNA repair biomarkers for PARP inhibitor therapies. Am J Cancer Res 2011, 1(3):301-327]. Tumor cells with deficiency of DDR have been suggested to be hypersensitive to PARP inhibitors, with the DNA repair biomarker CHEK1 shown to be overexpressed in BRCA1-like versus non-BRCA1-like triple negative breast cancer [Rodriguez A A, Makris A, Wu M F, Rimawi M, Froehlich A, Dave B, Hilsenbeck S G, Chamness G C, Lewis M T, Dobrolecki L E et al: DNA repair signature is associated with anthracycline response in triple negative breast cancer patients. Breast cancer research and treatment 2010, 123(1):189-196]. In the cell line panel, both CHEK1 and CHEK2 are sensitivity markers, with overexpression related to sensitivity. MRE11A is part of the MRN complex, a multifaceted molecular machine composed of MRE11A, RAD50 and NBS1 for DSB recognition [Williams G J, Lees-Miller S P, Tainer J A: Mre11-Rad50-Nbs1 conformations and the control of sensing, signaling, and effector responses at DNA double-strand breaks. DNA repair 2010, 9(12):1299-1306]. MRE11A interacts with RAD50 to associate with the DNA ends of a DSB, it interacts with NBS1, and has both endo- and exonuclease activities important for the initial steps of DNA end resection [Ciccia A, Elledge S J: The DNA damage response: making it safe to play with knives. Molecular cell 2010, 40(2):179-204]. PARP1 is required for rapid accumulation of MRE11A at DSB sites. Due to this direct interaction between PARP1 and MRE11A, deficiency in MRE11A may sensitize cells to PARP1 inhibition based on the concept of synthetic lethality [Vilar E, Bartnik C M, Stenzel S L, Raskin L, Ahn J, Moreno V, Mukherjee B, Iniesta M D, Morgan M A, Rennert G et al: MRE11 deficiency increases sensitivity to poly(ADP-ribose) polymerase inhibition in microsatellite unstable colorectal cancers. Cancer research 2011, 71(7):2632-2642]. Moreover, a dominant negative mutation in MRE11A in mismatch repair deficient cancers has been shown to sensitize cells to agents causing replication fork stress [Wen Q, Scorah J, Phear G, Rodgers G, Rodgers S, Meuth M: A mutant allele of MRE11 found in mismatch repair-deficient tumor cells suppresses the cellular response to DNA replication fork stress in a dominant negative manner. Molecular biology of the cell 2008, 19(4):1693-1705]. The MRE11A pattern in our cell line panel is consistent with literature, with down-regulation a predictor of sensitivity. H2AFX is part of the DDR pathway. γH2AX foci are formed with almost every DNA DSB in response to DNA damage or after exposure to exogenous DNA damage agents that induce DSBs [Banuelos C A, Banath J P, Kim J Y, Aquino-Parsons C, Olive P L: gammaH2AX expression in tumors exposed to cisplatin and fractionated irradiation. Clinical cancer research: an official journal of the American Association for Cancer Research 2009, 15(10):3344-3353, Bonner W M, Redon C E, Dickey J S, Nakamura A J, Sedelnikova O A, Solier S, Pommier Y: GammaH2AX and cancer. Nature reviews Cancer 2008, 8(12):957-967]. These foci are known to be involved in DSB repair by the HR and NHEJ pathways and have been suggested as marker for the evaluation of the efficacy of various DSB-inducing compounds and radiation [Wang X, Weaver D: The ups and downs of DNA repair biomarkers for PARP inhibitor therapies. Am J Cancer Res 2011, 1(3):301-327]. In the cell line panel, γH2AX acts as a resistance marker, with down-regulation pointing towards sensitivity. TDG is part of the BER pathway, and has been identified as a resistance marker. Finally, XRCC5 (also known as Ku80) is involved in the NHEJ pathway. XRCC5 and XRCC6 (Ku70) form the Ku heterodimer Ku70/Ku80 that localizes to DSBs within seconds to initiate NHEJ [Mahaney B L, Meek K, Lees-Miller S P: Repair of ionizing radiation-induced DNA double-strand breaks by non-homologous end-joining. The Biochemical journal 2009, 417(3):639-650]. Ku80 deficient cells have been shown to become sensitive to ionizing radiation by PARP inhibition [Wang X, Weaver D: The ups and downs of DNA repair biomarkers for PARP inhibitor therapies. Am J Cancer Res 2011, 1(3):301-327, Loser D A, Shibata A, Shibata A K, Woodbine L J, Jeggo P A, Chalmers A J: Sensitization to radiation and alkylating agents by inhibitors of poly(ADP-ribose) polymerase is enhanced in cells deficient in DNA double-strand break repair. Molecular cancer therapeutics 2010, 9(6):1775-1787]. Also in our cell line panel, XRCC5 showed up as a resistance marker, with down-regulation pointing towards sensitivity.

Signature Prevalence Validation in Tumor Samples.

The weighted voting algorithm [Moulder S, Yan K, Huang F, Hess K R, Liedtke C, Lin F, Hatzis C, Hortobagyi G N, Symmans W F, Pusztai L: Development of candidate genomic markers to select breast cancer patients for dasatinib therapy. Molecular cancer therapeutics 2010, 9(5):1120-1127] was used to build the final 8-gene predictor shown in Table 1 and based on U133A expression (standard annotation) for which 7 predictor genes fulfilled the criteria compared to 5 out of 8 genes for the two other platforms. However, the consistency in predicted probability of response to Olaparib was high between the weighted voting predictor built on U133A expression data with standard annotation and those predictors built on the other cell line expression data sets (U133A with custom annotation, exon array and RNA-seq) for all validation data sets described below with correlation coefficients ranging from 0.82 to 0.99.

Due to lack of molecular data for tumor samples treated with any of the PARP inhibitors, we used eight U133A and two U133 plus 2 data sets on primary tumors with or without metastasis, heterogeneous in both treatment and ER/PR/LN status, and with number of tumor samples varying from 61 to 289 to verify the prevalence of the 8-gene set in tumor samples and to characterize the subpopulation of patients likely to respond according to the predictor (GSE2034, GSE20271, GSE23988, GSE4922, GSE1456, GSE7390, GSE11121, GSE12093, GSE23177, GSE5460). Testing the 8-gene signature in these tumor data sets revealed that 40-48% of patients were predicted to be responsive to Olaparib (Table 6). Validation in 117 tumor samples from the I-SPY1 clinical trial revealed that 41% of 1-SPY1 patients are likely to respond to Olaparib. To verify cross-platform generalizability, the signature was additionally tested in 430 breast invasive carcinoma samples collected by TCGA (The Cancer Genome Atlas) for which custom Agilent 244K expression was available [The Cancer Genome Atlas Data Portal, available at http://tcga-data.nci.nih.gov/tcga/tcgaHome2.jsp]. Prevalence was confirmed on this distinct platform (Table 6). Because genes that are consistently up-regulated in a set of cell lines should also be concurrently up-regulated in tumor samples, and similar for genes that are consistently down-regulated, we calculated the Jaccard similarity coefficient [Van Rijsbergen C: Information retrieval: Butterworth; 1979]. This coefficient ranges from 0 to 1 and reflects the similarity in co-expression pattern between cell lines and tumor samples. In our panel, the Jaccard coefficient was on average 0.551 with standard deviation 0.101 (min-max=[0.429 0.75]) (Table 6).

Finally, to characterize the patient population likely to respond to a PARP inhibitor, breast cancer subtype was associated with response prediction to Olaparib in the I-SPY1 and TCGA tumor sets (Table 7). For both data sets, normal-like tumor samples were excluded from the analysis, resulting in 113 I-SPY1 and 422 TCGA samples. A trend was observed towards a higher percentage of basal and luminal A samples and a lower percentage of luminal B and ERBB2-amplified samples in the set of samples predicted to respond to Olaparib (p-values 0.109 and 0.014 for I-SPY1 and TCGA, respectively; Table 7).

Thus, in one embodiment, herein are provided the measurement and detection of gene amplification levels and expression levels of a gene as measured from a sample from a patient that comprises essentially a cancer cell or cancer tissue of a cancer tumor. Such methods for obtaining such samples are well known to those skilled in the art. When the cancer is breast cancer, the amplification and expression levels of a gene are measured from a sample from the patient that comprises essentially a breast cancer cell or breast cancer tissue of a breast cancer tumor.

As used herein, the term “gene amplification” is used in a broad sense, referring to an increase, decrease or change in gene copy number, and can also comprise assessment of amplification levels of the gene's expression and gene product. Thus, levels of gene expression, as well as corresponding protein expression can be evaluated. In the embodiments that follow, it is understood that assessment of gene expression can be used to assess level of gene product such as RNA or protein.

Methods for detection of expression levels of a gene can be carried out using known methods in the art including but not limited to, fluorescent in situ hybridization (FISH), immunohistochemical analysis, comparative genomic hybridization, PCR methods including real-time and quantitative PCR, in situ hybridization for RNA, immunohistochemistry and reverse phase protein lysate arrays for protein and other sequencing and analysis methods. The expression level of the gene in question can be measured by measuring the amount or number of molecules of mRNA or transcript in a cell. The measuring can comprise directly measuring the mRNA or transcript obtained from a cell, or measuring the cDNA obtained from an mRNA preparation thereof. Such methods of extracting the mRNA or transcript from a cell, or preparing the cDNA thereof are well known to those skilled in the art. In other embodiments, the expression level of a gene can be measured by measuring or detecting the amount of protein or polypeptide expressed, such as measuring the amount of antibody that specifically binds to the protein in a dot blot or Western blot. The proteins described in the present invention can be overexpressed and purified or isolated to homogeneity and antibodies raised that specifically bind to each protein. Such methods are well known to those skilled in the art.

Comparison of the detected expression level of a gene in a patient sample is often compared to the expression levels detected in a normal tissue sample or a reference expression level. In some embodiments, the reference expression level can be the average or normalized expression level of the gene in a panel of normal cell lines or cancer cell lines. In some embodiments, the detected gene copy number levels in a patient sample are compared to gene copy number levels in a normal tissue sample or reference gene copy number level.

Thus, embodiments of the invention include: A method for predicting the response of a patient with breast cancer, said method comprising: providing breast cancer tissue from the patient; determining from the provided tissue, the level of gene amplification or gene expression for at least one of the following genes: BRCA1, BRCA2, H2AFX, MRE11A, TDG, XRCC5, CHEK1 or CHEK2; identifying that the at least one gene or gene product is amplified; whereby, when the at least one gene or gene product is amplified, this is an indication that the patient is predicted to be sensitive or resistant to a PARP inhibitor. This method can comprise that the amplification and/or expression levels of the gene or gene product are detected.

In one embodiment, the expression level of a gene encoding protein can be measured using a nucleotide fragment, an oligonucleotide derived from or a probe that hybridizes to the nucleotide sequence(s) or a fragment thereof of at least one of the genes BRCA1, BRCA2, H2AFX, MRE11A, TDG, XRCC5, CHEK1 or CHEK2 (SEQ ID NOS:1-16). In another embodiment, a protein selected from one of SEQ ID NOs: 19-34 can be detected and protein levels measured using techniques as known in the art and described herein. In another embodiment, the expression products of at least one of the genes BRCA1, BRCA2, H2AFX, MRE11A, TDG, XRCC5, CHEK1 or CHEK2 are measured using techniques as known in the art.

An increase in the amplification or expression level of one or more of the 5 resistance markers (BRCA1, H2AFX, MRE11A, TDG or XRCC5) in the patient sample, as compared to the amplification or expression level of each gene in a normal tissue sample or a reference expression level (such as the average expression level of the gene in a cell line panel or a cancer cell or tumor panel, or the like), indicates that the cancer cell, tissue or tumor, from which the patient sample was obtained, is resistant to treatment with a PARP inhibitor. In some embodiments, an increase in the amplification or expression levels of any one or more of the 3 sensitivity markers (BRCA2, CHEK1 or CHEK2) in the patient sample, as compared to the amplification or expression level of each gene in a normal tissue sample or a reference expression level (such as the average expression level of the gene in a cell line panel or a cancer cell or tumor panel, or the like), indicates that the cancer cell, tissue or tumor, from which the patient sample was obtained, is sensitive to treatment with a PARP inhibitor.

In another embodiment, a decrease in the amplification or expression level of a gene in the patient sample, as compared to the amplification or expression level of a gene in a normal tissue sample, and a modulation in the expression level of one or more of the following genes, BRCA1, H2AFX, MRE11A, TDG or XRCC5, in the patient sample, as compared to the amplification or expression level of each gene in the normal tissue sample, indicates that the cancer cell, tissue or tumor, from which the patient sample was obtained, is sensitive to treatment with a PARP inhibitor. In some embodiments, decrease in the amplification or expression levels of any one, or more of BRCA2, CHEK1 or CHEK2 in the patient sample, as compared to the expression level of each gene in a normal tissue sample, indicates that the cancer cell, tissue or tumor, from which the patient sample was obtained, is resistant to treatment with a PARP kinase inhibitor.

Thus, a method for identifying a cancer patient suitable for treatment with a PARP inhibitor compound, comprising: (a) measuring amplification or expression levels of a gene selected from the group consisting of genes encoding BRCA1, BRCA2, H2AFX, MRE11A, TDG, XRCC5, CHEK1 and CHEK2 in a sample from the patient; and (b) comparing the amplification or expression level of the gene from the patient with amplification or expression level of the gene in a normal tissue sample or a reference expression level, wherein an increase of amplification or expression of the gene encoding BRCA2, CHEK1 or CHEK2 and/or a decrease of amplification or expression of the gene encoding BRCA1, H2AFX, MRE11A, TDG or XRCC5 indicates the patient will be suitable for treatment with the PARP inhibitor.

In some embodiments, the method for identifying a cancer patient suitable for treatment with a PARP inhibitor compound, comprising: (a) measuring amplification or expression levels of a gene selected from the group consisting of genes encoding H2AFX, MRE11A, TDG, XRCC5, CHEK1 and CHEK2 in a sample from the patient; and (b) comparing the amplification or expression level of the gene from the patient with amplification or expression level of the gene in a normal tissue sample or a reference expression level, wherein an increase of amplification or expression of the gene encoding CHEK1 or CHEK2 and/or a decrease of amplification or expression of the gene encoding H2AFX, MRE11A, TDG or XRCC5 indicates the patient will be suitable for treatment with the PARP inhibitor. In some embodiments, step (a) measuring amplification or expression levels of at least two, three, four, five or more genes selected from the group consisting of genes encoding H2AFX, MRE11A, TDG, XRCC5, CHEK1 and CHEK2 in a sample from the patient. In another embodiment, measuring amplification or expression levels of at least one gene from the resistant group (H2AFX, MRE11A, TDG or XRCC5) and one from the sensitive group (CHEK1 or CHEK2).

In some embodiments, the method for identifying a cancer patient suitable for treatment with a PARP inhibitor compound, comprising: (a) measuring amplification or expression levels of a gene selected from the group consisting of genes encoding H2AFX, MRE11A, TDG, and XRCC5, in a sample from the patient; and (b) comparing the amplification or expression level of the gene from the patient with amplification or expression level of the gene in a normal tissue sample or a reference expression level, wherein an increase of amplification or expression of the gene encoding H2AFX, MRE11A, TDG or XRCC5 indicates the patient will be resistant to treatment with a PARP inhibitor and a decrease of amplification or expression of the gene encoding H2AFX, MRE11A, TDG or XRCC5 indicates the patient will be suitable for treatment with the PARP inhibitor.

Seven Biomarkers for Prediction of Response to PARP Inhibition in Breast Cancer.

In one embodiment, the signature for response prediction to Olaparib comprising seven genes, of which 5 were found to be resistance markers (BRCA1, MRE11A, NBS1, TDG and XPA) and 2 were found to be sensitivity markers (CHEK2 and MK2). For a resistance marker, higher expression in a patient results in a lower predicted probability of response to a PARP inhibitor, whilst for a sensitivity marker, higher expression in a patient is related to a higher probability of response to a PARP inhibitor. In some embodiments, the method for identifying a cancer patient suitable for treatment with a PARP inhibitor compound, comprising: (a) measuring amplification or expression levels of a gene selected from the group consisting of genes encoding BRCA1, MRE11A, TDG, CHEK2, MK2, NBS1 and XPA in a sample from the patient; and (b) comparing the amplification or expression level of the gene from the patient with amplification or expression level of the gene in a normal tissue sample or a reference expression level, wherein an increase of amplification or expression of the gene encoding MK2 or CHEK2 and/or a decrease of amplification or expression of the gene encoding MRE11A, TDG, BRCA1, NBS1 or XPA indicates the patient will be suitable for treatment with the PARP inhibitor.

See the above description of the genes BRCA1, MRE11A, TDG, and CHEK2 as these four genes in the present set of seven biomarkers overlap or are the same as four genes in the set of eight biomarkers described above.

MK2 (Homo sapiens mitogen-activated protein kinase-activated protein kinase 2 (MAPKAPK2; Gene ID 9261) is a member of the Ser/Thr protein kinase family. MK2 is a component of the p38 signaling pathway and is activated directly downstream of p38. This kinase is regulated through direct phosphorylation by p38 MAP kinase. The p38/MK2 signaling complex is considered to be a general stress response pathway, which is activated in response to a variety of stimuli including various toxins, osmotic stress, heat shock, reactive oxygen species, cytokines and DNA damage. MK2 activity is critical for prolonged checkpoint maintenance through a process of posttranscriptional mRNA stabilization and is a downstream effector kinase in the DNA damage response. Silencing of MK2 has been shown to exhibit synthetic lethality in the context of p53 deficiency in the presence of DNA damage suggesting suitability as a potential marker for prediction of sensitivity to PARP inhibition.

The expression level of a gene encoding MK2 can also be measured by using or detecting the human nucleotide sequence, or a fragment thereof, of GenBank Accession No. NM_{—}004759.4 GI:341865587, Homo sapiens mitogen-activated protein kinase-activated protein kinase 2 (MAPKAPK2), transcript variant 1, mRNA (SEQ ID NO: 35); GenBank Accession No. NM_{—}032960.3 GI:341865588, Homo sapiens mitogen-activated protein kinase-activated protein kinase 2 (MAPKAPK2), transcript variant 2, mRNA (SEQ ID NO:36), the GenBank Accession and GeneID information hereby incorporated by reference. The MK2 mRNAs (SEQ ID NOS:35-36) are expressed as MAP kinase-activated protein kinase 2 isoform 1 [Homo sapiens] protein having GenBank Accession No. NP_{—}004750.1 GI:1086390 (SEQ ID NO:37) and MAP kinase-activated protein kinase 2 isoform 2 [Homo sapiens] having GenBank Accession No. NP_{—}116584.2 GI:32481209 (SEQ ID NO:38), the GenBank Accession and GeneID information are hereby incorporated by reference.

NBS1 (Nijmegen breakage syndrome 1 (nibrin); gene ID 4683) is involved in DNA double-strand break repair and DNA damage-induced checkpoint activation as a member of the MRE11/RAD50 double-strand break repair multimeric complex which rejoins double-strand breaks predominantly by homologous recombination repair and collaborates with cell-cycle checkpoints at S and G2 phase to facilitate DNA repair. NBS1 is also associated with telomere maintenance and DNA replication. NBS1-deficient cells display reductions in both gene conversion and sister-chromatid exchanges (SCEs) and have been described in literature as a potential marker for prediction of sensitivity to PARP inhibition.

The expression level of a gene encoding NBS1 can also be measured by using or detecting the human nucleotide sequence, or a fragment thereof, of GenBank Accession No. NM_{—}002485.4 GI:67189763, Homo sapiens nibrin (NBN), mRNA (SEQ ID NO: 39), which is expressed as nibrin [Homo sapiens] protein, GenBank Accession No. NP_{—}002476.2 GI:33356172 (SEQ ID NO:40), the GenBank Accession Numbers and Gene information which is hereby incorporated by reference.

XPA (Homo sapiens xeroderma pigmentosum, complementation group A (XPA); gene ID 7507) is a gene that encodes a zinc finger protein involved in DNA excision repair. The encoded protein is part of the NER (nucleotide excision repair) complex which is responsible for repair of UV radiation-induced photoproducts and DNA adducts induced by chemical carcinogens. PARP inhibitor have been shown to enhance lethality in XPA deficient cells after UV irradiation.

The expression level of a gene encoding XPA can also be measured by using or detecting the human nucleotide sequence, or a fragment thereof, of GenBank Accession No. NM_{—}000380.3 GI:156564394, Homo sapiens xeroderma pigmentosum, complementation group A (XPA), transcript variant 1, mRNA (SEQ ID NO: 41), which is expressed as DNA repair protein complementing XP-A cells [Homo sapiens] protein GenBank Accession No. NP_{—}000371.1 GI:4507937 (SEQ ID NO:42) or GenBank Accession No. NR_{—}027302.1 GI:224809400, Homo sapiens xeroderma pigmentosum, complementation group A (XPA), transcript variant 2, non-coding RNA (SEQ ID NO:43), the GenBank Accession Numbers and Gene information which is hereby incorporated by reference.

It is contemplated that in some embodiments, a method for identifying a cancer patient suitable for treatment with a PARP inhibitor, comprising: (a) measuring the amplification or expression level of the group of genes encoding BRCA1, MRE11A, TDG and CHEK2; (b) measuring the amplification or expression level of at least one gene selected from the group consisting of the genes encoding H2AFX, XRCC5, BRCA2, CHEK1, MK2, NBS1 and XPA in a sample from the patient; and (b) comparing the amplification or expression level of said genes from the patient with the amplification or expression level of the genes in a normal tissue sample or a reference amplification or expression level. Thus, in some embodiments, in step (b) measuring amplification or expression levels of at least two, three or more genes selected from the group consisting of genes encoding H2AFX, XRCC5, BRCA2, CHEK1, MK2, NBS1 and XPA in a sample from the patient. In other embodiments, the group further comprising the genes encoding H2AFX, XRCC5, BRCA2, and CHEK1, in a MK2, NBS1 and XPA in a sample from the patient.

In some embodiments of the invention, the nucleotide sequence of a suitable fragment of the gene is used, or an oligonucleotide derived thereof. The length of the oligonucleotide of any suitable length. A suitable length can be at least 10 nucleotides, 20 nucleotides, 50 nucleotides, 100 nucleotides, 200 nucleotides, or 400 nucleotides, and up to 500 nucleotides or 700 nucleotides. A suitable nucleotide is one which binds specifically to a nucleic acid encoding the target gene and not to the nucleic acid encoding another gene.

In some embodiments, the method comprises measuring the expression level of ERBB2 of the patients in order to determine whether the patient is an ERBB2-negative patient. The expression level of a gene encoding ERBB2 can be measured using an oligonucleotide derived from the mouse v-erb-b2 erythroblastic leukemia viral oncogene homolog 2, neuro/glioblastoma derived oncogene homolog (avian) (Erbb2), mRNA sequence of GenBank Accession No. NM_{—}001003817.1 GI:54873609, hereby incorporated by reference and shown as SEQ ID NO: 17.

The expression level of a gene encoding ERBB2 can also be measured using or detecting the nucleotide sequence or a fragment thereof derived from the human nucleotide sequence of GenBank Accession No. NM_{—}004448.2 GI:54792095, Homo sapiens v-erb-b2 erythroblastic leukemia viral oncogene homolog 2, neuro/glioblastoma derived oncogene homolog (avian) (ERBB2), transcript variant 1, mRNA, hereby incorporated by reference and shown as SEQ ID NO: 18.

Methods of assaying for ERBB2 or HER2 protein overexpression include methods that utilize immunohistochemistry (IHC) and methods that utilize fluorescence in situ hybridization (FISH). A commercially available IHC test is DAKO HercepTest® (DAKO Corp., Carpinteria, Calif.). Patient samples having an IHC staining score of 0-1.2 is normal, and scores of 2+ may be borerderline, while results of 2.3+ are scored as positive for multiple copies of HER2 (HER2 positive).

A commercially available FISH test is PathVysion® (Vysis Inc., Downers Grove, Ill.). The HER2 genomic copy number of a patient sample is determined using FISH. Generally if a sample is found to have 3.6 or more copies of HER2 (normal=2 copies), the patient is determined to be HER2 positive.

While many HER2-positive patients suffer from metastatic breast cancer, a patient's HER2 status can also be determined in relation to other types of cancers including but not limited to epithelial cancers such as pancreatic, lung, cervical, ovarian, prostate, non-small cell lung carcinomas, melanomas, squamous cell cancers, etc. It is contemplated that the present methods described herein may find use in prognosis and predicting patient response to certain PARP combination therapies that may be used in various cancer treatments for multiple types of cancers so long as the biomarker predictor panel described herein and the patient criteria described herein is present as identifying a patient suitable for such combination therapy.

It is contemplated that patients with different types of cancers can be evaluated using the present methods including but not limited to, breast cancer, non small cell lung carcinoma, ovarian, endometrial, prostate, epithelial cancers, melanoma, etc.

In other embodiments, a computer-readable medium or computer software comprising instructions to perform one or more steps as described in the process below or exemplified in the Matlab codes provided below. The software may comprise instructions to output (e.g., display, play, print or store) the biomarkers predicted or selected. The steps can be as outlined below in the code at the lines beginning with a “%” symbol.

Thus in one embodiment a computer system to implement the algorithm and methods described. Such a computer system can comprise code for interpreting the results of an expression analysis evaluating the level of expression of the 6-8 panel genes or code for interpreting the results of an expression analysis evaluating the level of expression of the 6-8 panel genes. Thus in an exemplary embodiment, the expression analysis results are provided to a computer where a central processor executes a computer program for determining the biomarker selection, expression levels, validation and/or predicted response.

In some embodiments the use of a computer system, such as that described above, which comprises: (1) a computer; (2) a stored bit pattern encoding the expression results obtained by the methods of the invention, which may be stored in the computer; and, optionally, (3) a program for determining the predicted response.

In another embodiment, methods of generating a report based on the detection of gene expression products for a cancer patient that is evaluated for their predicted sensitivity or resistance profile to PARP inhibitors. Such a report is based on the detection of gene expression products encoded by the 6-8 genes identified in the 6-8 biomarker panels, or detection of gene expression products encoded by the 6-8 genes in the 6-8 gene biomarker panels.

Various embodiments of algorithms and software as described herein in the Examples can be implemented in the form of logic in software, firmware, hardware, or a combination thereof. The logic may be stored in or on a machine-accessible memory, a machine-readable article, a tangible computer readable medium, a computer-readable storage medium, or other computer/machine-readable media as a set of instructions adapted to direct a central processing unit (CPU or processor) of a logic machine to perform a set of steps that may be disclosed in various embodiments of an invention presented within this disclosure. The logic may form part of a software program or computer program product as code modules become operational with a processor of a computer system or an information-processing device when executed to perform a method or process in various embodiments of an invention presented within this disclosure. Based on this disclosure and the teachings provided herein, a person of ordinary skill in the art will appreciate other ways, variations, modifications, alternatives, and/or methods for implementing in software, firmware, hardware, or combinations thereof any of the disclosed operations or functionalities of various embodiments of one or more of the presented inventions.

Once the expression levels of the 6, 7 and/or 8 identified biomarkers in a patient are determined by the present methods, a clinician may provide a prognosis based upon the predicted patient response to certain PARP therapies. For example, as determined by the prescribed methods, after (a) measuring the amplification or expression level of at least one gene up to all the genes selected from the group of genes encoding BRCA1, H2AFX, MRE11A, TDG, XRCC5, BRCA2, CHEK1, CHEK2, MK2, NBS1 and XPA in a sample from the patient; and (b) comparing the amplification or expression level of the gene(s) from the patient with the amplification or expression level of the gene in a normal tissue sample or a reference amplification or expression level, the predicted response of the patient to a PARP inhibitor is determined. An increase of amplification or expression of one gene selected from the group consisting of the genes encoding BRCA1, H2AFX, MRE11A, TDG, XRCC5, NBS1 and XPA, and/or a decrease of amplification or expression of one gene selected from the group consisting of the genes encoding BRCA2, CHEK1, CHEK2 and MK2 indicates the patient is resistant to a PARP inhibitor. If an decrease of amplification or expression of one gene selected from the group consisting of the genes encoding BRCA1, H2AFX, MRE11A, TDG, XRCC5, NBS1 and XPA, and/or a increase of amplification or expression of one gene selected from the group consisting of the genes encoding BRCA2, CHEK1, CHEK2 and MK2 was detected, such determination indicates the patient is sensitive to a PARP inhibitor. In some embodiments, a report can be generated or an electronic medical record is changed or altered. In some embodiments, based upon the predicted resistance or sensitivity response of the patient, a clinician can institute or alter the therapeutic regimen of a patient, prescribe a PARP inhibitor or combination therapy, or a non-PARP inhibitor or therapy.

In some embodiments of the invention, the method further comprises administering a therapeutically effective amount of the PARP inhibitor to the patient. Compounds and formulations of PARP inhibitors that may be suitable for use in the present invention, and the dosages and methods of administration thereof are known by clinicians. Some examples are taught in U.S. Pat. Nos. 8,071,579; 8,071,623; 7,732,491; 7,151,102; 7,196,085; 7,407,957; 7,449,464; 7,750,006; and 7,981,889, hereby incorporated by reference. Known PARP inhibitors include but are not limited to, compounds such as 3-amino benzamide, benzimidazaoles, phthalazinones, quinolinones, quinoxalinones, benzamide-4-carboxmides, Olaparib (AstraZeneca), ABT-888 (Abbott Laboratories), Iniparib (BiPar Sciences/Sanofi-aventis), AG014699 (Pfizer Inc.), INO-1001 (Inotek/Genentech), MK-4827 (Merck), CEP-8933/CEP-9722 (Cephalon), and GPI 21016 (MGI Pharma).

### Example 1

### Determining an Eight-Biomarker Predictor Panel

Thirty-three in vitro breast cancer cell lines were administered the PARP inhibitor Olaparib, with sensitivity to the compound summarized as the dose necessary to kill 50% of each culture. mRNA expression (Affymetrix U133A, Exon 1.0ST array) and transcriptome sequence (Illumina GAII) were available for 22/33 cell lines, among which 9 were sensitive and 13 resistant. To obtain robust predictive markers that are minimally dependent on the specific PARP inhibitor and expression platform, a bottom-up approach was opted for, restricted to genes in the major DNA repair pathways. Logistic regression with forward selection was used to determine the most important markers, further reduced based on consistency across platforms. The weighted voting algorithm was used to build the final predictor. Eight U133A and two U133 plus 2 data sets with number of tumor samples varying from 61 to 289, 117 samples from I-SPY1 with U133A data, and 430 TCGA samples with custom Agilent 244K gene expression were subsequently used to verify prevalence, to identify the subpopulations that are likely to respond according to the predictor, and to determine cross-platform generalizability.

Results: Response to Olaparib showed moderate subtype specificity with basal subtype more sensitive and luminal subtype more resistant (one-way ANOVA, p-value 0.284). An association was observed between BRCA1 mutation and drug sensitivity, with mutated cell lines more sensitive (p-value 0.037) with a lower BRCA1 expression (p-value 0.048) and copy number (p-value 0.012). For the development of a genomic signature that might work for multiple PARP inhibitors and expression platforms, prior knowledge of DNA repair pathways was incorporated and stringent criteria for marker inclusion were applied using three different platforms. Eight genes fulfilled the criteria, of which 5 were resistance markers and 3 sensitivity markers. When testing the 8-gene signature in ten U133A/plus 2 data sets, 40-48% of patients were predicted to be responsive to Olaparib. Application of this classifier to I-SPY1 tumor data revealed that 41% of patients are likely to respond to Olaparib, with a bias toward the basal, luminal A and ERBB2-negative subtypes. Prevalence and subtype association were confirmed in 430 samples on a distinct platform (Agilent).

Discussion.

Biomarkers from literature that were found to be significant in our cell line panel are the following: BRCA1 mutation, with mutated cell lines more sensitive to Olaparib compared to the wildtype cell lines; BRCA1 deletion, with lower copy number in sensitive with respect to resistant cell lines; down-regulation of APEX1, AURKA, BRCA1, EMSY, ESR1, FANCD2, 2H2AX, MRE11A, PGR, and TNKS2, and up-regulation of CDK5, CHEK2, HMGA1, STK22C, and XRCC3 in sensitive with respect to resistant cell lines

Cell line exposure to Olaparib has yielded a DNA pathway-based 8-gene predictive signature, observed in a substantial fraction of primary breast tumors predicted to benefit from Olaparib. Depending on the validation data set, 40-48% of patients were predicted to respond to Olaparib. Association with subtype for I-SPY1 and TCGA revealed that Olaparib responding tumors might include the basal, luminal A and ERBB2-negative subtypes.

In a later stage, the set of 8 markers will be retrospectively validated on tissue samples prospectively collected in the I-SPY2 trial from patients treated with ABT-888. Because various PARP inhibitors have different effects and levels of specificity for BRCA mutation carriers, predictors that work for one PARP inhibitor might not necessarily work for another PARP inhibitor. The suggested cell line based predictor of response to Olaparib will therefore be refined and further optimized in I-SPY2 for ABT-888. The regimen of PARP inhibition with associated predictive biomarkers might subsequently graduate into phase 3 studies.

A typical problem in biomarker discovery is the limited statistical power due to the large number of gene expression levels measured for a small set of samples. In our study, expression data on thousands of genes were available for 22 cell lines. The “large p, small n” problem, however, was circumvented with a bottom-up approach, thereby restricting the focus on a reduced set of 118 genes from 6 principal DNA repair pathways. An inherent weakness of our breast cancer cell line panel is that the three BRCA1-mutated cell lines are all sensitive to Olaparib, whilst none of the cell lines are BRCA2-mutated.

### Materials and Methods.

Drug Response Data.

For measurement of sensitivity to KU0058948 (Olaparib; KuDOS Pharmaceuticals/AstraZeneca), exponentially growing cells were seeded in six-well plates at a concentration of 5,000 cells per well. Cells were exposed continuously to the inhibitor, and medium and inhibitor were replaced every four days. After 15 days, cells were fixed and stained with sulphorhodamine-B (Sigma, St. Louis, USA) and a colorimetric assay performed as described previously [8]. Surviving fractions (SFs) were calculated and drug sensitivity curves determined with the Four Parameter Logistic Regression model as previously described [7].

Molecular Data of Breast Cancer Cell Lines.

DNA extracted from cell lines was labeled and hybridized to the Affymetrix Genome-Wide Human SNP Array 6.0 for DNA copy number. Data were segmented using the circular binary segmentation (CBS) algorithm from the Bioconductor package DNAcopy [73], followed by summarization at gene level with the R package CNTools. Human genome build 36 was used for processing and annotating. Gene expression data for the cell lines were derived from Affymetrix GeneChip Human Genome U133A and Affymetrix GeneChip Human Exon 1.0 ST arrays. U133A data was preprocessed with RMA in R, but with use of two distinct annotation files: standard annotation by Affymetrix followed by selection of the maximal varying probe set per gene, and a custom annotation to gene level [74]. For the exon array, an improved mapping of the probes to human genome build 36.1 obtained by TCGA was used [60]. Whole transcriptome shotgun sequencing (RNA-seq) was completed on breast cancer cell lines and expression analysis was performed with the ALEXA-seq software package as previously described [75]. The Illumina Infinium Human Methylation27 BeadChip Kit was used for the genome-wide detection of the degree of methylation at 27,578 CpG loci, spanning 14,495 genes, with genome build 36 for annotation [98]. At each single CpG locus, degree of methylation is measured through M and U probes that differ at the C for each CpG dinucleotide and allow measuring the abundance of methylated and unmethylated DNA, respectively. These values are reliable when the number of CG dinucleotides and off-CpG cytosines both exceed 2. Cross-hybridization might occur when the number of CpG dinucleotides is too low. At least 3 C's outside of a CpG dinucleotide in addition guarantees good specificity to successfully bisulfite converted DNA, thereby not misinterpreting unconverted DNA as methylated DNA. Reverse protein lysate array (RPPA) is an antibody-based method to quantitatively measure protein abundance [76] and was used for the measurement of 146 (phospho)proteins. Mutation data was extracted from COSMIC v53, the catalogue of somatic mutations in cancer [Forbes S A, Bhamra G, Bamford S, Dawson E, Kok C, Clements J, Menzies A, Teague J W, Futreal P A, Stratton M R: The Catalogue of Somatic Mutations in Cancer (COSMIC). Curr Protoc Hum Genet. 2008, Chapter 10:Unit 10 11] (as of May 18, 2011). Finally, siRNA data for 714 kinases and kinase-related genes were generated in triplicate as previously described [51]. The average was taken across these triplicates as well as the 1 to 4 probes targeting each individual gene. We refer to Heiser et al. [(2012) Subtype and pathway specific responses to anticancer compounds in breast cancer. Proceedings of the National Academy of Sciences of the United States of America 109 (8):2724-2729] for a detailed description of the preprocessing of all molecular data sets.

Validation Data.

U133A, U133B and U133 plus 2 expression data for 10 tumor sets (with Gene Expression Omnibus IDs GSE2034, GSE20271, GSE23988, GSE4922, GSE1456, GSE7390, GSE11121, GSE12093, GSE23177, GSE5460) were preprocessed with RMA in R with use of Affymetrix's standard annotation. Also the U133A expression data of 117 tumor samples from the I-SPY1 clinical trial were preprocessed with RMA. Custom Agilent 244K expression data at gene level was available for 430 breast invasive carcinoma samples collected by TCGA (The Cancer Genome Atlas) as of Jun. 3, 2011 [The Cancer Genome Atlas Data Portal, available at TCGA website tcga-data.nci.nih.gov/tcga/tcgaHome2.jsp]. Missing values in this data set were imputed with KNNimputer in R [Troyanskaya O, Cantor M, Sherlock G, Brown P, Hastie T, Tibshirani R, Botstein D, Altman RB: Missing value estimation methods for DNA microarrays. Bioinformatics 2001, 17(6):520-525]. All expression data sets were median normalized per gene across all samples.

The TCGA and I-SPY1 tumor samples were subtyped with PAM50, a 50-gene set introduced for standardizing the categorical classification of breast cancer subtype into luminal A, luminal B, basal, ERBB2-amplified and normal-like [Parker J S, Mullins M, Cheang M C, Leung S, Voduc D, Vickery T, Davies S, Fauron C, He X, Hu Z et al: Supervised risk predictor of breast cancer based on intrinsic subtypes. J Clin Oncol 2009, 27(8):1160-1167]. The normal-like samples were excluded from the association study of response prediction to Olaparib with subtype.

Statistical Analyses.

The Wilcoxon rank sum test was used for validation of biomarkers from literature in the cell line panel. The chi-square test was used for the association of breast cancer subtype with response prediction to Olaparib. All analyses were performed in Matlab R2010b for Mac.

Biomarker Selection and Model Building.

Logistic regression (LR) with forward selection (5-fold CV) was opted for and applied to each DNA repair pathway separately. Genes that resulted in the best data fit were consecutively added. The difference in fit value when incorporating an additional gene was modeled with a chi-square distribution. When the gain in data fit was not significantly different from zero, no genes were further added to the logistic regression model as not significantly improving the discriminatory power. LR model building was repeated 100 times to determine the most important markers selected in over half of the iterations. These markers were further reduced to those selected with consistent pattern of sensitivity for at least 2 out of 3 platforms (U133A with standard or custom annotation, exon array and RNA-seq).

The weighted voting algorithm [Moulder S, Yan K, Huang F, Hess K R, Liedtke C, Lin F, Hatzis C, Hortobagyi G N, Symmans W F, Pusztai L: Development of candidate genomic markers to select breast cancer patients for dasatinib therapy. Molecular cancer therapeutics 2010, 9(5):1120-1127] was used to build the predictor. For each gene g, the median μ and standard deviation a of its median-normalized expression levels were calculated for the class of sensitive and resistant cell lines separately. The weight w_{g} and decision boundary b_{g} for gene g follows from

w_{g}=[μ₁(g)−μ₂(g)]/[σ₁(g)−σ₂(g)],

b_{g}=[μ₁(g)+μ₂(g)]/2.

The weights w_{g} and decision boundaries b_{g} for the 8 genes were obtained from the median-centered U133A expression cell line data, preprocessed with RMA with use of the standard annotation from Affymetrix.

For the calculation of predicted probability of response to PARP inhibition for a new set of tumor samples, the expression data at logarithmic scale are median normalized for each gene g across all samples (X_{g}). The assignment of a new sample to the class of responders or non-responders follows from the sign of the sum of weighted votes across the set of biomarkers. For each individual biomarker g, the weighted vote V_{g} for a sample is calculated by subtracting the boundary value b_{g} from the gene expression value X_{g}, followed by multiplication of this difference with the biomarker weight w_{g} derived from the cell line data. A positive value for the weighted vote indicates that this sample is assigned to the class of responders according to the individual biomarker, and a negative value indicates a vote for the class of non-responders. After calculation of the weighted vote for all biomarkers, the positive votes are summed, resulting in the total weighted vote for the class of responders (V₁), whilst the sum of the negative votes represents the total weighted vote for the class of non-responders (V₂). The sign of the difference S in total weighted vote between both classes determines the class the sample is assigned to, with the absolute value of the difference being an indication for the confidence of the class prediction.

$${X}_{8} = median - normalized log expression level of gene g in a new sample$$

$$Weighted vote for gene g \text{:} {V}_{g} = {w}_{g} [ {X}_{g} - {b}_{g} ]$$

$$Total weighted vote for class 1 \text{:} {V}_{1} = {\sum }_{g}^{ } {V}_{g} {I}_{1}$$

$$with$$

$${I}_{1} = 1 if {V}_{g} > 0 , \\ 0 otherwise$$

$$Total weighted vote for class 2 \text{:} {V}_{2} = {\sum }_{g}^{ } {V}_{g} {I}_{2}$$

$$with$$

$${I}_{2} = 1 if {V}_{g} < 0 , \\ 0 otherwise$$

$$Difference score \text{:} S = {V}_{1} - \vert {V}_{2} \vert$$

Probability of Response.

The sign of the difference S in total weighted vote between both classes determines the class the sample is assigned to, with the absolute value of the difference being an indication for the confidence of the class prediction.

Difference score: S=V₁−|V₂|

Signature Validation.

Co-expression patterns between cell lines and tumor samples were investigated with use of the correlation-based coherence matrix and the Jaccard similarity coefficient [72]. Coherence matrices were generated for the cell line panel and validation data sets separately. The Jaccard coefficient is defined as the number of gene pairs with the same correlation pattern in both coherence matrices divided by the total number of gene pairs (only considering one triangular part of the matrix). This coefficient ranges from 0 to 1, with values closer to 1 representing better similarity.

Tumor Data Normalization.

When applying the 8-gene signature to tumor samples, the same probe sets as in the cell line panel should be used in case of Affymetrix U133A or U133 plus 2 data; otherwise expression data at gene level. After preprocessing of the tumor data set specific for the used platform (e.g. RMA in R for Affymetrix expression data), tumor data should be presented at logarithmic scale, followed by median normalization of each gene across all samples (that is, subtraction of the median expression of each gene across all samples from the data).

Conclusion:

Cell line exposure to Olaparib has yielded an 8-gene predictor of sensitivity. This signature was observed in a substantial fraction of the I-SPY population and primary breast tumors predicted to benefit from Olaparib, and will therefore prospectively be tested in I-SPY2 for PARP inhibitor ABT-888 in non-ERBB2+ patients.

### Example 2

### Determining Patient Response to PARP Inhibition Using an Eight-Biomarker Predictor Panel

A patient biopsy is obtained from a patient having diagnosed with breast cancer. The amplification and expression levels of BRCA1, BRCA2, H2AFX, MRE11A, TDG, XRCC5, CHEK1 or CHEK2 are obtained from the sample and a determination is made whether the patient would be resistant or sensitive to a PARP inhibitor such as Olaparib. The patient's therapy could be altered to recommend non-use of PARP inhibitors if the patient is determined to be resistant or if the patient is determined to be sensitive to PARP inhibitors, then PARP inhibitors are prescribed and administered.

### Example 3

### Determining a Seven-Biomarker Predictor Panel

We identified candidate biomarkers associated with response to olaparib by correlating responses to 9 concentrations of olaparib in a panel of well characterized breast cancer cell lines with the transcription levels of genes involved in aspects of DNA repair. Genes tested for correlation with olaparib response included those reported in the literature to be directly relevant to PARP inhibitor response or involved more generally in some aspect of DNA repair (FIG. 1). We applied this signature to primary tumor data to identify the frequency and characteristics of tumors that might be expected to respond to olaparib. These studies set the stage for a clinical test of the sensitivity and specificity of this predictor and indicate known subtypes of breast cancers that might be preferentially sensitive to olaparib.

### Material and Methods

Breast Cancer Cell Lines, Assay, and Molecular Data.

The sensitivity of a panel of 22 breast cancer cell lines to KU0058948 (olaparib; KuDOS Pharmaceuticals/AstraZeneca) was measured with a growth inhibition assay [Farmer H, McCabe N, Lord C J, Tutt A N, Johnson D A, Richardson T B, Santarosa M, Dillon K J, Hickson I, Knights C et al: Targeting the DNA repair defect in BRCA mutant cells as a therapeutic strategy. Nature 2005, 434(7035):917-921, Edwards S L, Brough R, Lord C J, Natrajan R, Vatcheva R, Levine D A, Boyd J, Reis-Filho J S, Ashworth A: Resistance to therapy caused by intragenic deletion in BRCA2. Nature 2008, 451(7182):1111-1115]. The following molecular data were collected for the panel: copy number (Affymetrix SNP6), gene expression (Affymetrix U133A, Affymetrix Exon 1.0 ST), transcriptome sequencing (Illumina GAII), methylation (Illumina Methylation27), protein abundance (reverse protein lysate array), and mutation status (COSMIC, [Weigelt B, Warne P H, Downward J (2011) PIK3CA mutation, but not PTEN loss of function, determines the sensitivity of breast cancer cells to mTOR inhibitory drugs. Oncogene 30 (29):3222-3233. doi:10.1038/one.2011.421). A detailed description of the availability and preprocessing of all molecular data sets is provided below and [Heiser L M, et al., (2012) Subtype and pathway specific responses to anticancer compounds in breast cancer. Proceedings of the National Academy of Sciences of the United States of America 109 (8):2724-2729. doi:10.1073/pnas.1018854108].

Statistical Analyses.

The Wilcoxon rank sum test was used to test the association of drug response with individual biomarkers. Drug response was associated with subtype, triple negativity and mutation status with use of the Fisher's exact test. Due to the small sample size, a p-value <0.05 was deemed significant, whilst a p-value <0.1 was considered a trend. Logistic regression (LR) with forward feature selection (5-fold CV) was used to identify candidate biomarkers and was applied to each DNA repair pathway separately. The resulting biomarkers were combined into a predictor using a weighted voting algorithm [Moulder S, Yan K, Huang F, Hess K R, Liedtke C, Lin F, Hatzis C, Hortobagyi G N, Symmans W F, Pusztai L: Development of candidate genomic markers to select breast cancer patients for dasatinib therapy. Molecular cancer therapeutics 2010, 9(5):1120-1127]. The Matlab code below was used for signature development and validation. A chi-square test was used to test for associations of breast cancer subtype with response to olaparib.

### Results

Olaparib Response in a Panel of 22 Breast Cancer Cell Lines.

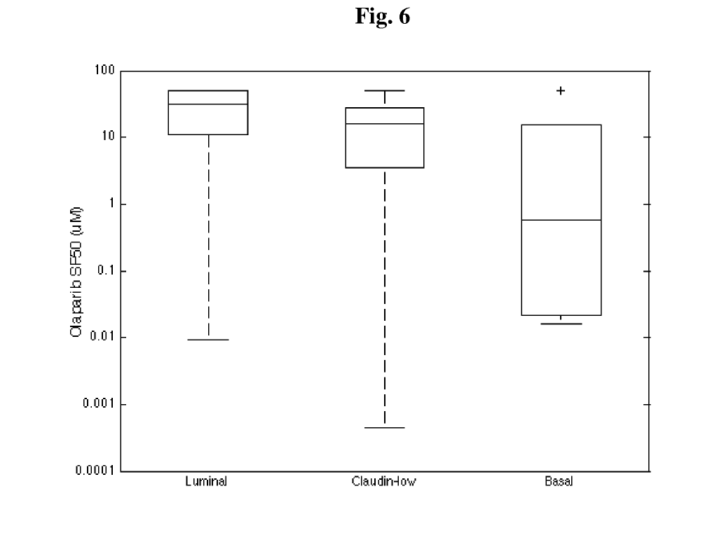

Twenty-two breast cancer cell lines previously profiled for RNA transcript levels were tested for response to 9 concentrations of olaparib (see Table 8). These cells mirror many of the transcriptional and genomic characteristics of primary breast tumors and have been used to model responses to a large number of experimental and approved therapeutic compounds [Neve R M, Chin K, Fridlyand J, Yeh J, Baehner F L, Fevr T, Clark L, Bayani N, Coppe J P, Tong F et al: A collection of breast cancer cell lines for the study of functionally distinct cancer subtypes. Cancer cell 2006, 10(6):515-527, Heiser, L. et al. (2012) Subtype and pathway specific responses to anticancer compounds in breast cancer. Proceedings of the National Academy of Sciences of the United States of America 109 (8):2724-2729. doi:10.1073/pnas.1018854108]. The concentration of olaparib needed to reduce survival to 50% (SF50) was used as a quantitative measure of sensitivity and ranged from 0.44 nM to 32 μM. The SF50 was not reached for 5 cell lines at the maximum treatment concentration of 50 μM olaparib. Olaparib response obtained with the growth inhibition assay was not influenced by growth rate assessed as doubling time (Spearman correlation coefficient −0.036, p-value 0.874). FIG. 2 shows the waterfall plot of SF50 with cell lines ordered from most resistant at the left to most sensitive at the right. Cell lines were divided into a group of 15 resistant and 7 sensitive cell lines, based on an SF50 threshold of 1 μM. Drug response was not significantly associated with breast cancer subtype (p-value luminal vs. basal 0.136; FIG. 6), and did not differ between ERBB2 amplified and non-ERBB2 amplified cell lines (p-value 1), with transcriptional subtypes assigned to cell lines as previously reported [88]. Four of the 7 sensitive cell lines (57%) were triple negative, compared to 5 of 15 (33%) resistant cell lines (p-value 0.376). Table 9 summarizes characteristics for the 22 cell lines, with SF50, doubling time, transcriptional ER, PR and ERBB2 status, and the molecular data available for each of them.

Molecular Features Involved in DNA Repair Associate with Olaparib Response.

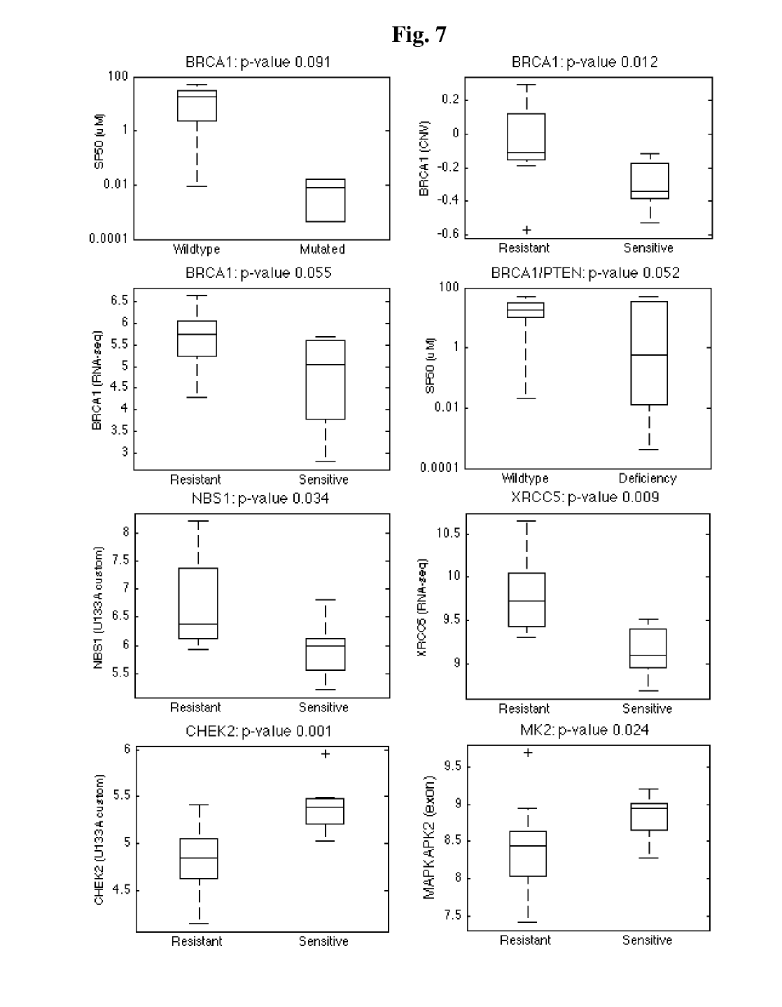

We selected candidate molecular features that might be developed as biomarkers for prediction of response to olaparib as those features involved in DNA repair activities that were associated with quantitative response to olaparib in the cell line panel. Molecular features included pretreatment RNA transcript levels, mutation status, copy number variation and promoter methylation status. Specific genes tested involved aspects of DNA repair listed by Wang and Weaver [Wang X, Weaver D: The ups and downs of DNA repair biomarkers for PARP inhibitor therapies. Am J Cancer Res 2011, 1(3):301-327]; ER, PR and ERBB2 due to the importance of PARP inhibition for triple negative breast cancer [Plummer R: Poly(ADP-ribose) polymerase inhibition: a new direction for BRCA and triple-negative breast cancer? Breast cancer research: BCR 2011, 13(4):218]; and PARP family members PARP1, PARP2, VPARP, TNKS and TNKS2. This approach is based on observations that in vitro models showing high sensitivity to PARP inhibitors often have BRCA and PTEN deficiencies [Farmer H, McCabe N, Lord C J, Tutt A N, Johnson D A, Richardson T B, Santarosa M, Dillon K J, Hickson I, Knights C et al: Targeting the DNA repair defect in BRCA mutant cells as a therapeutic strategy. Nature 2005, 434(7035):917-921, Mendes-Pereira A M, Martin S A, Brough R, McCarthy A, Taylor J R, Kim J S, Waldman T, Lord C J, Ashworth A: Synthetic lethal targeting of PTEN mutant cells with PARP inhibitors. EMBO molecular medicine 2009, 1(6-7):315-322], copy number variations involving BRCA1 and PARP1 [Holstege H, Horlings H M, Velds A, Langerod A, Borresen-Dale A L, van de Vijver M J, Nederlof P M, Jonkers J: BRCA1-mutated and basal-like breast cancers have similar aCGH profiles and a high incidence of protein truncating TP53 mutations. BMC cancer 2010, 10:654] and/or hypermethylation of the promoter regions of genes BRCA1 and FANCF [Turner N C, Ashworth A: Biomarkers of PARP inhibitor sensitivity. Breast cancer research and treatment 2011, 127(1):283-286]. Molecular features showing statistically significant associations with SF50 values are summarized in Table 14 and illustrated in FIG. 7.

The transcription levels of MRE11A, NBS1, TNKS, TNKS2, XPA and XRCC5 were significantly lower (p<0.05; fold-change>2) in the sensitive compared to the resistant cell lines for at least one expression platform (U133A, exon array and RNA-seq), whilst transcription levels for BRCA1, ERCC4, FANCD2 and PR tended to be lower in sensitive lines (p<0.1). We refer to Table 14a for the list of significant associations per platform. PR protein levels measured using reverse phase protein lysate arrays [76] were also significantly reduced in the sensitive cell lines (p<0.05). Transcript levels for CHEK2 and MK2 were significantly higher in the sensitive compared to the resistant lines (p<0.05), with a similar trend for PARP2 and XRCC3 (p<0.1). Although PARP1 has been shown to be overexpressed in 58% of invasive breast cancer samples [Goncalves A, Finetti P, Sabatier R, Gilabert M, Adelaide J, Borg J P, Chaffanet M, Viens P, Birnbaum D, Bertucci F: Poly(ADP-ribose) polymerase-1 mRNA expression in human breast cancer: a meta-analysis. Breast cancer research and treatment 2011, 127(1):273-281] and upregulated at protein level in 82% of BRCA1-associated breast cancer samples [30], there is no consensus on its importance as a biomarker of response to PARP inhibitors [Cotter M, Pierce A, McGowan P, Madden S, Flanagan L, Quinn C, Evoy D, Crown J, McDermott E, Duffy M: PARP1 in triple-negative breast cancer: expression and therapeutic potential. J Clin Oncol 2011, 29(15_suppl):1061, Zaremba T, Ketzer P, Cole M, Coulthard S, Plummer E R, Curtin N J: Poly(ADP-ribose) polymerase-1 polymorphisms, expression and activity in selected human tumour cell lines. British journal of cancer 2009, 101(2):256-262]. In our cell line panel, expression of PARP1 mRNA levels were not significantly higher in the sensitive lines compared to the resistant lines (median p-value 0.277) (Table 14a).

The BRCA1-mutated cell lines MDAMB436 and SUM149PT had a trend to be more sensitive to olaparib compared to the wild-type cell lines (p-value 0.091) (Table 14b). Likewise, cells with reduced BRCA1 copy number were significantly more sensitive to olaparib than cells with normal copy number at this locus (p-value 0.012) (Table 14c). PTEN loss of function, which was defined as mutation and/or lack of expression, was not significantly associated with olaparib SF50 response (p-value 0.145), even though previous studies from our group suggested that PTEN deficiency can cause olaparib sensitivity [Mendes-Pereira A M, et al.: Synthetic lethal targeting of PTEN mutant cells with PARP inhibitors. EMBO molecular medicine 2009, 1(6-7):315-322; Dedes K J, et al: PTEN deficiency in endometrioid endometrial adenocarcinomas predicts sensitivity to PARP inhibitors. Science translational medicine 2010, 2(53):53ra75]. Lack of association in the cell line panel could be ascribed to the small sample size and/or to the possibility that the univariate associations do not take into account important multigene effects. Since BRCA1 mutations have been associated with reduced PTEN expression [Saal L H, Gruvberger-Saal S K, Persson C, Lovgren K, Jumppanen M, Staaf J, Jonsson G, Pires M M, Maurer M, Holm K et al: Recurrent gross mutations of the PTEN tumor suppressor gene in breast cancers with deficient DSB repair. Nature genetics 2008, 40(1):102-107], we tested for association of either BRCA1 mutation or PTEN deficiency with olaparib sensitivity. We found that cell lines with a deficiency in either gene tended to be more sensitive to olaparib than cell lines with functional BRCA1 and PTEN (p-value 0.052) (Table 14b). No association was found between TP53 mutation status and drug response (p-value 0.376).

Cell Line-Based 7-Transcript Signature Predicts Response to Olaparib.

We used a breast cancer cell line panel comprised of luminal, basal and claudin-low cell lines to develop a multi-transcript predictor of sensitivity to olaparib according to the REMARK recommendations [89]. We limited the predictor to transcript levels to facilitate clinical application. We considered all breast cancer subtypes for the development of the predictor based on a study of RAD51 focus formation in cells responding to a PARP inhibitor. That study showed that 30 to 40% of triple negative breast cancers appeared not to have defective HR and therefore might not benefit from a PARP inhibitor whilst ˜20% of non-triple negative breast cancers appeared to have defective HR and therefore might respond to a PARP inhibitor [90]. Thus, we reasoned that a predictor developed using the complete cell line panel might be applicable to the full spectrum of breast cancer covered by the cell line panel. As shown in FIG. 1, the molecular features tested as candidate biomarkers were limited to genes involved in DNA repair pathways BER, NER, MMR, HR/FA, NHEJ and DDR as defined by Wang and Weaver [Wang X, Weaver D: The ups and downs of DNA repair biomarkers for PARP inhibitor therapies. Am J Cancer Res 2011, 1(3):301-327] and in the Kyoto Encyclopedia of Genes and Genomes (KEGG) database release 55.1 [Kanehisa M, Goto S, Furumichi M, Tanabe M, Hirakawa M: KEGG for representation and analysis of molecular networks involving diseases and drugs. Nucleic acids research 2010, 38(Database issue):D355-360. This led to the selection of 118 genes (see Table 15) that were tested for association between transcript levels and response to olaparib. These transcript levels were measured using three different mRNA analysis platforms (Affymetrix U133A arrays, Affymetrix exon arrays and Illumina RNA-seq).

We identified the most important transcripts by applying logistic regression with forward feature selection (5-fold CV) 100 times. Markers significantly associated with olaparib response in over half of the iterations are shown in Table 10. These were further reduced to 7 gene transcripts that were significantly associated with olaparib response in all three mRNA analysis platforms. Five transcript levels (candidate resistance markers BRCA1, MRE11A, NBS1, TDG and XPA) were inversely associated with predicted probability of response and 2 transcript levels (candidate sensitivity markers CHEK2 and MK2) were positively associated with predicted probability of response. BRCA1 is involved in DSB repair via RAD51-mediated HR [Gudmundsdottir K, Ashworth A: The roles of BRCA1 and BRCA2 and associated proteins in the maintenance of genomic stability. Oncogene 2006, 25(43):5864-5874; Tutt A, Ashworth A: The relationship between the roles of BRCA genes in DNA repair and cancer predisposition. Trends in molecular medicine 2002, 8(12):571-576]. CHEK2 is a kinase with signal transduction function in cell cycle regulation and checkpoint responses [Sancar A, Lindsey-Boltz L A, Unsal-Kacmaz K, Linn S: Molecular mechanisms of mammalian DNA repair and the DNA damage checkpoints. Annual review of biochemistry 2004, 73:39-85], and is involved in the major parallel DDR pathway ATM-CHEK2 [Wang X, Weaver D: The ups and downs of DNA repair biomarkers for PARP inhibitor therapies. Am J Cancer Res 2011, 1(3):301-327]. CHEK2 has also been reported as an intermediate-level breast cancer risk gene, regardless of family history [CHEK2 Breast Cancer Case-Control Consortium (2004) CHEK2*1100delC and susceptibility to breast cancer: a collaborative analysis involving 10,860 breast cancer cases and 9,065 controls from 10 studies. American journal of human genetics 74 (6):1175-1182. doi:10.1086/421251; Fletcher O, et al., (2009) Family history, genetic testing, and clinical risk prediction: pooled analysis of CHEK2 1100delC in 1,828 bilateral breast cancers and 7,030 controls. Cancer epidemiology, biomarkers & prevention: a publication of the American Association for Cancer Research, cosponsored by the American Society of Preventive Oncology 18 (1):230-234. doi:10.1158/1055-995.EPI-08-0416]. Besides the standard DDR pathways, the cell-cycle checkpoint pathway p38MAPK/MK2 is additionally activated in TP53 mutant cells [Reinhardt H C, Aslanian A S, Lees J A, Yaffe M B (2007) p53-deficient cells rely on ATM- and ATR-mediated checkpoint signaling through the p38MAPK/MK2 pathway for survival after DNA damage. Cancer cell 11 (2):175-189. doi:10.1016/j.ccr.2006.11.024]. MK2 activity is critical for prolonged checkpoint maintenance through a process of posttranscriptional regulation of gene expression [Reinhardt H C, Hasskamp P, Schmedding I, Morandell S, van Vugt M A, Wang X, Linding R, Ong S E, Weaver D, Carr S A, Yaffe M B (2010) DNA damage activates a spatially distinct late cytoplasmic cell-cycle checkpoint network controlled by MK2-mediated RNA stabilization. Molecular cell 40 (1):34-49. doi:10.1016/j.molcel.2010.09.018]. MRE11A and NBS1 are part of the MRN complex, a multifaceted molecular machine for DSB recognition [Williams G J, Lees-Miller S P, Tainer J A: Mre11-Rad50-Nbs1 conformations and the control of sensing, signaling, and effector responses at DNA double-strand breaks. DNA repair 2010, 9(12):1299-1306]. Finally, TDG is part of the BER pathway, whilst XPA encodes a zinc finger protein that is part of the NER complex.

We combined information on the 7 transcript levels to form a predictive signature using a weighted voting algorithm as described further below and in Heiser L, et al, (2012) Subtype and pathway specific responses to anticancer compounds in breast cancer. Proceedings of the National Academy of Sciences of the United States of America 109 (8):2724-2729. doi:10.1073/pnas.1018854108, and hereby incorporated by reference. This algorithm assigns a weight and decision boundary to each of the 7 genes, based on their expression distribution for the class of sensitive vs. resistant cell lines (see Table 11). For this signature to work on external samples, the transcript levels were normalized to the geometric mean of seven control genes, followed by median normalization across the cell lines. The larger the weight for a gene transcript level, the more influence this gene has on predicted probability of response. Positive weights were assigned for sensitivity markers and negative weights were assigned for resistance markers.

Prevalence of 8-21% of Predicted Responding Patients, with Trend Towards the Basal subtype.

We analyzed expression profiles measured for breast cancer patients not treated with PARP inhibitors to understand which patients would have a likelihood of response to olaparib according to our 7-transcript predictor. We used seven U133A and one U133 plus 2 data sets on 1,846 primary breast tumors with or without metastasis, heterogeneous in treatment and ER/PR/LN status. Our 7-transcript response algorithm predicted that 8-21% of patients in the 8 data sets would be responsive to olaparib (Table 12), using threshold 0.0372 obtained from the cell lines to distinguish sensitive from resistant. The fraction predicted to respond was inversely related to the fraction of ER-positive patients in each data set (Pearson correlation coefficient −0.614, p-value 0.1). We also tested the 7-transcript predictor in Agilent mRNA transcript profiles measured for 536 breast invasive carcinoma samples collected by TCGA (The Cancer Genome Atlas) [The Cancer Genome Atlas Data Portal, available at tcga-data.nci.nih.gov/tcga/tcgaHome2.jsp website]. This required that an Agilent-specific threshold distinguishing sensitive from resistant be established. We accomplished this using a set of Affymetrix and Agilent mRNA transcript profiles measured for 80 I-SPY 1 samples [Hatzis C, et al., (2011) A genomic predictor of response and survival following taxane-anthracycline chemotherapy for invasive breast cancer. JAMA: the journal of the American Medical Association 305 (18):1873-1881; Esserman, L., Breast cancer molecular profiles and tumor response of neoadjuvant doxorubicin and paclitaxel: The I-SPY TRIAL (CALGB 150007/150012, ACRIN 6657). J Clin Oncol 2009, 27(18s):suppl; abstr LBA515]. The Agilent threshold was set so that the fraction of I-SPY 1 samples in the Agilent data set predicted to be sensitive was the same as that predicted to be sensitive using the Affymetrix data. The fraction of samples predicted to be sensitive in the TCGA data set was 12% (Table 12). We assessed the transcriptional subtypes of the patient populations predicted to respond to olaparib in 464 samples from GSE25066 and in 528 TCGA tumor samples after exclusion of the normal-like samples. The tumors predicted to respond were enriched in samples classified as basal-like compared to samples classified as luminal A, luminal B or HER2 (p-value 0.002 and 2.6×10⁻²⁸ for GSE25066 and TCGA, respectively; Table 13).

### Discussion

In this hypothesis generating study, our overall goal was to use quantitative measurements of response to olaparib in 22 breast cancer cell lines to identify molecular features associated with response as a first step towards development of a molecular signature to predict clinical responses. We limited our search for features associated with olaparib response to copy number, DNA sequence abnormalities or transcription levels for 42 genes suggested in [Wang X, Weaver D: The ups and downs of DNA repair biomarkers for PARP inhibitor therapies. Am J Cancer Res 2011, 1(3):301-327] for their association with DNA repair. Molecular features associated with 15 of these 42 genes were found to be significantly associated or to show a trend of association with olaparib response. Specifically, cell lines that were sensitive to olaparib were enriched in BRCA1 mutations or deletions, PARP1 amplification, reduced expression of BRCA1, ERCC4, FANCD2, MRE11A, NBS1, PR, TNKS, TNKS2, XPA and XRCC5 and increased expression of CHEK2, MK2, PARP2 and XRCC3.

Since multiple mechanisms may contribute to olaparib sensitivity, we developed a weighted voting signature to combine influences from multiple markers. We included only transcript levels in our algorithm since most molecular features associated with response were apparent at the transcript level. We limited the search space to molecular features of 118 genes from 6 principal DNA repair pathways in order to increase statistical power. Associations of transcript levels for 118 genes and responses to olaparib for 22 breast cancer cell lines resulted in a 7-gene predictive signature that included 5 resistance markers (BRCA1, MRE11A, NBS1, TDG and XPA) and 2 response markers (CHEK2 and MK2).

The transcript levels of the 7 genes in the predictor were consistent with expectations from the literature. Mutations in BRCA1, loss of heterozygosity at the BRCA1 locus and deregulated expression have been described in literature as potential markers for prediction of response to PARP inhibitors [Turner N, Tutt A, Ashworth A: Hallmarks of ‘BRCAness’ in sporadic cancers. Nature reviews Cancer 2004, 4(10):814-819]. These studies are consistent with our finding that reduced BRCA1 transcript levels are associated with olaparib sensitivity. PARP1 is required for rapid accumulation of MRE11A at DSB sites. Due to the direct interaction between PARP1 and MRE11A, deficiency in MRE11A has been suggested as a mechanism of sensitizing cells to PARP1 inhibition based on the concept of synthetic lethality [Vilar E, Bartnik C M, Stenzel S L, Raskin L, Ahn J, Moreno V, Mukherjee B, Iniesta M D, Morgan M A, Rennert G et al: MRE11 deficiency increases sensitivity to poly(ADP-ribose) polymerase inhibition in microsatellite unstable colorectal cancers. Cancer research 2011, 71(7):2632-2642]. Moreover, a dominant negative mutation in MRE11A in mismatch repair deficient cancers has been shown to sensitize cells to agents causing replication fork stress [Wen Q, Scorah J, Phear G, Rodgers G, Rodgers S, Meuth M: A mutant allele of MRE11 found in mismatch repair-deficient tumor cells suppresses the cellular response to DNA replication fork stress in a dominant negative manner. Molecular biology of the cell 2008, 19(4):1693-1705]. These reports are consistent with our finding that reduced MRE11A transcription is associated with olaparib sensitivity. Experimental disruption of the HR pathway protein NBS1 by RNAi has been reported to increase sensitivity to PARP inhibitors [McCabe N, Turner N C, Lord C J, Kluzek K, Bialkowska A, Swift S, Giavara S, O'Connor M J, Tutt A N, Zdzienicka M Z et al: Deficiency in the repair of DNA damage by homologous recombination and sensitivity to poly(ADP-ribose) polymerase inhibition. Cancer research 2006, 66(16):8109-8115]. This is consistent with our finding that reduced transcription of NBS1 is associated with olaparib sensitivity. Cells with defective NER have been shown to be hypersensitive to platinum agents, with low XPA protein levels in testis tumor cell lines explaining the low capacity to repair cisplatin-induced DNA damage [Koberle B, Masters J R, Hartley J A, Wood R D (1999) Defective repair of cisplatin-induced DNA damage caused by reduced XPA protein in testicular germ cell tumours. Current biology: CB 9 (5):273-276]. PARP inhibitors also enhance lethality in XPA-deficient cells after UV irradiation [Okano S, Kanno S, Nakajima S, Yasui A (2000) Cellular responses and repair of single-strand breaks introduced by UV damage endonuclease in mammalian cells. The Journal of biological chemistry 275 (42):32635-32641]. Tumor cells with deficiency of the DDR pathway have been suggested to be hypersensitive to PARP inhibitors, with the DNA repair biomarker CHEK1 shown to be overexpressed in BRCA1-like versus non-BRCA1-like triple negative breast cancer [Rodriguez A A, Makris A, Wu M F, Rimawi M, Froehlich A, Dave B, Hilsenbeck S G, Chamness G C, Lewis M T, Dobrolecki L E et al: DNA repair signature is associated with anthracycline response in triple negative breast cancer patients. Breast cancer research and treatment 2010, 123(1):189-196]. This is consistent with our finding that increased CHEK2 transcription is associated with olaparib sensitivity.

Our 7-gene transcript algorithm suggests that 8-21% of patients with primary breast cancers may respond to olaparib and that the responsive tumors are enriched in basal-like breast cancers. We present a signature that can be tested in planned translational analyses of ongoing clinical trials of PARP inhibitors and that can be used to determine whether clinical trials are properly sized to detect a response of the magnitude predicted by this signature.

Drug Response Data for Breast Cancer Cell Lines.

For measurement of sensitivity to KU0058948 (olaparib; KuDOS Pharmaceuticals/AstraZeneca), exponentially growing cells were seeded in six-well plates at a concentration of 5,000 cells per well. Cells were exposed continuously to the inhibitor, and medium and inhibitor were replaced every four days. After 15 days, cells were fixed and stained with sulphorhodamine-B (Sigma, St. Louis, USA) and a colorimetric assay performed as described previously [8]. Surviving fractions (SFs) were calculated and drug sensitivity curves determined with the Four Parameter Logistic Regression model as previously described [Farmer H, McCabe N, Lord C J, Tutt A N, Johnson D A, Richardson T B, Santarosa M, Dillon K J, Hickson I, Knights C et al: Targeting the DNA repair defect in BRCA mutant cells as a therapeutic strategy. Nature 2005, 434(7035):917-921].\

Molecular Data of Breast Cancer Cell Lines.

For copy number, DNA extracted from cell lines was labeled and hybridized to the Affymetrix Genome-Wide Human SNP Array 6.0 for DNA copy number. Data were segmented using the circular binary segmentation (CBS) algorithm from the Bioconductor package DNAcopy [73], followed by summarization at gene level with the R package CNTools. Human genome build 36 was used for processing and annotating. The segmented data are available on the Cancer Genomics Browser at UCSC under Stand Up To Cancer (https://genome-cancer.ucsc.edu/proj/site/hgHeatmap/). Gene expression data for the cell lines were derived from Affymetrix GeneChip Human Genome U133A and Affymetrix GeneChip Human Exon 1.0 ST arrays. U133A data was preprocessed with RMA in R, but with use of two distinct annotation files: standard annotation by Affymetrix followed by selection of the maximal varying probe set per gene, and a custom annotation to gene level [74]. The U133A expression data are available at http://cancer.lbl.gov/breastcancer/data.php. For the exon array, an improved mapping of the probes to human genome build 36.1 obtained by TCGA was used [60]. The raw data are available in ArrayExpress with accession number E-MTAB-181; processed data not shown. Whole transcriptome shotgun sequencing (RNA-seq) was completed on breast cancer cell lines and expression analysis was performed with the ALEXA-seq software package as previously described [75]. The processed log-transformed RNA-seq data for 20/22 cell lines is not shown. The Illumina Infinium Human Methylation27 BeadChip Kit was used for the genome-wide detection of the degree of methylation at 27,578 CpG loci, spanning 14,495 genes, with genome build 36 for annotation [98]. Reverse protein lysate array (RPPA) is an antibody-based method to quantitatively measure protein abundance [76] and was used for the measurement of 146 (phospho)proteins. Mutation data was extracted from COSMIC v53, the catalogue of somatic mutations in cancer [77]. Because contradictory PTEN mutation patterns have been reported in multiple studies and the COSMIC database, possibly due to cross-contamination and misidentification of cell lines, we used the re-sequencing results for the PTEN transcript obtained by Weigelt and colleagues [87] and independently confirmed in our lab (ICR). Due to the importance of post-translational modifications for PTEN function, we also used the PTEN protein and PTEN transcript levels assessed by western blotting [87]. We refer to [88] for a detailed description of the preprocessing of all molecular data sets.

Molecular Data of Tumor Samples.

U133A, U133B and U133 plus 2 expression data for 8 tumor sets (with Gene Expression Omnibus IDs GSE2034, GSE20271, GSE23988, GSE4922, GSE25066, GSE7390, GSE11121, GSE5460 [101]) were preprocessed with RMA in R with use of Affymetrix's standard annotation. Custom Agilent 244K expression data at gene level was available for 536 breast invasive carcinoma samples collected by TCGA (The Cancer Genome Atlas) as of Jan. 13, 2012 [71]. Missing values in this data set were imputed with KNNimputer in R [78]. Seven control genes previously obtained from breast tumor samples were used to correct for different tumor size, hormone receptor status and cell number between samples (ABI2, CXXC1, E2F4, GGA1, IPO8, RPL24, RPS10). The expression of the 7 signature genes was normalized to the geometric mean of all probe sets of the seven control genes [99]. The expression data sets were subsequently median normalized per gene across all samples. Before normalization to the control genes, the complete TCGA data set was quantile normalized per sample to a target distribution obtained from the U133A cell line data due to the difference in platform, thereby using functions ‘normalize.quantiles.determine.target’ and ‘normalize.quantiles.use.target’ from the R package affyPLM.

The TCGA tumor samples were subtyped with PAM50, a 50-gene set introduced for standardizing the categorical classification of breast cancer subtype into luminal A, luminal B, basal-like, HER2-enriched and normal-like [79]. The normal-like samples were excluded from the association study of subtype with response prediction to olaparib. For GSE25066, the subtypes assigned by Hatzis and colleagues were used [95].

Biomarker Selection and Model Building.

For biomarker selection, logistic regression (LR) with forward feature selection (5-fold CV) was opted for and applied to each DNA repair pathway separately. With forward feature selection, genes that result in the best data fit are consecutively added to the LR model. The difference in fit value when incorporating an additional gene is modeled with a chi-square distribution. When the gain in data fit is not significantly different from zero, no genes are further added to the LR model as not significantly improving the discriminatory power. LR model building was repeated 100 times to determine the most important markers selected in over half of the iterations. These markers were further reduced to those selected with consistent pattern of sensitivity for all 3 platforms (U133A with standard and custom annotation, exon array and RNA-seq) and for which the sensitivity pattern was independent of statistical measure (mean for fold-change vs. median for the weighted voting algorithm).

Before combining the resulting markers into a predictor, these markers were normalized to the geometric mean of the seven control genes described above, which were stable in the 22 cell lines. A predictor was subsequently obtained with use of the weighted voting algorithm [Moulder S, Yan K, Huang F, Hess K R, Liedtke C, Lin F, Hatzis C, Hortobagyi G N, Symmans W F, Pusztai L: Development of candidate genomic markers to select breast cancer patients for dasatinib therapy. Molecular cancer therapeutics 2010, 9(5):1120-1127]. For each gene g, the median μ and standard deviation σ of its median-normalized expression levels were calculated for the class of sensitive and resistant cell lines separately. The weight w_{g} and decision boundary b_{g} for gene g follows from

w_{g}=[μ₁(g)−μ₂(g)]/[σ₁(g)−σ₂(g)],

b_{g}=[μ₁(g)+μ₂(g)]/2.

For the calculation of predicted probability of response to olaparib for a new set of tumor samples, the expression data at logarithmic scale are median normalized for each gene g across all samples (X_{g}). The assignment of a new sample to the class of responders or non-responders follows from the sum of weighted votes across the set of biomarkers. For each individual biomarker g, the weighted vote V_{g} for a sample is calculated by subtracting the boundary value b_{g} from the gene expression value X_{g}, followed by multiplication of this difference with the biomarker weight w_{g} derived from the cell line data. After calculation of the weighted vote for all biomarkers, these votes are summed and compared to a threshold value obtained from the training data to determine the class the sample is assigned to. The absolute value of the difference between vote and threshold is an indication for the confidence of the class prediction.
X_{g}=median-normalized log expression level of gene g in a new sample

Weighted vote for gene g: V_{g}=w_{g}[X_{g}−b_{g}]

Total vote: S=ΣV_{g}

To obtain an optimal threshold value for dichotomization of vote S, the 7-gene predictor was applied to the U133A expression data (standard annotation) of the 22 cell lines and threshold 0.0372 was selected, corresponding to the largest accuracy for cell line response prediction.

Before validation of the 7-gene predictor on the TCGA Agilent data set, the threshold of 0.0372 was updated for Agilent because this platform was not used during signature development. An updated threshold of 0.174 was obtained by requiring the same prevalence for a set of 80 I-SPY1 tumor samples with both Affymetrix and Agilent data. Eighty-three samples in GSE25066 (Affymetrix U133A) were from the I-SPY 1 trial. For 80/83 samples, expression was additionally obtained with the Agilent 44K platform G4112 (GSE22226). Affymetrix U133A data of the I-SPY 1 samples were preprocessed in R with use of Affymetrix's standard annotation. Applying the 7-gene signature to these samples resulted in a prevalence of predicted response of 12%. We subsequently applied the 7-gene signature to the 80 I-SPY 1 samples with Agilent expression after quantile normalization, normalization with respect to the 7 internal genes, and median centering (similar as for TCGA described above). A prevalence of 12% was obtained with use of threshold 0.174. Predicted response of the 80 I-SPY 1 samples with expression data obtained with Affymetrix vs. Agilent were significantly correlated (Pearson correlation coefficient=0.278, p-value=0.012).

Statistical Analyses.

For the cell line panel, the Wilcoxon rank sum test was used to test the association of drug response with individual markers. Fold-change for each marker was calculated as the ratio of average marker expression in the sensitive with respect to the resistant cell lines, based on raw expression data [100]. Drug response was also associated with subtype, triple negativity and mutation status with use of the Fisher's exact test in R. Due to the small sample size, a p-value <0.05 was deemed significant whilst a p-value <0.1 was considered a trend. For the tumor samples, the chi-square test was used for the association of breast cancer subtype with response prediction to olaparib. All analyses were performed in Matlab R2010b for Mac, unless otherwise indicated.

Matlab code used for signature development of
Seven-Biomarker Predictor Panel

```
      Function BiomarkerSelection_
5foldCVrandomization_forwardSelection
determines for a particular expression data set (dataset) and gene set
from literature or KEGG (geneset) the genes that are selected by the logistic
regression approach across all randomizations
(SelectedGenes), with number of occurrences (nbOccurrences).
function [SelectedGenes nbOccurrences TestAUC] =
BiomarkerSelection_5foldCVrandomization_forwardSelection(dataset,
geneset)
nbRandomizations=100;
nrFolds=5;
%%% Import drug response data (cell line x drug matrix)
%%% (see Table 9 for the drug response data)
s=importdata(′DrugResponse_DataFile.txt′,′\t′);
% Cell with cell line names
celllines_drug=s.textdata(2:end,1);
% Vector with drug response values
drugdata=s.data;
% Set threshold for response dichotomization
threshold=1;
%%% Import the expression data set (gene x cell line matrix)
%%% (see Materials and Methods for a description of the
%%% expression data sets)
switch dataset
    case ′U133standard′
      %%% U133A - standard Affymetrix annotation, with the maximal
      %%% varying probe set per gene
      s=importdata(′U133standard_DataFile.txt′,′\t′);
      ExprData_full=s.data;
    case ′U133custom′
      %%% U133A - custom annotation file (Dai et al,
  %%% Nucleic Acids Res 2005)
      s=importdata(′U133custom_DataFile.txt′,′\t′);
      ExprData_full=s.data;
    case ′exon′
      %%% Exon array
      s=importdata(′ExonArray_DataFile.txt′,′\t′);
      ExprData_full=s.data;
    case ′RNAseq′
      %%% RNA-seq (log2-transformation required)
      s=importdata(′RNAseq_DataFile.txt′,′\t′);
      ExprData_full=log2(s.data+1);
end
Genes=s.textdata(2:end,1);
Celllines=s.textdata(1,2:end);
% Selection of cell lines with both expression and drug response data
[Celllines i_drug i_expr]=intersect(celllines_drug,Celllines);
ExprData_full=ExprData_full(:,i_expr);
drugdata=drugdata(i_drug);
% Binary outcome vector with 0 for cell lines with drug response >=
% threshold, and 1 for cell lines with drug response < threshold
response=zeros(1,length(drugdata));
response(drugdata<threshold)=1;
%%% Import prior set of DNA repair associated genes from literature
%%% (Wang et al, Am J Cancer Res, 2011) or from the KEGG database
%%% (see Table 15 for the list of genes)
switch geneset
    case ′Literature_HR′
      PriorGenes={′BRCA1′,′BRCA2′,′PTEN′,′USP11′,′PALB2′,...
        ′TP53BP1′,′RAD51′,′FANCD2′,′SHFM1′,′ATRX′,′RPA1′};
    case ′Literature_BER′
      PriorGenes={′PARP1′,′PARP2′,′JTB′};
    case ′Literature_NHEJ′
      PriorGenes={′PRKDC′,′XRCC5′,′XRCC6′};
    case ′Literature_NER′
      PriorGenes={′ERCC4′,′ERCC1′,′XPA′};
    case ′Literature_DDR′
      PriorGenes={′ATM′,′ATR′,′CHEK1′,′CHEK2′,′MRE11A′,′NBN′,...
′H2AFX′,′TP53′,′MAPKAPK2′};
    case ′KEGG_BER′
      PriorGenes=importdata(′KEGG_GeneList_BER.txt′);
    case ′KEGG_NER′
      PriorGenes=importdata(′KEGG_GeneList_NER.txt′);
    case ′KEGG_MMR′
      PriorGenes=importdata(′KEGG_GeneList_MMR.txt′);
    case ′KEGG_HR′
      PriorGenes=importdata(′KEGG_GeneList_HR.txt′);
    case ′KEGG_NHEJ′
      PriorGenes=importdata(′KEGG_GeneList_NHEJ.txt′);
end
% Reduction of the expression data set to the prior gene list
[GeneSet, ~, i_expr]=intersect(PriorGenes,Genes);
ExprData=ExprData_full(i_expr,:);
%%% Randomization approach with logistic regression and forward feature
%%% selection
% Selection of positive and negative cell lines
positives=find(response==1);
negatives=find(response==0);
% Generation of structures for the randomization results
b1Coeffs=cell(nrFolds,nbRandomizations);
pvalues=cell(nrFolds,nbRandomizations);
geneSets=cell(nrFolds,nbRandomizations); TestAUC=[ ]; AllGenes=[ ];
% Randomization outer loop
for i=1:nbRandomizations,
    % Randomized split of the cell lines into 5 folds,
    % stratified to outcome
    indicesPositives=nfCV(length(positives),nrFolds);
    indicesNegatives=nfCV(length(negatives),nrFolds);
    yfitTestAllGenes=ones(size(ExprData,2),1)*(−1);
    % 5-fold cross validation inner loop
    for fold=1:nrFolds
      % Training (4/5 folds) and test (1/5 folds) data generation
      testIndPos=find(indicesPositives==fold);
      testIndNeg=find(indicesNegatives==fold);
      trainIndPos=find(indicesPositives~=fold);
      trainIndNeg=find(indicesNegatives~=fold);
      Test=[positives(testIndPos) negatives(testIndNeg)];
      Train=[positives(trainIndPos) negatives(trainIndNeg)];
      GeneDataTrain=ExprData(:,Train);
      GeneDataTest=ExprData(:,Test);
      % Use sequential forward feature selection to rank genes
      % according to their contribution to the logistic regression
  % model
      [fs,history] =sequentialfs(@fitter,GeneDataTrain′,
[ones(1,length(trainIndPos)) zeros(1,length(trainIndNeg))]′,
′cv′,′none′,′nfeatures′,size(ExprData,1),′nullmodel′,true);
      % Set of deviance values for all models
      dev=history.Crit;
      % Deviance improvement for each step
      deltadev=−diff(dev);
      % Under the null hypothesis 2*deviance follows a
  % chi-square distribution
      maxdev = chi2inv(.95,1)/2;
      % Number of genes that significantly improved the model
  % when added
      nbfeatures = find(deltadev>maxdev,1, ′last′);
      if isempty(nbfeatures)
        nbfeatures = 0;
        in=false(1,size(ExprData,1));
      else
        in=logical(history.In(nbfeatures+1,:));
      end
      % Retrain the model with the selected markers and
  % validate on the left out test cell lines
      [b1 dev1 stat1] = glmfit(GeneDataTrain(in,:)′,
      [ones(1,length(trainIndPos))
zeros(1,length(trainIndNeg))]′, ′binomial′);
      geneSets{fold,i}=GeneSet(in);
      AllGenes=[AllGenes GeneSet(in)];
      b1Coeffs{fold,i}=b1;
      pvalues{fold,i}=stat1.p;
      yfitTestAllGenes(Test)=glmval(b1,GeneDataTest(in,:)′,′logit′);
    end
    % Calculation of performance and area under the receiver operating
    % characteristics curve for the prediction of the true labels
    % across the 5 cross validation iterations
    AREA=ROC2(yfitTestAllGenes,response);
    TestAUC=[TestAUC AREA];
  end
  % Calculation of the number of occurrences (out of 5×100=500
  % iterations) per gene in the selected gene set
  SelectedGenes=unique(AllGenes); nbOccurrences=[ ];
  for k=1:length(SelectedGenes),
    nbOccurrences=[nbOccurrences length
    (strmatch(SelectedGenes{k},AllGenes))];
  end
```

Function Validation validates the 7-gene signature derived from a 22-breast cancer cell line panel on an external gene x sample matrix. This function outputs the number of samples predicted to respond to olaparib according to the 7-gene signature (NumberPredictedResponders) and the corresponding percentage of samples predicted to respond (PercentagePredictedResponders).

When subtype information for the input samples is available, drug response prediction is associated with subtype. FrequencyTable_subtype contains per subtype the number of predicted non-responders and responders. When pathologic complete response for the input samples is available, drug response prediction is associated with pCR. FrequencyTable_pCR contains the number of predicted non-responders and responders for RD and pCR.

```
function [NumberPredictedResponders PercentagePredictedResponders
FrequencyTable_subtype FrequencyTable_pCR] =
Validation(Validation_Dataset)
%%% 7-gene signature
% Gene symbols and corresponding Affymetrix probes
GENES={′BRCA1′,′CHEK2′,′MAPKAPK2′,′MRE11A′,′NBN′,′TDG′,′XPA′};
PROBES={′204531_s_at′,′210416_s_at′,′201461_s_at′,′205395_s_at′,...
′202906_s_at′,′203743_s_at′,′205672_at′};
% Weights, boundaries and threshold of the 7-gene signature, obtained
% with the weighted voting algorithm (see Materials and
% Methods)
Weights=[−0.5320 0.5806 0.0713 −0.1396 −0.1976 −0.3937 −0.2335];
Boundaries=[−0.0153 −0.006 0.0031 −0.0044 0.0014 −0.0165 −0.0126];
THRESHOLD=0.0372;
%%% Import external tumor data set (gene x sample matrix)
s=importdata(Validation_Dataset);
TumorSamples=s.textdata(1,2:end);
ExprData=s.data;
GeneNames=s.textdata(2:end,1);
%%% Normalization of tumor data set with respect to set of 7 internal
%%% genes
% 7 internal normalization genes derived from tumor samples
GENES_NORM={′RPL24′,′ABI2′,′GGA1′,′E2F4′,′IPO8′,′CXXC1′,′RPS10′};
% Selection of expression data from the input tumor data set for the 7
% internal genes
% NOTE: Selection of corresponding probes is required when the input
% data is at probe level instead of gene level
indices_norm=[ ];
for i=1:length(GENES_NORM),
  indices_norm=[indices_norm;
  strmatch(GENES_NORM{i},GeneNames,′exact′)];
end
ExprData_norm=ExprData(indices_norm,:);
%%% Selection of expression data from the input tumor data set for the
%%% 7 signature genes
% NOTE: Selection of corresponding probes is required when the input
% data is at probe level instead of gene level
indices signature=[ ];
for i=1:length(GENES),
  indices_signature=[indices_signature
  strmatch(GENES{i},GeneNames,′exact′)];
end
ExprData_signature=ExprData(indices_ISPY1,:);
%%% Normalization of the expression data for the 7 signature genes to
%%% the geometric mean of the expression data for the 7 internal
%%% normalization genes, followed by median centering of the resulting
%%% data matrix
DATA=ExprData_signature./repmat(geomean(ExprData_norm,1),length
(indices_signature),1);
DATA=DATA-repmat(median(DATA,2),1,size(DATA,2));
%%% Testing of weighted voting algorithm
VotePos=zeros(1,size(DATA,2)); VoteNeg=zeros(1,size(DATA,2));
DistancePos=zeros(1,size(DATA,2)); DistanceNeg=zeros(1,size(DATA,2));
% Outer loop over all input samples
for i=1:size(DATA,2),
  % Inner loop over 7 signature genes
  WeightedVote=zeros(1,length(GENES));
  for j=1:size(DATA,1),
    WeightedVote(j)=Weights(j)*(DATA(j,i)-Boundaries(j));
  end
  indicesPos=WeightedVote>0;
  indicesNeg=WeightedVote<0;
  VotePos(i)=sum(WeightedVote(indicesPos));
  VoteNeg(i)=sum(WeightedVote(indicesNeg));
end
% Difference in total votes for the positive and negative class.
% The larger the difference, the more confident that the sample belongs
% to one class over the other class
DiffVote=VotePos-abs(VoteNeg);
%%% Comparison of predicted response to threshold 0.0372 obtained from
%%% the breast cancer cell line panel
NbPos=length(find(DiffVote>=THRESHOLD));
NbNeg=length(find(DiffVote<THRESHOLD));
NumberPredictedResponders=NbPos;
PercentagePredictedResponders=NbPos/length(DiffVote)*100;
%%% Association of predicted drug response with breast cancer subtype
%%% (when available)
% (sample x subtype matrix, with 1=lumA, 2=lumB, 3=basal,
% 4=ERBB2-amplified, 5=normal-like)
s=importdata(′Subtype_DataFile.txt′);
TumorSamples_subtype=s.textdata(2:end,1);
Subtypes=s.data(:,1);
% Select samples with both subtype and expression data
TumorSamplesCommon
i_expr i_subtype]=intersect(TumorSamples,TumorSamples_subtype);
Subtypes=Subtypes(i_subtype);
DiffVote_subtype=DiffVote(i_expr);
% Binarize predicted outcome based on the cell line-derived threshold
LabelPrediction=zeros(1,length(DiffVote subtype));
LabelPrediction(find(DiffVote_subtype>THRESHOLD))=1;
% Chi-square test for the association of subtype with predicted
% response (inclusion of lumA, lumB, basal, ERBB2-amplified and
% normal-like)
[tbl chi2 pvalue labels]=crosstab(Subtypes,LabelPrediction);
% Repetition of the association of subtype with predicted response with
% exclusion of normal-like samples
indicesNL=find(Subtypes==5);
LabelPrediction(indicesNL)=[ ];
Subtypes(indicesNL)=[ ];
[FrequencyTable_subtype chi2 pvalue
labels]=crosstab(Subtypes,LabelPrediction);
%%% Association of predicted drug response with pathologic complete
%%% response (when available)
% (sample x pCR matrix, with 1=pCR, 0=RD)
s=importdata(′pCR_DataFile.txt′);
TumorSamples_pCR=s.textdata(2:end,1);
pCR=s.data(:,1);
% Select samples with both subtype and expression data
[TumorSamplesCommon i_expr
i_pCR]=intersect(TumorSamples,TumorSamples_pCR);
pCR=pCR(i_pCR);
DiffVote_pCR=DiffVote(i_expr);
% Binarize predicted outcome based on the cell line-derived threshold
LabelPrediction=zeros(1,length(DiffVote_pCR));
LabelPrediction(find(DiffVote_pCR>THRESHOLD))=1;
% Chi-square test for the association of subtype with pCR
[FrequencyTable_pCR chi2 pvalue labels]=crosstab(pCR,LabelPrediction);
```

Function fitter builds a logistic regression model on data x with binary target vector y.

function dev=fitter(X,y)
[b,dev]=glmfit(X,y,′binomial′);

Function nfCV assigns N observations to K folds, and outputs the vector Ind indicating the fold to which each observation is assigned.

```
function Ind=nfCV(N,K)
  Ind = zeros(N,1);
  folds = ceil(K*(1:N)/N);
  Kperm = randperm(K);
  Nperm = randperm(N);
  Ind(Nperm)=Kperm(folds);
```

Function ROC2 calculates the area under the ROC curve (AREA), sensitivity (TPR_ROC), specificity (SPEC_ROC), accuracy (ACC_ROC), positive predictive value (PPV_ROC), negative predictive value (NPV_ROC), and false positive rate (FPR_ROC) at all possible thresholds (THRES_ROC), based on the continuous predictions (RESULT) and the true {0,1} labels (CLASS).

```
function [AREA,THRES_ROC,TPR_ROC,
SPEC_ROC,ACC_ROC,PPV_ROC,NPV_ROC,FPR_ROC] =
ROC2(RESULT,CLASS)
% NOTE: threshold is >, meaning that an element is considered to be
% positive when it is strictly larger than the threshold. The element
% is negative when <= threshold.
% Exclusion of NaN, Inf and −Inf elements
FI=find(isfinite(RESULT));
RESULT=(RESULT(FI));
CLASS=CLASS(FI);
FI=find(isfinite(CLASS));
RESULT=(RESULT(FI));
CLASS=CLASS(FI);
NRSAM=size(RESULT,1); % Number of samples
NN=sum(CLASS==0); % Number of true negative samples
NP=sum(CLASS==1); % Number of true positive samples
% Sort continuous predictions in ascending order, and corresponding
% rearrangement of the true labels
[RESULT_S,I]=sort(RESULT);
CLASS_S=CLASS(I);
TH=RESULT_S(NRSAM); % highest latent variable
% Initialisation (start with all cases as negative)
SAMNR=NRSAM; TP=0; FP=0; TN=NN; FN=NP; TPR=0; FPR=0; AREA=0;
THRES_ROC=[TH]; TPR_ROC=[TPR]; FPR_ROC=[FPR];
SPEC_ROC=[TN/(FP+TN)];
ACC_ROC=[(TP+TN/(NN+NP)]; PPV_ROC=[NaN];
NPV_ROC=[TN/(TN+FN)];
while ~isempty(TH)
  % indices of cases with a prediction equal to TH
  DELTA=CLASS_S(RESULT_S==TH);
  % number of negative samples, predicted as positive at threshold TH
  DFP=sum(DELTA==0);
  % number of positive samples, predicted as positive at threshold TH
  DTP=sum(DELTA==1);
  % TN = number of negative samples characterized as negative
  TN=TN−DFP;
  % AREA = area under the receiver characteristics curve
  AREA=AREA + DFP*TP + 0.5*DFP*DTP;
  % FP = number of negative samples characterized as positive
  FP=FP+DFP;
  % TP = number of positive samples characterized as positive
  TP=TP+DTP;
  % FN = number of positive samples characterized as negative
  FN=FN−DTP;
  TPR=TP/(TP+FN); % TPR = true positive rate
  FPR=FP/(FP+TN); % FPR = false positive rate
  % Selection of next threshold
  SAMNR=find(RESULT_S<TH,1,′last′);
  TH=RESULT_S(SAMNR);
  TPR_ROC=[TPR_ROC; TPR];
  FPR_ROC=[FPR_ROC; FPR];
  THRES_ROC=[THRES_ROC; TH];
  SPEC_ROC=[SPEC_ROC; TN/ (FP+TN)];
  ACC_ROC=[ACC_ROC; (TP+TN)/(NN+NP)];
  if (TP+FP) ==0
    PPV_ROC=[PPV_ROC; NaN];
  else
    PPV_ROC=[PPV_ROC; TP/(TP+FP)];
  end
  if (TN+FN) ==0
    NPV_ROC=[NPV_ROC; NaN];
  else
    NPV_ROC=[NPV_ROC; TN/(TN+FN)];
    end
end
THRES_ROC=ROC; −1];
AREA=AREA/ (NN*NP);
TPR_ROC=TPR_ROC*100;
FPR_ROC=FPR_ROC*100;
SPEC_ROC=SPEC_ROC*100;
ACC_ROC=ACC_ROC*100;
PPV_ROC=PPV_ROC*100;
NPV_ROC=NPV_ROC*100;
```

### REFERENCES CITED

1. Rich T, Allen R L, Wyllie A H: Defying death after DNA damage. Nature 2000, 407(6805):777-783.
2. Wang X, Weaver D: The ups and downs of DNA repair biomarkers for PARP inhibitor therapies. Am J Cancer Res 2011, 1(3):301-327.
3. Sancar A, Lindsey-Boltz L A, Unsal-Kacmaz K, Linn S: Molecular mechanisms of mammalian DNA repair and the DNA damage checkpoints. Annual review of biochemistry 2004, 73:39-85.
4. Ciccia A, Elledge S J: The DNA damage response: making it safe to play with knives. Molecular cell 2010, 40(2):179-204.
5. Iglehart J D, Silver D P: Synthetic lethality—a new direction in cancer-drug development. The New England journal of medicine 2009, 361(2):189-191.
6. Bryant H E, Schultz N, Thomas H D, Parker K M, Flower D, Lopez E, Kyle S, Meuth M, Curtin N J, Helleday T: Specific killing of BRCA2-deficient tumours with inhibitors of poly(ADP-ribose) polymerase. Nature 2005, 434(7035):913-917.
7. Farmer H, McCabe N, Lord C J, Tutt A N, Johnson D A, Richardson T B, Santarosa M, Dillon K J, Hickson I, Knights C et al: Targeting the DNA repair defect in BRCA mutant cells as a therapeutic strategy. Nature 2005, 434(7035):917-921.
8. Edwards S L, Brough R, Lord C J, Natrajan R, Vatcheva R, Levine D A, Boyd J, Reis-Filho J S, Ashworth A: Resistance to therapy caused by intragenic deletion in BRCA2. Nature 2008, 451(7182):1111-1115.
9. Gudmundsdottir K, Ashworth A: The roles of BRCA1 and BRCA2 and associated proteins in the maintenance of genomic stability. Oncogene 2006, 25(43):5864-5874.
10. Tutt A, Ashworth A: The relationship between the roles of BRCA genes in DNA repair and cancer predisposition. Trends in molecular medicine 2002, 8(12):571-576.
11. Narod S A, Foulkes W D: BRCA1 and BRCA2: 1994 and beyond. Nature reviews Cancer 2004, 4(9):665-676.
12. Rouleau M, Patel A, Hendzel M J, Kaufmann S H, Poirier G G: PARP inhibition: PARP1 and beyond. Nature reviews Cancer 2010, 10(4):293-301.
13. Liang H, Tan A: PARP inhibitors. Curr Breast Cancer Rep 2011, 3:44-54.
14. Underhill C, Toulmonde M, Bonnefoi H: A review of PARP inhibitors: from bench to bedside. Annals of oncology: official journal of the European Society for Medical Oncology/ESMO 2011, 22(2):268-279.
15. Guha M: PARP inhibitors stumble in breast cancer. Nature biotechnology 2011, 29(5):373-374.
16. Vinayak S, Ford J: PARP inhibitors for the treatment and prevention of breast cancer. Curr Breast Cancer Rep 2010, 2:190-197.
17. Rouleau M, Patel A, Hendzel M J, Kaufmann S H, Poirier G G: PARP inhibition: PARP1 and beyond. Nature reviews Cancer 2010, 10(4):293-301Plummer R: Poly(ADP-ribose) polymerase inhibition: a new direction for BRCA and triple-negative breast cancer? Breast cancer research: BCR 2011, 13(4):218.
18. Turner N, Tutt A, Ashworth A: Hallmarks of ‘BRCAness’ in sporadic cancers. Nature reviews Cancer 2004, 4(10):814-819.
19. O'Shaughnessy J, Osborne C, Pippen J E, Yoffe M, Patt D, Rocha C, Koo I C, Sherman B M, Bradley C: Iniparib plus chemotherapy in metastatic triple-negative breast cancer. The New England journal of medicine 2011, 364(3):205-214.
20. O'Shaughnessy J, Schwartzberg L, Danso M, Rugo H, Miller K, Yardley D, Carlson R, Finn R, Charpentier E, Freese M et al: A randomized phase III study of iniparib (BSI-201) in combination with gemcitabine/carboplatin (G/C) in metastatic triple-negative breast cancer (TNBC). J Clin Oncol 2011, 29:suppl; abstr 1007.
21. Turner N C, Ashworth A: Biomarkers of PARP inhibitor sensitivity. Breast cancer research and treatment 2011, 127(1):283-286.
22. Fong P C, Boss D S, Yap T A, Tutt A, Wu P, Mergui-Roelvink M, Mortimer P, Swaisland H, Lau A, O'Connor M J et al: Inhibition of poly(ADP-ribose) polymerase in tumors from BRCA mutation carriers. The New England journal of medicine 2009, 361(2):123-134.
23. Negrini S, Gorgoulis V G, Halazonetis T D: Genomic instability—an evolving hallmark of cancer. Nature reviews Molecular cell biology 2010, 11(3):220-228.
24. Mendes-Pereira A M, Martin S A, Brough R, McCarthy A, Taylor J R, Kim J S, Waldman T, Lord C J, Ashworth A: Synthetic lethal targeting of PTEN mutant cells with PARP inhibitors. EMBO molecular medicine 2009, 1(6-7):315-322.
25. McEllin B, Camacho C V, Mukherjee B, Hahm B, Tomimatsu N, Bachoo R M, Burma S: PTEN loss compromises homologous recombination repair in astrocytes: implications for glioblastoma therapy with temozolomide or poly(ADP-ribose) polymerase inhibitors. Cancer research 2010, 70(13):5457-5464.
26. Dedes K J, Wetterskog D, Mendes-Pereira A M, Natrajan R, Lambros M B, Geyer F C, Vatcheva R, Savage K, Mackay A, Lord C J et al: PTEN deficiency in endometrioid endometrial adenocarcinomas predicts sensitivity to PARP inhibitors. Science translational medicine 2010, 2(53):53ra75.
27. Williamson C T, Muzik H, Turhan A G, Zamo A, O'Connor M J, Bebb D G, Lees-Miller S P: ATM deficiency sensitizes mantle cell lymphoma cells to poly(ADP-ribose) polymerase-1 inhibitors. Molecular cancer therapeutics 2010, 9(2):347-357.
28. Holstege H, Horlings H M, Velds A, Langerod A, Borresen-Dale A L, van de Vijver M J, Nederlof P M, Jonkers J: BRCA1-mutated and basal-like breast cancers have similar aCGH profiles and a high incidence of protein truncating T P53 mutations. BMC cancer 2010, 10:654.
29. Goncalves A, Finetti P, Sabatier R, Gilabert M, Adelaide J, Borg J P, Chaffanet M, Viens P, Birnbaum D, Bertucci F: Poly(ADP-ribose) polymerase-1 mRNA expression in human breast cancer: a meta-analysis. Breast cancer research and treatment 2011, 127(1):273-281.
30. Domagala P, Huzarski T, Lubinski J, Gugala K, Domagala W: Immunophenotypic predictive profiling of BRCA1-associated breast cancer. Virchows Archiv: an international journal of pathology 2011, 458(1):55-64.
31. Cotter M, Pierce A, McGowan P, Madden S, Flanagan L, Quinn C, Evoy D, Crown J, McDermott E, Duffy M: PARP1 in triple-negative breast cancer: expression and therapeutic potential. J Clin Oncol 2011, 29(15_suppl):1061.
32. Zaremba T, Ketzer P, Cole M, Coulthard S, Plummer E R, Curtin N J: Poly(ADP-ribose) polymerase-1 polymorphisms, expression and activity in selected human tumour cell lines. British journal of cancer 2009, 101(2):256-262.
33. De Soto J, Mullins R: The use of PARP inhibitors as single agents and as chemosensitizers in sporadic pancreatic cancer. J Clin Oncol 2011, 29(15_suppl):e13542.
34. LoRusso P, Ji J, Li J, Heilbrun L, Shapiro G, Sausville E, Boerner S, Smith D, Pilat M, Zhang J et al: Phase I study of the safety, pharmacokinetics (PK), and pharmacodynamics (PD) of the poly(ADP-ribose) polymerase (PARP) inhibitor veliparib (ABT-888; V) in combination with irinotecan (CPT-11; Ir) in patients (pts) with advanced solid tumors. J Clin Oncol 2011, 29(15_suppl):3000.
35. Lee J, Annunziata C, Minasian L, Zujewski J, Prindiville S, Kotz H, Squires J, Houston N, Ji J, Yu M et al: Phase I study of the PARP inhibitor olaparib (O) in combination with carboplatin (C) in BRCA1/2 mutation carriers with breast (Br) or ovarian (Ov) cancer (Ca). J Clin Oncol 2011, 29(15_suppl):2520.
36. McCabe N, Turner N C, Lord C J, Kluzek K, Bialkowska A, Swift S, Giavara S, O'Connor M J, Tutt A N, Zdzienicka M Z et al: Deficiency in the repair of DNA damage by homologous recombination and sensitivity to poly(ADP-ribose) polymerase inhibition. Cancer research 2006, 66(16):8109-8115.
37. Wiltshire T D, Lovejoy C A, Wang T, Xia F, O'Connor M J, Cortez D: Sensitivity to poly(ADP-ribose) polymerase (PARP) inhibition identifies ubiquitin-specific peptidase 11 (USP11) as a regulator of DNA double-strand break repair. The Journal of biological chemistry 2010, 285(19):14565-14571.
38. Rodriguez A A, Makris A, Wu M F, Rimawi M, Froehlich A, Dave B, Hilsenbeck S G, Chamness G C, Lewis M T, Dobrolecki L E et al: DNA repair signature is associated with anthracycline response in triple negative breast cancer patients. Breast cancer research and treatment 2010, 123(1):189-196.
39. Banuelos C A, Banath J P, Kim J Y, Aquino-Parsons C, Olive P L: gammaH2A X expression in tumors exposed to cisplatin and fractionated irradiation. Clinical cancer research: an official journal of the American Association for Cancer Research 2009, 15(10):3344-3353.
40. Bonner W M, Redon C E, Dickey J S, Nakamura A J, Sedelnikova O A, Solier S, Pommier Y: GammaH2A X and cancer. Nature reviews Cancer 2008, 8(12):957-967.
41. Mukhopadhyay A, Elattar A, Cerbinskaite A, Wilkinson S J, Drew Y, Kyle S, Los G, Hostomsky Z, Edmondson R J, Curtin N J: Development of a functional assay for homologous recombination status in primary cultures of epithelial ovarian tumor and correlation with sensitivity to poly(ADP-ribose) polymerase inhibitors. Clinical cancer research: an official journal of the American Association for Cancer Research 2010, 16(8):2344-2351.
42. Baldassarre G, Battista S, Belletti B, Thakur S, Pentimalli F, Trapasso F, Fedele M, Pierantoni G, Croce C M, Fusco A: Negative regulation of BRCA1 gene expression by HMGA1 proteins accounts for the reduced BRCA1 protein levels in sporadic breast carcinoma. Molecular and cellular biology 2003, 23(7):2225-2238.
43. Beger C, Pierce L N, Kruger M, Marcusson E G, Robbins J M, Welcsh P, Welch P J, Welte K, King M C, Barber J R et al: Identification of Id4 as a regulator of BRCA1 expression by using a ribozyme-library-based inverse genomics approach. Proceedings of the National Academy of Sciences of the United States of America 2001, 98(1):130-135.
44. Turner N C, Reis-Filho J S, Russell A M, Springall R J, Ryder K, Steele D, Savage K, Gillett C E, Schmitt F C, Ashworth A et al: BRCA1 dysfunction in sporadic basal-like breast cancer. Oncogene 2007, 26(14):2126-2132.
45. Lemee F, Bergoglio V, Fernandez-Vidal A, Machado-Silva A, Pillaire M J, Bieth A, Gentil C, Baker L, Martin A L, Leduc C et al: DNA polymerase theta up-regulation is associated with poor survival in breast cancer, perturbs DNA replication, and promotes genetic instability. Proceedings of the National Academy of Sciences of the United States of America 2010, 107(30):13390-13395.
46. Sourisseau T, Maniotis D, McCarthy A, Tang C, Lord C J, Ashworth A, Linardopoulos S: Aurora-A expressing tumour cells are deficient for homology-directed DNA double strand-break repair and sensitive to PARP inhibition. EMBO molecular medicine 2010, 2(4):130-142.
47. Esteller M, Silva J M, Dominguez G, Bonilla F, Matias-Guiu X, Lerma E, Bussaglia E, Prat J, Harkes I C, Repasky E A et al: Promoter hypermethylation and BRCA1 inactivation in sporadic breast and ovarian tumors. Journal of the National Cancer Institute 2000, 92(7):564-569.
48. Magdinier F, Dante R: Analysis of the DNA methylation patterns at the BRCA1 CpG island. Biochemica 2006, 3:13-15.
49. Catteau A, Harris W H, Xu C F, Solomon E: Methylation of the BRCA1 promoter region in sporadic breast and ovarian cancer: correlation with disease characteristics. Oncogene 1999, 18(11):1957-1965.
50. Olopade O I, Wei M: FANCF methylation contributes to chemoselectivity in ovarian cancer. Cancer cell 2003, 3(5):417-420.
51. Turner N C, Lord C J, Iorns E, Brough R, Swift S, Elliott R, Rayter S, Tutt A N, Ashworth A: A synthetic lethal siRNA screen identifying genes mediating sensitivity to a PARP inhibitor. The EMBO journal 2008, 27(9):1368-1377.
52. Barker A D, Sigman C C, Kelloff G J, Hylton N M, Berry D A, Esserman L J: I-SPY 2: an adaptive breast cancer trial design in the setting of neoadjuvant chemotherapy. Clinical pharmacology and therapeutics 2009, 86(1):97-100.
53. Esserman L, Perou C, Cheang M, DeMichele A, Carey L, van 't Veer L, Gray J, Petricoin E, Conway K, Berry D: Breast cancer molecular profiles and tumor response of neoadjuvant doxorubicin and paclitaxel: The I-SPY TRIAL (CALGB 150007/150012, ACRIN 6657). J Clin Oncol 2009, 27(18s):suppl; abstr LBA515.
54. Hylton N, Blume J, Gatsonis C, Gomez R, Bernreuter W, Pisano E, Rosen M, Marques H, Esserman L, Schnall M: MRI tumor volume for predicting response to neoadjuvant chemotherapy in locally advanced breast cancer: Findings from ACRIN 6657/CALGB 150007. J Clin Oncol 2009, 27(15s):suppl; abstr 529.
55. Lin C, Moore D, DeMichele A, Ollila D, Montgomery L, Liu M, Krontiras H, Gomez R, Esserman L: Detection of locally advanced breast cancer in the I-SPY TRIAL (CALGB 150007/150012, ACRIN 6657) in the interval between routine screening. J Clin Oncol 2009, 27(15s):suppl; abstr 1503.
56. Berry D A: Bayesian clinical trials. Nature reviews Drug discovery 2006, 5(1):27-36.
57. Sotiriou C, Pusztai L: Gene-expression signatures in breast cancer. The New England journal of medicine 2009, 360(8):790-800.
58. Neve R M, Chin K, Fridlyand J, Yeh J, Baehner F L, Fevr T, Clark L, Bayani N, Coppe J P, Tong F et al: A collection of breast cancer cell lines for the study of functionally distinct cancer subtypes. Cancer cell 2006, 10(6):515-527.
59. Saal L H, Gruvberger-Saal S K, Persson C, Lovgren K, Jumppanen M, Staaf J, Jonsson G, Pires M M, Maurer M, Holm K et al: Recurrent gross mutations of the PTEN tumor suppressor gene in breast cancers with deficient DSB repair. Nature genetics 2008, 40(1):102-107.
60. Integrated genomic analyses of ovarian carcinoma. Nature 2011, 474(7353):609-615.
61. Szabo C I, Worley T, Monteiro A N: Understanding germ-line mutations in BRCA1. Cancer biology & therapy 2004, 3(6):515-520.
62. Shattuck-Eidens D, McClure M, Simard J, Labrie F, Narod S, Couch F, Hoskins K, Weber B, Castilla L, Erdos M et al: A collaborative survey of 80 mutations in the BRCA1 breast and ovarian cancer susceptibility gene. Implications for presymptomatic testing and screening. JAMA: the journal of the American Medical Association 1995, 273(7):535-541.
63. Sakai W, Swisher E M, Karlan B Y, Agarwal M K, Higgins J, Friedman C, Villegas E, Jacquemont C, Farrugia D J, Couch F J et al: Secondary mutations as a mechanism of cisplatin resistance in BRCA2-mutated cancers. Nature 2008, 451(7182):1116-1120.
64. Kanehisa M, Goto S, Furumichi M, Tanabe M, Hirakawa M: KEGG for representation and analysis of molecular networks involving diseases and drugs. Nucleic acids research 2010, 38(Database issue):D355-360.
65. Williams G J, Lees-Miller S P, Tainer J A: Mre11-Rad50-Nbs1 conformations and the control of sensing, signaling, and effector responses at DNA double-strand breaks. DNA repair 2010, 9(12):1299-1306.
66. Vilar E, Bartnik C M, Stenzel S L, Raskin L, Ahn J, Moreno V, Mukherjee B, Iniesta M D, Morgan M A, Rennert G et al: MRE11 deficiency increases sensitivity to poly(ADP-ribose) polymerase inhibition in microsatellite unstable colorectal cancers. Cancer research 2011, 71(7):2632-2642.
67. Wen Q, Scorah J, Phear G, Rodgers G, Rodgers S, Meuth M: A mutant allele of MRE11 found in mismatch repair-deficient tumor cells suppresses the cellular response to DNA replication fork stress in a dominant negative manner. Molecular biology of the cell 2008, 19(4):1693-1705.
68. Mahaney B L, Meek K, Lees-Miller S P: Repair of ionizing radiation-induced DNA double-strand breaks by non-homologous end-joining. The Biochemical journal 2009, 417(3):639-650.
69. Loser D A, Shibata A, Shibata A K, Woodbine L J, Jeggo P A, Chalmers A J: Sensitization to radiation and alkylating agents by inhibitors of poly(ADP-ribose) polymerase is enhanced in cells deficient in DNA double-strand break repair. Molecular cancer therapeutics 2010, 9(6):1775-1787.
70. Moulder S, Yan K, Huang F, Hess K R, Liedtke C, Lin F, Hatzis C, Hortobagyi G N, Symmans W F, Pusztai L: Development of candidate genomic markers to select breast cancer patients for dasatinib therapy. Molecular cancer therapeutics 2010, 9(5):1120-1127.
71. The Cancer Genome Atlas Data Portal, available at http://tcga-data.nci.nih.gov/tcga/tcgaHome2.jsp
72. Van Rijsbergen C: Information retrieval: Butterworth; 1979.
73. Venkatraman E S, Olshen A B: A faster circular binary segmentation algorithm for the analysis of array CGH data. Bioinformatics 2007, 23(6):657-663.
74. Dai M, Wang P, Boyd A D, Kostov G, Athey B, Jones E G, Bunney W E, Myers R M, Speed T P, Akil H et al: Evolving gene/transcript definitions significantly alter the interpretation of GeneChip data. Nucleic acids research 2005, 33(20):e175.
75. Griffith M, Griffith O L, Mwenifumbo J, Goya R, Morrissy A S, Morin R D, Corbett R, Tang M J, Hou Y C, Pugh T J et al: Alternative expression analysis by RNA sequencing. Nat Methods 2010, 7(10):843-847.
76. Tibes R, Qiu Y, Lu Y, Hennessy B, Andreeff M, Mills G B, Kornblau S M: Reverse phase protein array: validation of a novel proteomic technology and utility for analysis of primary leukemia specimens and hematopoietic stem cells. Mol Cancer Ther 2006, 5(10):2512-2521.
77. Forbes S A, Bhamra G, Bamford S, Dawson E, Kok C, Clements J, Menzies A, Teague J W, Futreal P A, Stratton M R: The Catalogue of Somatic Mutations in Cancer (COSMIC). Curr Protoc Hum Genet. 2008, Chapter 10:Unit 10 11.
78. Troyanskaya O, Cantor M, Sherlock G, Brown P, Hastie T, Tibshirani R, Botstein D, Altman R B: Missing value estimation methods for DNA microarrays. Bioinformatics 2001, 17(6):520-525.
79. Parker J S, Mullins M, Cheang M C, Leung S, Voduc D, Vickery T, Davies S, Fauron C, He X, Hu Z et al: Supervised risk predictor of breast cancer based on intrinsic subtypes. J Clin Oncol 2009, 27(8):1160-1167.
80. Ashworth A, Lord C J, Reis-Filho J S (2011) Genetic interactions in cancer progression and treatment. Cell 145 (1):30-38. doi:10.1016/j.cell.2011.03.020
81. Loveday C, Turnbull C, Ramsay E, Hughes D, Ruark E, Frankum J R, Bowden G, Kalmyrzaev B, Warren-Perry M, Snape K, Adlard J W, Barwell J, Berg J, Brady A F, Brewer C, Brice G, Chapman C, Cook J, Davidson R, Donaldson A, Douglas F, Greenhalgh L, Henderson A, Izatt L, Kumar A, Lalloo F, Miedzybrodzka Z, Morrison P J, Paterson J, Porteous M, Rogers M T, Shanley S, Walker L, Eccles D, Evans D G, Renwick A, Seal S, Lord C J, Ashworth A, Reis-Filho J S, Antoniou A C, Rahman N (2011) Germline mutations in RAD51D confer susceptibility to ovarian cancer. Nature genetics 43 (9):879-882. doi:10.1038/ng.893
82. Buisson R, Dion-Cote A M, Coulombe Y, Launay H, Cai H, Stasiak A Z, Stasiak A, Xia B, Masson J Y (2010) Cooperation of breast cancer proteins PALB2 and piccolo BRCA2 in stimulating homologous recombination. Nature structural & molecular biology 17 (10):1247-1254. doi:10.1038/nsmb.1915
83. Caldecott K W (2007) Mammalian single-strand break repair: mechanisms and links with chromatin. DNA repair 6 (4):443-453. doi:10.1016/j.dnarep.2006.10.006
84. Tutt A, Robson M, Garber J E, Domchek S M, Audeh M W, Weitzel J N, Friedlander M, Arun B, Loman N, Schmutzler R K, Wardley A, Mitchell G, Earl H, Wickens M, Carmichael J (2010) Oral poly(ADP-ribose) polymerase inhibitor olaparib in patients with BRCA1 or BRCA2 mutations and advanced breast cancer: a proof-of-concept trial. Lancet 376 (9737):235-244. doi:10.1016/S0140-6736(10)60892-6
85. Dent R, Lindeman G, Clemons M, Wildiers H, Chan A, McCarthy N, Singer C, Lowe E, Kemsley K, Carmichael J (2010) Safety and efficacy of the oral PARP inhibitor olaparib (AZD2281) in combination with paclitaxel for the 1st or 2nd line treatment of patients with metastatic triple negative breast cancer: Results from the safety cohort of a Phase 1/2 multicentre trial. Proc Am Soc Clin Oncol 28 (suppl):abstr 1018
86. Gelmon K A, Tischkowitz M, Mackay H, Swenerton K, Robidoux A, Tonkin K, Hirte H, Huntsman D, Clemons M, Gilks B, Yerushalmi R, Macpherson E, Carmichael J, Oza A (2011) Olaparib in patients with recurrent high-grade serous or poorly differentiated ovarian carcinoma or triple-negative breast cancer: a phase 2, multicentre, open-label, non-randomised study. The lancet oncology 12 (9):852-861. doi:10.1016/S1470-2045(11)70214-5
87. Weigelt B, Warne P H, Downward J (2011) PIK3C A mutation, but not PTEN loss of function, determines the sensitivity of breast cancer cells to mTOR inhibitory drugs. Oncogene 30 (29):3222-3233. doi:10.1038/one.2011.42
88. Heiser L M, Sadanandam A, Kuo W L, Benz S C, Goldstein T C, Ng S, Gibb W J, Wang N J, Ziyad S, Tong F, Bayani N, Hu Z, Billig J I, Dueregger A, Lewis S, Jakkula L, Korkola J E, Durinck S, Pepin F, Guan Y, Purdom E, Neuvial P, Bengtsson H, Wood K W, Smith P G, Vassilev L T, Hennessy B T, Greshock J, Bachman K E, Hardwicke M A, Park J W, Marton L J, Wolf D M, Collisson E A, Neve R M, Mills G B, Speed T P, Feiler H S, Wooster R F, Haussler D, Stuart J M, Gray J W, Spellman P T (2012) Subtype and pathway specific responses to anticancer compounds in breast cancer. Proceedings of the National Academy of Sciences of the United States of America 109 (8):2724-2729. doi:10.1073/pnas.1018854108
89. McShane L M, Altman D G, Sauerbrei W, Taube S E, Gion M, Clark G M (2006) REporting recommendations for tumor MARKer prognostic studies (REMARK). Breast cancer research and treatment 100 (2):229-235. doi:10.1007/s10549-006-9242-8
90. Graeser M, McCarthy A, Lord C J, Savage K, Hills M, Salter J, On N, Parton M, Smith I E, Reis-Filho J S, Dowsett M, Ashworth A, Turner N C (2010) A marker of homologous recombination predicts pathologic complete response to neoadjuvant chemotherapy in primary breast cancer. Clinical cancer research: an official journal of the American Association for Cancer Research 16 (24):6159-6168. doi:10.1158/1078-0432.CCR-10-1027
91. CHEK2 Breast Cancer Case-Control Consortium (2004) CHEK2*1100delC and susceptibility to breast cancer: a collaborative analysis involving 10,860 breast cancer cases and 9,065 controls from 10 studies. American journal of human genetics 74 (6):1175-1182. doi:10.1086/421251
92. Fletcher O, Johnson N, Dos Santos Silva I, Kilpivaara O, Aittomaki K, Blomqvist C, Nevanlinna H, Wasielewski M, Meijers-Heijerboer H, Broeks A, Schmidt M K, Van't Veer L J, Bremer M, Dork T, Chekmariova E V, Sokolenko A P, Imyanitov E N, Hamann U, Rashid M U, Brauch H, Justenhoven C, Ashworth A, Peto J (2009) Family history, genetic testing, and clinical risk prediction: pooled analysis of CHEK2 1100delC in 1,828 bilateral breast cancers and 7,030 controls. Cancer epidemiology, biomarkers & prevention: a publication of the American Association for Cancer Research, cosponsored by the American Society of Preventive Oncology 18 (1):230-234. doi:10.1158/1055-995.EPI-08-0416
93. Reinhardt H C, Aslanian A S, Lees J A, Yaffe M B (2007) p53-deficient cells rely on ATM- and ATR-mediated checkpoint signaling through the p38MAPK/M K2 pathway for survival after DNA damage. Cancer cell 11 (2):175-189. doi:10.1016/j.ccr.2006.11.024
94. Reinhardt H C, Hasskamp P, Schmedding I, Morandell S, van Vugt M A, Wang X, Linding R, Ong S E, Weaver D, Carr S A, Yaffe M B (2010) DNA damage activates a spatially distinct late cytoplasmic cell-cycle checkpoint network controlled by M K2-mediated RNA stabilization. Molecular cell 40 (1):34-49. doi:10.1016/j.molcel.2010.09.018
95. Hatzis C, Pusztai L, Valero V, Booser D J, Esserman L, Lluch A, Vidaurre T, Holmes F, Souchon E, Wang H, Martin M, Cotrina J, Gomez H, Hubbard R, Chacon J I, Ferrer-Lozano J, Dyer R, Buxton M, Gong Y, Wu Y, Ibrahim N, Andreopoulou E, Ueno N T, Hunt K, Yang W, Nazario A, DeMichele A, O'Shaughnessy J, Hortobagyi G N, Symmans W F (2011) A genomic predictor of response and survival following taxane-anthracycline chemotherapy for invasive breast cancer. JAMA: the journal of the American Medical Association 305 (18):1873-1881. doi:10.1001/jama.2011.593
96. Koberle B, Masters J R, Hartley J A, Wood R D (1999) Defective repair of cisplatin-induced DNA damage caused by reduced XPA protein in testicular germ cell tumours. Current biology: CB 9 (5):273-276
97. Okano S, Kanno S, Nakajima S, Yasui A (2000) Cellular responses and repair of single-strand breaks introduced by UV damage endonuclease in mammalian cells. The Journal of biological chemistry 275 (42):32635-32641. doi:10.1074/jbc.M004085200
98. Fackler M J, Umbricht C, Williams D, Argani P, Cruz L A, Merino V F, Teo W W, Zhang Z, Huang P, Visvanathan K et al: Genome-Wide Methylation Analysis Identifies Genes Specific to Breast Cancer Hormone Receptor Status and Risk of Recurrence. Cancer research 2011.
99. Vandesompele J, De Preter K, Pattyn F, Poppe B, Van Roy N, De Paepe A, Speleman F: Accurate normalization of real-time quantitative R T-PCR data by geometric averaging of multiple internal control genes. Genome biology 2002, 3(7):RESEARCH0034.
100. Tusher V G, Tibshirani R, Chu G: Significance analysis of microarrays applied to the ionizing radiation response. Proceedings of the National Academy of Sciences of the United States of America 2001, 98(9):5116-5121.
101. Gene Expression Omnibus, available at NCBI GEO website.

The above description, tables and examples are provided to illustrate the invention but not to limit its scope. Other variants of the invention will be readily apparent to one of ordinary skill in the art and are encompassed by the appended claims. All publications, databases, and patents cited herein are hereby incorporated by reference for all purposes.

TABLE 1

| | | | | | | Decision |
|---|---|---|---|---|---|---|
| Gene | Entrez | Main gene | Marker | Affymetrix | Weight | boundary |
| symbol | gene ID | function | pattern | U133A probe | w_{g} | b_{g} |
| BRCA1 | 672 | DSB repair via | Resistant | 204531_s_at | −0.252 | 0.0451 |
| BRCA2 | 675 | RAD51-mediated HR | Sensitive | 214727_at | 0.0817 | −0.0191 |
| CHEK1 | 1111 | Kinases involved in | Sensitive | 205393_s_at | 0.0674 | 0.0277 |
| CHEK2 | 11200 | two major DDR | Sensitive | 210416_s_at | 0.4788 | 0.0119 |
| | | pathways ATR-Chk1 | | | | |
| | | and ATM-Chk2 | | | | |
| MRE11A | 4361 | MRN complex for DSB | Resistant | 205395_s_at | −0.2372 | −0.0331 |
| | | recognition | | | | |
| γH2AX | 3014 | γH2AX foci formed | Resistant | 205436_s_at | −0.3483 | −0.0397 |
| | | with~every DSB and | | | | |
| | | involved in DSB repair | | | | |
| | | by HR and NHEJ | | | | |
| TDG | 6996 | BER pathway | Resistant | 203743_s_at | −0.8039 | −0.1046 |
| XRCC5 | 7520 | Forms Ku70/Ku80 | Resistant | 208643_s_at | −0.3715 | 0.0181 |
| (Ku80) | | heterodimer that | | | | |
| | | localized to DSB to | | | | |
| | | initiate NHEJ | | | | |

TABLE 2

| | Olaparib | | | | | | | | |
|---|---|---|---|---|---|---|---|---|---|
| | SF50 | | | | | RNA- | Exon | | |
| Cell line | (uM) | COSMIC | SNP6 | RPPA | Methylation | seq | array | U133A | siRNA |
| BT20 | 50 | 1 | 1 | 1 | 1 | 1 | 1 | 1 | 1 |
| CAMA1 | 50 | 1 | 1 | 1 | 1 | 1 | 1 | 1 | 1 |
| HCC1428 | 50 | 0 | 1 | 1 | 1 | 1 | 1 | 1 | 0 |
| HCC38 | 50 | 1 | 1 | 1 | 1 | 1 | 1 | 1 | 0 |
| SKBR3 | 50 | 1 | 1 | 1 | 1 | 1 | 1 | 1 | 1 |
| BT474 | 31.99 | 1 | 1 | 1 | 1 | 1 | 1 | 1 | 1 |
| MDAMB134VI | 30.90 | 1 | 0 | 0 | 1 | 1 | 1 | 1 | 1 |
| MDAMB231 | 29.96 | 1 | 1 | 1 | 1 | 1 | 1 | 1 | 1 |
| BT549 | 21.43 | 1 | 1 | 1 | 1 | 1 | 1 | 1 | 0 |
| T47D | 19.95 | 1 | 1 | 1 | 1 | 1 | 1 | 1 | 1 |
| SUM159PT | 16.29 | 1 | 1 | 1 | 1 | 1 | 1 | 1 | 0 |
| HCC1954 | 15.49 | 1 | 1 | 1 | 1 | 1 | 1 | 1 | 0 |
| MCF7 | 14.69 | 1 | 1 | 1 | 1 | 1 | 1 | 1 | 1 |
| HS578T | 6.55 | 1 | 1 | 1 | 1 | 1 | 1 | 1 | 1 |
| MDAMB157 | 2.41 | 1 | 1 | 1 | 1 | 1 | 1 | 1 | 1 |
| HCC70 | 0.655 | 1 | 1 | 1 | 1 | 1 | 1 | 1 | 0 |
| MDAMB468 | 0.514 | 1 | 1 | 1 | 1 | 0 | 1 | 1 | 1 |
| HCC202 | 0.413 | 0 | 1 | 1 | 1 | 1 | 1 | 1 | 1 |
| HCC1143 | 0.0211 | 1 | 1 | 1 | 1 | 1 | 1 | 1 | 1 |
| SUM149PT | 0.0161 | 1 | 1 | 1 | 1 | 1 | 1 | 1 | 1 |
| MDAMB453 | 0.00915 | 1 | 1 | 1 | 1 | 1 | 1 | 1 | 1 |
| MDAMB436 | 0.00044 | 1 | 1 | 1 | 1 | 0 | 1 | 1 | 0 |
| # cell lines | | 20 | 21 | 21 | 22 | 20 | 22 | 22 | 15 |

TABLE 3

| | | | Promoter | |
|---|---|---|---|---|
| Mutation | Expression/protein level | Copy number | methylation | siRNA |
| BRCA1/2(−) | ESR1(−), PGR, ERBB2 | BRCA1 LOH | BRCA1(+) | ATM(−) |
| PTEN(−) | BER: PARP1/2(+), APEX1, | PARP1 ampl | FANCF(+) | ATR(−) |
| | XRCC1, LIG3, POLB, PAR(−) | | | |
| PALB2(−) | HR: BRCA1/2(−), PTEN(−), | Incr. genomic | | CHEK1(−) |
| | RAD50, RAD51(−), RAD54(−), | aberrations | | |
| | NBS1(−), ERCC1, XRCC3, | | | |
| | FANCF, TP53BP1(+), USP11(−), | | | |
| | DSS1(−), RPA1(−) | | | |
| ATM(−) | DDR: ATM(−), ATR(−), | BRCA1-related | | CDK5(−) |
| | CHEK1(+), CHEK2(−) | aCGH profile | | |
| CHEK1(−) | FA/BRCA pathway: FANCA, | EMSY ampl | | MAPK12(−) |
| | FANCC, FANCE, FANCG, | | | |
| | FANCD2, FANCL | | | |
| ATR(−) | VPARP, TNKS, TNKS2 | c-MYC ampl | | PLK3(−) |
| CHEK2(−) | HMGA1(+), ID4(+), POLQ | AURKA ampl | | PNKP(−) |
| MRE11A(−) | γH2AX(+) | | | STK22C(−) |
| NBS1(−) | | | | STK36(−) |
| TP53(−) | | | | |

(−)mutation/deficiency/down-regulation results in PARPi sensitivity
(+)up-regulation/promoter methylation results in PARPi sensitivity

TABLE 4a

| | | Response | Nb of | |
|---|---|---|---|---|
| | | in mutated | mu- | |
| | P- | vs. wt | tated | |
| Gene | value | lines | lines | Mutated lines |
| BRCA1 | 0.037 | sensitive | 2/20 | MDAMB436, SUM149PT |
| PTEN | 0.511 | sensitive | 5/20 | BT549, CAMA1, HCC70, |
| | | | | MDAMB453, MDAMB468 |
| BRCA1/ | 0.051 | sensitive | 7/20 | BT549, CAMA1, HCC70, |
| PTEN | | | | MDAMB436, MDAMB453, |
| | | | | MDAMB468, SUM149PT |
| TP53 | 0.521 | resistant | 13/16 | BT20, BT474, BT549, |
| | | | | CAMA1, HCC1143, HCC1954, |
| | | | | HCC38, HCC70, HS578T, |
| | | | | MDAMB157, MDAMB231, |
| | | | | MDAMB468, T47D |

TABLE 4b

| | P-value U133A | Expr S vs. | P-value U133A | Expr S vs. | P-value | Expr S vs. | P-value | Expr S vs. |
|---|---|---|---|---|---|---|---|---|
| Gene | standard | R lines | custom | R lines | exon array | R lines | RNA-seq | R lines |
| APEX1 | 0.593 | − | 0.593 | − | 0.061 | − | 0.178 | − |
| ATM | 1 | | 0.640 | +(45) | 0.841 | + | 0.267 | − |
| ATR | 1 | | 1 | | 0.947 | − | 0.428 | − |
| AURKA | 0.182 | − | 0.229 | − | 0.013 | − | 0.004 | − |
| BRCA1 | 0.285 | − | 0.216 | − | 0.463 | − | 0.048 | − |
| BRCA2 | 0.841 | +(100) | 0.548 | +(100) | 0.142 | + | 0.579 | −(40) |
| c-MYC | 0.504 | − | 0.463 | − | 0.789 | + | 0.937 | |
| c-MYC | 0.504 | − | 0.463 | − | 0.789 | + | 0.937 | |
| CDK5 | 0.033 | + | 0.027 | + | 0.35 | + | 0.205 | + |
| CHEK1 | 0.593 | +(50) | 0.841 | +(32) | 0.385 | + | 0.267 | − |
| CHEK2 | 0.038 | + | 0.003 | + | 0.35 | + | 0.751 | − |
| DSS1 | 0.789 | | 0.841 | | 0.504 | − | 0.579 | − |
| EMSY | 0.071 | −(95) | 0.095 | −(95) | 0.385 | − | 0.303 | − |
| ERBB2 | 0.504 | − | 0.689 | − | 0.182 | − | 0.579 | − |
| ERCC1 | 0.947 | | 0.947 | + | 0.285 | − | 0.132 | + |
| ESR1 | 0.062 | −(68) | 0.109 | − | 0.071 | − | 0.937 | −(65) |
| FANCA | 0.35 | − | 0.64 | − | 0.789 | + | 1 | |
| FANCC | 0.504 | − | 0.385 | − | 0.689 | + | 0.874 | + |
| FANCD2 | n/a | n/a | n/a | n/a | 0.463 | − | 0.081 | − |
| FANCE | 0.463 | + | 0.504 | + | 0.142 | + | 0.526 | |
| FANCF | 1 | | 0.894 | | 0.593 | − | 0.205 | + |
| FANCG | 0.256 | + | 0.35 | + | 0.504 | | 1 | |
| FANCL | 0.205 | + | 0.161 | + | 0.256 | + | 0.476 | |
| γH2AX | 0.204 | − | 0.071 | − | 0.053 | − | 0.692 | + |
| HMGA1 | 0.463 | + | 0.229 | + | 0.385 | + | 0.048 | + |
| ID4 | 0.789 | +(73) | 0.548 | +(73) | 0.463 | +(41) | 0.874 | +(65) |
| LIG3 | 0.64 | − | 0.256 | − | 0.204 | − | 0.751 | + |
| MAPK12 | 0.385 | + | 0.548 | + | 0.229 | + | 0.303 | + |
| MRE11A | 0.423 | − | 0.423 | − | 0.061 | − | 0.057 | − |
| NBS1 | 0.35 | − | 0.182 | − | 0.229 | − | 0.113 | − |
| PALB2 | 0.947 | | 1 | | 0.738 | | 0.113 | − |
| PAR | 0.841 | + | 0.894 | | 0.689 | + | 0.812 | |
| PARP1 | 0.789 | + | 0.789 | + | 0.463 | + | 0.579 | + |
| PARP2 | 0.434 | + | 0.947 | + | 0.947 | | 0.692 | + |
| PGR | 0.142 | −(91) | 0.109 | −(91) | 0.082 | −(68) | 0.069 | −(80) |
| PLK3 | 0.841 | | 0.947 | | 0.161 | + | 0.428 | + |
| PNKP | 0.894 | | 0.789 | | 0.789 | − | 0.026 | + |
| POLB | 0.738 | + | 0.688 | + | 0.64 | − | 0.235 | − |
| POLQ | 0.947 | | 0.947 | | 0.593 | − | 0.428 | − |
| PTEN | 0.894 | | 0.640 | −(50) | 0.423 | − | 0.154 | − |
| RAD50 | 0.640 | + | 0.504 | + | 0.841 | + | 0.579 | − |
| RAD51 | 0.593 | − | 0.182 | − | 1 | | 1 | − |
| RAD54 | 0.548 | + | 0.463 | +(55) | 0.947 | − | 0.634 | +(100) |
| RPA1 | 0.841 | | 0.689 | + | 0.385 | − | 0.428 | − |
| STK22C | n/a | n/a | n/a | n/a | 0.35 | + | 0.057 | + |
| STK36 | n/a | n/a | n/a | n/a | 0.548 | − | 0.383 | − |
| TNKS | 0.548 | −(32) | 0.463 | −(41) | 0.463 | − | 0.178 | − |
| TNKS2 | 0.504 | − | 0.385 | − | 0.256 | − | 0.004 | − |
| TP53 | 0.204 | − | 0.182 | − | 0.385 | − | 0.579 | − |
| TP53BP1 | 0.947 | | 1 | | 0.947 | | 0.579 | − |
| USP11 | 0.738 | | 0.738 | | 0.947 | | 0.937 | − |
| VPARP | 0.894 | + | n/a | n/a | 0.689 | | 0.526 | −(25) |
| XRCC1 | 0.738 | − | 0.593 | − | 0.689 | | 0.113 | + |
| XRCC3 | 0.526 | − | 0.35 | − | 1 | | 0.011 | + |

−: down-regulation in the sensitive w.r.t. resistant cell lines;
+: up-regulation in the sensitive w.r.t. resistant cell lines;
n/a: gene not measured on the specific platform

TABLE 4c

| | | CNV in sensitive vs. |
|---|---|---|
| Gene | P-value | resistant lines |
| BRCA1 | 0.012 | deletion |
| PARP1 | 0.166 | amplification |
| EMSY | 0.110 | deletion |
| c-MYC | 0.145 | less amplified |
| AURKA | 0.214 | less amplified |

TABLE 4d

| | Position | | # CG | # | Methylation |
|---|---|---|---|---|---|
| | meth. | P- | dinucle- | off-CpG | in sens. vs. |
| Gene | probe | value | otides | cytosines | res. lines |
| BRCA1 | 38,507,849 | 0.068 | 2 | 10 | hypo |
| (17q21) | 38,526,034 | 0.068 | 2 | 6 | hypo |
| 38,449,840- | 38,526,965 | 0.692 | 2 | 8 | hypo |
| 38,530,994 | 38,530,585 | 0.476 | 1 | 13 | hypo |
| | 38,530,739 | 0.154 | 2 | 21 | hypo |
| | 38,530,848 | 0.812 | 2 | 18 | slightly hypo |
| | 38,530,970 | 0.812 | 3 | 12 | similar |
| | 38,532,148 | 0.874 | 3 | 8 | slightly hypo |
| | 38,532,181 | 0.428 | 5 | 15 | slightly hyper |
| FANCF | 22,603,173 | 0.738 | 3 | 9 | slightly hyper |
| (11p15) | 22,603,297 | 0.947 | 3 | 13 | slightly hyper |
| 22,600,655- | 22,603,507 | 0.548 | 2 | 12 | slightly hypo |
| 22,603,963 | 22,603,699 | 0.229 | 4 | 13 | hypo |
| | 22,603,885 | 0.229 | 5 | 7 | slightly hypo |
| | 22,604,062 | 0.463 | 3 | 7 | slightly hypo |

TABLE 4e

| | | Loss of viability in sensitive vs. |
|---|---|---|
| siRNA | P-value | resistant lines |
| ATM | 0.152 | Less loss of viability |
| ATR | 0.694 | Less loss of viability |
| CHEK1 | 0.232 | More loss of viability |
| CDK5 | 0.535 | More loss of viability |
| MAPK12 | 0.152 | Less loss of viability |
| PLK3 | 0.779 | Less loss of viability |
| PNKP | 0.463 | Less loss of viability |
| STK22C | 0.142 | More loss of viability |
| STK36 | 0.866 | |

TABLE 5

| Biomarker | | | | Avg. test | Avg. test |
|---|---|---|---|---|---|
| source | Platform | # genes | Genes selected in >250/500 iterations | AUC (std)* | AUC (std)\hat{ } |
| Literature | U133A | 6/29 | BRCA1, ATM, CHEK1, | 0.602 | 0.692 |
| (Wang et al, | (standard) | | CHEK2, MRE11A, TP53 | (0.079) | (0.081) |
| 2011) | U133A | 7/29 | BRCA1, BRCA2, RAD51, | 0.816 | 0.611 |
| | (custom) | | XRCC5, ATR, CHEK2, | (0.066) | (0.072) |
| | | | γH2AX | | |
| | Exon array | 9/29 | BRCA2, FANCD2, RPA1, | 0.678 | 0.617 |
| | | | USP11, XPA, CHEK1, | (0.063) | (0.079) |
| | | | γH2AX, MAPKAPK2, NBS1 | | |
| | RNA-seq | 10/29 | BRCA1, FANCD2, PALB2, | 0.626 | 0.490 |
| | | | XPA, XRCC5, XRCC6, | (0.094) | (0.066) |
| | | | ATM, CHEK1, CHEK2, | | |
| | | | MRE11A | | |
| KEGG | U133A | 11/103 | POLE, RAD54L, TOP3B, | 0.745 | 0.573 |
| | (standard) | | RAD23A, RAD23B, DNTT, | (0.094) | (0.055) |
| | | | NHEJ1, POLM, XRCC5, | | |
| | | | XRCC6, RPA2 | | |
| | U133A | 13/103 | PARP3, POLE, POLE3, | 0.675 | 0.545 |
| | (custom) | | RAD51, RAD54L, RAD23B, | (0.086) | (0.050) |
| | | | DNTT, FEN1, NHEJ1, | | |
| | | | POLM, XRCC5, RFC3, | | |
| | | | RPA2 | | |
| | Exon array | 5/103 | TDG, MRE11A, CDK7, | 0.987 | 0.953 |
| | | | PRKDC, RPA2 | (0.030) | (0.060) |
| | RNA-seq | 5/103 | TDG, MUS81, POLD1, | 0.902 | 0.798 |
| | | | XRCC5, XRCC6 | (0.054) | (0.107) |

*Results with optimized LR coefficients and inclusion of all genes selected in >½ of the iterations
\hat{ }Results with +/−1 LR coefficients and inclusion of all genes selected in >½ of the iterations

TABLE 6

| | | # | # predicted | | Jaccard |
|---|---|---|---|---|---|
| Data set | Platform | samples | responders (%) | | coefficient |
| GSE2034 | U133A | 286 | 133 | (46.5) | 0.536 |
| GSE20271 | U133A | 177 | 78 | (44.1) | 0.429 |
| GSE23988 | U133A | 61 | 29 | (47.5) | 0.571 |
| GSE4922 | U133A + B | 289 | 121 | (41.9) | 0.464 |
| GSE1456 | U133A + B | 159 | 66 | (41.5) | 0.5 |
| GSE7390 | U133A | 198 | 91 | (46.0) | 0.5 |
| GSE11121 | U133A | 200 | 91 | (45.5) | 0.643 |
| GSE12093 | U133A | 136 | 65 | (47.8) | 0.75 |
| GSE23177 | U133 plus 2 | 116 | 47 | (40.5) | 0.5 |
| GSE5460 | U133 plus 2 | 127 | 63 | (49.6) | 0.536 |
| I-SPY1 | U133A | 117 | 48 | (41.0) | 0.464 |
| TCGA | Agilent G4502A | 430 | 185 | (43.0) | 0.714 |

TABLE 7

| | Non-re- | Re- | | | Non-re- | Re- |
|---|---|---|---|---|---|---|
| | sponders | sponders | | | sponders | sponders |
| I-SPY1 | N (%) | N (%) | | TCGA | N (%) | N (%) |
| Luminal A | 17 (25.4) | 15 | (35.7) | Luminal A | 99 (41.3) | 88 (48.3) |
| Luminal B | 17 (25.4) | 5 | (11.9) | Luminal B | 73 (30.4) | 36 (19.8) |
| Basal | 22 (32.8) | 19 | (45.2) | Basal | 37 (15.4) | 42 (23.1) |
| ERBB2 | 11 (16.4) | 3 | (7.1) | ERBB2 | 31 (12.9) | 16 (8.8) |
| amplified | | | | amplified | | |

| | | | |
|---|---|---|---|
| P-value | 0.1094 | P-value | 0.0145 |
| Chi-square | | Chi-square | |
| test | | test | |

TABLE 9

| | olapa- | Doubling | | | | | | | | | | |
|---|---|---|---|---|---|---|---|---|---|---|---|---|
| | rib SF50 | time | | | ERB | COS- | | RPP | | RNA- | Exon | |
| Cell line | (μm) | (hrs) | ER^{a} | PR^{a} | B2^{a} | MIC | SNP6 | A | Methylation | seq | array | U133A |
| HCC1428 | 50 | 88.5 | + | + | − | N | Y | Y | Y | Y | Y | Y |
| SKBR3 | 50 | 56.2 | − | + | + | Y | Y | Y | Y | Y | Y | Y |
| BT20 | 50 | 66.1 | − | NC | − | Y | Y | Y | Y | Y | Y | Y |
| HCC38 | 50 | 51.0 | − | − | − | Y | Y | Y | Y | Y | Y | Y |
| CAMA1 | 50 | 72.9 | + | NC | NC | Y | Y | Y | Y | Y | Y | Y |
| BT474 | 31.99 | 92.5 | − | − | − | Y | Y | Y | Y | Y | Y | Y |
| MDAMB134 | 30.90 | 82.7 | + | + | − | Y | N | N | Y | Y | Y | Y |
| VI | | | | | | | | | | | | |
| MDAMB231 | 29.96 | 25.0 | − | − | − | Y | Y | Y | Y | Y | Y | Y |
| BT549 | 21.43 | 25.5 | − | − | + | Y | Y | Y | Y | Y | Y | Y |
| T47D | 19.95 | 55.8 | + | + | NC | Y | Y | Y | Y | Y | Y | Y |
| SUM159PT | 16.29 | 21.7 | − | + | − | Y | Y | Y | Y | Y | Y | Y |
| HCC1954 | 15.49 | 43.8 | − | − | − | Y | Y | Y | Y | Y | Y | Y |
| MCF7 | 14.69 | 56.5 | − | − | − | Y | Y | Y | Y | Y | Y | Y |
| HS578T | 6.55 | 32.3 | − | − | − | Y | Y | Y | Y | Y | Y | Y |
| MDAMB157 | 2.41 | 67.0 | − | + | + | Y | Y | Y | Y | Y | Y | Y |
| HCC70 | 0.655 | 67.8 | − | − | NC | Y | Y | Y | Y | Y | Y | Y |
| MDAMB468 | 0.514 | 79.8 | − | − | − | Y | Y | Y | Y | N | Y | Y |
| HCC202 | 0.413 | 212.5 | − | NC | NC | N | Y | Y | Y | Y | Y | Y |
| HCC1143 | 0.0211 | 54.6 | − | − | − | Y | Y | Y | Y | Y | Y | Y |
| SUM149PT | 0.0161 | 33.9 | + | + | − | Y | Y | Y | Y | Y | Y | Y |
| MDAMB453 | 0.00915 | 62.5 | − | + | + | Y | Y | Y | Y | Y | Y | Y |
| MDAMB436 | 0.00044 | 89.3 | − | NC | − | Y | Y | Y | Y | N | Y | Y |
| # cell lines | | | | | | 20 | 21 | 21 | 22 | 20 | 22 | 22 |

^{a}For ER, probe 205225_at on the Affymetrix U133A array was investigated; for PR, probe 208305_at; and for ERBB2 probes 210930_s_at and 216836_s_at

TABLE 10

| | | | | Avg. |
|---|---|---|---|---|
| Biomarker | | | | AUC |
| source | Platform | # genes | Genes selected in >250/500 iterations^{a} | (std)^{b} |
| DNA repair | U133A | 11/29 | BRCA1, BRCA2, CHEK2, DSS1, | 0.793 |
| biomarkers | (standard) | | MRE11A, NBS1, PALB2, PARP2, PTEN, | (0.083) |
| (Wang et al, | | | TP53, XPA | |
| 2011) | U133A | 7/29 | BRCA1, BRCA2, CHEK2, DSS1, NBS1, | 0.945 |
| | (custom) | | RAD51, XPA | (0.059) |
| | Exon array | 12/29 | BRCA2, CHEK2, DSS1, ERCC1, ERCC4, | 0.717 |
| | | | FANCD2, MK2, MRE11A, NBS1, USP11, | (0.084) |
| | | | XPA, XRCC5 | |
| | RNA-seq | 14/29 | ATM, BRCA1, DSS1, FANCD2, JTB, | 0.715 |
| | | | MK2, MRE11A, NBS1, PALB2, PARP1, | (0.132) |
| | | | PARP2, XPA, XRCC5, XRCC6 | |
| KEGG | U133A | 5/103 | DNTT, MUTYH, POLM, RPA2, TOP3B | 0.745 |
| | (standard) | | | (0.075) |
| | U133A | 9/103 | DNTT, FEN1, MUTYH, NBS1, POLD1, | 0.725 |
| | (custom) | | POLM, RAD51, RAD51C, XRCC5 | (0.092) |
| | Exon array | 4/103 | DNTT, MRE11A, TDG, UNG | 0.753 |
| | | | | (0.083) |
| | RNA-seq | 5/103 | DCLRE1C, FEN1, RPA4, TDG, XRCC5 | 0.839 |
| | | | | (0.054) |

^{a}Genes with consistent pattern of sensitivity for all three platforms (U133A, exon array, RNA-seq) and for both measures of class comparison (mean, median) are shown in bold
^{b}Average 5-fold CV area under the receiver operating characteristics curve (AUC) (standard deviation) across 100 randomizations for a logistic regression model with optimized coefficients and inclusion of the platform-specific genes selected in >½ of the iterations

TABLE 11

| Gene | Gene | Entrez | | | Weight | Decision |
|---|---|---|---|---|---|---|
| symbol | name | gene ID | Marker | Probe | w_{g} | boundary b_{g} |
| BRCA1 | breast cancer 1, early | 672 | Resistance | 204531_s_at | −0.5320 | −0.0153 |
| | onset | | | | | |
| CHEK2 | CHK2 checkpoint | 11200 | Sensitivity | 210416_s_at | 0.5806 | −0.0060 |
| | homolog | | | | | |
| MK2 | mitogen-activated pro- | 9261 | Sensitivity | 201461_s_at | 0.0713 | 0.0031 |
| | tein kinase-activated | | | | | |
| | protein kinase 2 | | | | | |
| MRE11A | MRE11 meiotic | 4361 | Resistance | 205395_s_at | −0.1396 | −0.0044 |
| | recombination 11 | | | | | |
| | homolog A | | | | | |
| NBS1 | nibrin | 4683 | Resistance | 202906_s_at | −0.1976 | 0.0014 |
| TDG | thymine-DNA | 6996 | Resistance | 203743_s_at | −0.3937 | −0.0165 |
| | glycosylase | | | | | |
| XPA | Xeroderma pigmentosum, | 7507 | Resistance | 205672_at | −0.2335 | −0.0126 |
| | complementation | | | | | |
| | group A | | | | | |

TABLE 12

| | | # | | | Event | # predicted |
|---|---|---|---|---|---|---|
| Data set | Platform | samples | Characteristics | Treatment | rate, % | responders (%)* |
| GSE2034 | U133A | 286 | 73.1% ER+ | Untreated | 37.4% | 55 (19.2) |
| | | | 58% PR+ | | distant | |
| | | | 18.2% ERBB2+ | | metastasis | |
| | | | 0% LN+ | | | |
| GSE20271 | U133A | 177 | 55.7% ER+ | 49.2% | 14.1% | 26 (14.7) |
| | | | 46.9% PR+ | FAC | pCR | |
| | | | 14.2% ERBB2+ | 50.8% | | |
| | | | | T/FAC | | |
| GSE23988 | U133A | 61 | 52.5% ER+ | FEC/wTx | 32.8% | 9 (14.8) |
| | | | 0% ERBB2+ | | pCR | |
| | | | 65.6% LN+ | | | |
| | | | Median tumor size | | | |
| | | | 6 cm (2-17.5) | | | |
| GSE4922 | U133A + B | 289 | 86.1% ER+ | 37.7% | 35.7% | 24 (8.3) |
| | | | 33.7% LN+ | systematic | local/ | |
| | | | Median tumor size | adjuvant | distant | |
| | | | 2 cm (0.2-13) | therapy | recurrence | |
| | | | | | or death | |
| GSE25066 | U133A | 508 | 58.9% ER+ | Neoadj. | 19.5% | 94 (18.5) |
| | | | 69.1% LN+ | taxane & | pCR | |
| | | | 31.5% lumA | anthra- | | |
| | | | 15.3% lumB | cycline- | | |
| | | | 37.2% basal-like | based | | |
| | | | 7.3% HER2-enr | regimen | | |
| | | | 8.7% normal-like | | | |
| GSE7390 | U133A | 198 | 67.7% ER+ | Untreated | 31.3% | 33 (16.7) |
| | | | 14.1% ERBB2+ | | distant | |
| | | | 0% LN+ | | metastasis | |
| | | | Median tumor size | | | |
| | | | 2 cm (0.6-5) | | | |
| GSE11121 | U133A | 200 | 78% ER+ | Untreated | 23% | 20 (10.0) |
| | | | 65% PR+ | | distant | |
| | | | 12.3% ERBB2+ | | metastasis | |
| | | | 0% LN+ | | | |
| | | | Median tumor size | | | |
| | | | 2 cm (0.1-6.0) | | | |
| GSE5460 | U133 plus | 127 | 58.3% ER+ | Untreated | — | 27 (21.3) |
| | 2 | | 23.6% ERBB2+ | | | |
| | | | 49.6% LN+ | | | |
| | | | Median tumor size | | | |
| | | | 2.2 cm (0.8-8.5) | | | |
| TCGA | Agilent | 536 | 44.0% lumA | Hetero- | — | 67 (12.5) |
| | G4502A | | 25.2% lumB | geneous | | |
| | | | 18.5% basal-like | | | |
| | | | 10.8% HER2-enr | | | |
| | | | 1.5% normal-like | | | |

*Number and percentage of patients predicted to respond to treatment with a PARP inhibitor according to the 7-gene predictor with use of threshold 0.0372 for response assignment for Affymetrix data, and threshold 0.174 for Agilent data
FAC = Neoadjuvant chemotherapy regimen with 5-fluorouracil, docorubicin and cyclophosphamide
T/FAC = Neoadjuvant chemotherapy regimen with paclitaxel and 5-fluorouracil, docorubicin and cyclophosphamide
FEC/wTx = Neoadjuvant chemotherapy regimen with four courses of 5-fluorouracil, docorubicin and cyclophosphamide, followed by four additional courses of weekly docetaxel and capecitabine

TABLE 13

| | Non-responders | | Responders | | | Non-responders | | Responders | |
|---|---|---|---|---|---|---|---|---|---|
| GSE25066 | N (%) | | N (%) | | TCGA | N (%) | | N (%) | |
| Luminal A | 120 | (75.0) | 40 | (25.0) | Luminal A | 233 | (98.7) | 3 | (1.3) |
| Luminal B | 72 | (92.3) | 6 | (7.7) | Luminal B | 126 | (93.3) | 9 | (6.7) |
| Basal-like | 155 | (82.0) | 34 | (18.0) | Basal-like | 54 | (54.5) | 45 | (45.5) |
| HER2-enriched | 35 | (94.6) | 2 | (5.4) | HER2-enriched | 50 | (86.2) | 8 | (13.8) |

| | | | |
|---|---|---|---|
| P-value | 0.002 | P-value | 2.6 × 10⁻²⁸ |
| Chi-square test | | Chi-square test | |

TABLE 14a

| | P-value | | P-value | | | | | |
|---|---|---|---|---|---|---|---|---|
| | U133A | FC S vs. | U133A | FC S vs. | P-value | FC S vs. | P-value | FC S vs. |
| Gene | standard | R lines | custom | R lines | exon array | R lines | RNA-seq | R lines |
| ATM | 0.778 | −1.01 | 0.888 | −1.02 | 0.204 | −1.56 | 0.162 | −1.86 |
| ATR | 0.672 | 1.47 | 0.622 | 1.34 | 0.672 | −1.20 | 0.295 | −1.51 |
| BRCA1 | 0.180 | −1.27 | 0.129 | −1.31 | 0.078 | −1.66 | 0.055 | −2.09 |
| BRCA2 | 0.438 | 1.08 | 0.204 | 1.09 | 0.204 | 1.78 | 0.793 | −1.40 |
| CHEK1 | 0.573 | 1.26 | 0.672 | 1.35 | 0.622 | 1.14 | 0.295 | −1.45 |
| CHEK2 | 0.014 | 1.47 | 0.001 | 1.75 | 0.024 | 1.48 | 0.861 | 1.50 |
| DSS1 | 0.139 | −1.41 | 0.139 | −1.42 | 0.139 | −1.28 | 0.727 | 1.09 |
| ER | 0.204 | −22.21 | 0.139 | −1.45 | 0.398 | −9.80 | 0.600 | −659.5 |
| ERBB2 | 0.888 | 1.18 | 0.724 | −1.01 | 0.672 | −1.34 | 0.662 | 1.09 |
| ERCC1 | 1 | −1.11 | 1 | −1.14 | 0.259 | −1.32 | 0.295 | 1.10 |
| ERCC4 | 0.359 | −1.09 | 0.324 | −1.11 | 0.290 | −1.32 | 0.081 | −1.73 |
| FANCD2 | n/a | n/a | n/a | n/a | 0.139 | −1.31 | 0.067 | −1.77 |
| γH2AX | 0.204 | −1.30 | 0.105 | −1.32 | 0.259 | −1.20 | 0.930 | 1.63 |
| JTB | 0.105 | 1.24 | 0.139 | 1.16 | 0.121 | 1.22 | 0.485 | 1.14 |
| LIG3 | 0.888 | 1.04 | 0.526 | −1.08 | 0.481 | −1.11 | 1 | 1.46 |
| MK2 | 0.259 | 1.59 | 0.159 | 1.00 | 0.024 | 1.38 | 0.067 | 1.50 |
| MLH1 | 0.724 | −1.04 | 0.573 | −1.10 | 0.231 | −1.33 | 0.793 | −1.40 |
| MRE11A | 0.622 | −1.30 | 0.672 | −1.21 | 0.041 | −2.00 | 0.295 | −2.13 |
| NBS1 | 0.078 | −2.27 | 0.034 | −2.56 | 0.048 | −2.08 | 0.097 | −2.31 |
| PALB2 | 0.481 | 1.49 | 0.573 | 1.50 | 0.832 | 1.08 | 0.162 | −1.37 |
| PAR | 0.778 | −1.02 | 0.231 | −1.09 | 1 | 1.04 | 0.924 | −1.14 |
| PARP1 | 0.259 | 1.30 | 0.231 | 1.33 | 0.359 | 1.14 | 0.295 | 1.28 |
| PARP2 | 0.091 | 1.82 | 0.324 | 1.48 | 0.944 | 1.17 | 0.727 | −1.15 |
| PR | 0.139 | −3.57 | 0.105 | −3.53 | 0.105 | −29.65 | 0.076 | −232.0 |
| PRKDC | 0.526 | −1.11 | 0.944 | −1.11 | 1 | 1.05 | 0.727 | 1.06 |
| PTEN | 0.438 | −1.26 | 0.398 | −1.15 | 0.481 | −1.14 | 0.138 | −1.89 |
| RAD51 | 0.832 | 1.15 | 0.888 | 1.06 | 0.888 | 1.03 | 0.727 | 1.23 |
| RAD54 | 0.573 | 1.42 | 0.573 | 1.09 | 0.778 | −1.19 | 0.485 | −1.11 |
| RPA1 | 0.622 | 1.17 | 0.398 | 1.09 | 0.359 | −1.30 | 0.337 | −1.41 |
| TNKS | 0.438 | −1.73 | 0.438 | −1.13 | 0.259 | −1.29 | 0.014 | −2.87 |
| TNKS2 | 0.778 | 1.01 | 0.944 | −1.02 | 0.724 | −1.00 | 0.023 | −2.46 |
| TP53 | 0.724 | −1.22 | 0.672 | −1.22 | 1 | 1.23 | 0.930 | 1.46 |
| TP53BP1 | 0.724 | 1.14 | 0.724 | 1.13 | 0.481 | −1.10 | 0.793 | −1.21 |
| USP11 | 0.888 | −1.55 | 0.888 | −1.22 | 0.573 | −1.58 | 0.432 | −2.24 |
| VPARP | 0.778 | 1.17 | n/a | n/a | 1 | 1.10 | 0.930 | 1.39 |
| XPA | 0.078 | −1.43 | 0.078 | −1.43 | 0.011 | −1.72 | 0.067 | −2.35 |
| XRCC1 | 0.832 | −1.06 | 0.622 | −1.13 | 0.778 | −1.05 | 0.727 | 1.47 |
| XRCC2 | 0.398 | −1.08 | 0.724 | 1.03 | 0.204 | −1.30 | 0.162 | −1.66 |
| XRCC3 | 0.916 | 1.127 | 0.832 | 1.13 | 0.724 | 1.08 | 0.081 | 1.68 |
| XRCC5 | 0.438 | −1.12 | 0.573 | −1.17 | 0.057 | −1.27 | 0.009 | −2.04 |
| XRCC6 | 1 | 1.04 | n/a | n/a | 0.778 | −1.01 | 0.861 | 1.20 |

n/a: gene not measured on the specific platform

TABLE 14b

| | | Nb of | Nb of | |
|---|---|---|---|---|
| | | sensi- | resis- | |
| | | tive | tant | |
| | P- | mutat- | mutat- | |
| Gene | value | ed lines | ed lines | Mutated lines |
| BRCA1 | 0.091 | 2/7 | 0/15 | MDAMB436, SUM149PT |
| PTEN | 0.145 | 4/7 | 3/15 | BT549, CAMA1, HCC38°, |
| defi- | | | | HCC70, MDAMB436°, |
| ciency | | | | MDAMB453, MDAMB468° |
| BRCA1/ | 0.052 | 5/7 | 3/15 | BT549, CAMA1, HCC38°, |
| PTEN | | | | HCC70, MDAMB436°, |
| defi- | | | | MDAMB453, MDAMB468°, |
| ciency | | | | SUM149PT |
| TP53 | 0.376 | 3/7 | 10/15 | BT20, BT474, BT549, CAMA1, |
| | | | | HCC1143, HCC1954, HCC38, |
| | | | | HCC70, HS578T, MDAMB157, |
| | | | | MDAMB231, MDAMB468, |
| | | | | T47D |

°PTEN null (no expression of PTEN protein and/or PTEN transcript)

TABLE 14c

| | | CNV in sensitive vs. |
|---|---|---|
| Gene | P-value | resistant lines |
| BRCA1 | 0.012 | deletion |
| PARP1 | 0.080 | amplification |
| PTEN | 0.526 | amplification |

TABLE 14d

| | Position | | # CG | # | Methylation |
|---|---|---|---|---|---|
| | meth. | P- | dinucle- | off-CpG | in sens. vs. |
| Gene | probe | value | otides | cytosines | res. lines |
| BRCA | 38,507,849 | 0.138 | 2 | 10 | hypo |
| (17q21) | 38,526,034 | 0.097 | 2 | 6 | hypo |
| 38,449,840- | 38,526,965 | 0.793 | 2 | 8 | slightly hypo |
| 38,530,994 | 38,530,585 | 0.663 | 1 | 13 | slightly hyper |
| | 38,530,739 | 0.163 | 2 | 21 | hypo |
| | 38,530,848 | 0.432 | 2 | 18 | hyper |
| | 38,530,970 | 0.485 | 3 | 12 | slightly hyper |
| | 38,532,148 | 0.930 | 3 | 8 | similar |
| | 38,532,181 | 0.727 | 5 | 15 | slightly hyper |
| FANCF | 22,603,173 | 0.324 | 3 | 9 | slightly hypo |
| (11p15) | 22,603,297 | 0.944 | 3 | 13 | similar |
| 22,600,655- | 22,603,507 | 0.231 | 2 | 12 | hypo |
| 22,603,963 | 22,603,699 | 0.078 | 4 | 13 | hypo |
| | 22,603,885 | 0.231 | 5 | 7 | slightly hypo |
| | 22,604,062 | 0.944 | 3 | 7 | similar |

TABLE 15

| | BER | NER | | HR | NHEJ | DDR |
|---|---|---|---|---|---|---|
| DNA repair | JTB | ERCC1 | | BRCA1 | PRKDC | ATM |
| biomarkers | PARP1 | ERCC4 | | BRCA2 | XRCC5 | ATR |
| (Wang et al, | PARP2 | XPA | | DSS1 | XRCC6 | CHEK1 |
| 2011) | | | | FANCD2 | | CHEK2 |
| | | | | PALB2 | | H2AFX |
| | | | | PTEN | | MK2 |
| | | | | RAD51 | | MRE11A |
| | | | | RAD54 | | NBS1 |
| | | | | RPA1 | | TP53 |
| | | | | TP53BP1 | | |
| | | | | USP11 | | |
| | BER | NER | | HR | NHEJ | MMR |
| | map03410 | map03420 | | map03440 | map03450 | map03430 |
| KEGG release | APEX1 | CCNH | POLD1 | BLM | DCLRE1C | EXO1 |
| 55.1 | APEX2 | CDK7 | POLD2 | BRCA2 | DNTT | LIG1 |
| | FEN1 | CETN2 | POLD3 | DSS1 | FEN1 | MLH1 |
| | HMGB1 | CUL4A | POLD4 | EME1 | LIG4 | MLH3 |
| | LIG1 | CUL4B | POLE | MRE11A | MRE11A | MSH2 |
| | LIG3 | DDB1 | POLE2 | MUS81 | NHEJ1 | MSH3 |
| | MBD4 | DDB2 | POLE3 | NBN | POLL | MSH6 |
| | MPG | ERCC1 | POLE4 | POLD1 | POLM | PCNA |
| | MUTYH | ERCC2 | RAD23A | POLD2 | PRKDC | PMS2 |
| | NEIL1 | ERCC3 | RAD23B | POLD3 | RAD50 | POLD1 |
| | NEIL2 | ERCC4 | RBX1 | POLD4 | XRCC4 | POLD2 |
| | NEIL3 | ERCC5 | RFC1 | RAD50 | XRCC5 | POLD3 |
| | NTHL1 | ERCC6 | RFC2 | RAD51 | XRCC6 | POLD4 |
| | OGG1 | ERCC8 | RFC3 | RAD51C | | RFC1 |
| | PARP1 | GTF2H1 | RFC4 | RAD51L1 | | RFC2 |
| | PARP2 | GTF2H2 | RFC5 | RAD51L3 | | RFC3 |
| | PARP3 | GTF2H3 | RPA1 | RAD52 | | RFC4 |
| | PARP4 | GTF2H4 | RPA2 | RAD54B | | RFC5 |
| | PCNA | GTF2H5 | RPA3 | RAD54L | | RPA1 |
| | POLB | LIG1 | RPA4 | RPA1 | | RPA2 |
| | POLD1 | MNAT1 | XPA | RPA2 | | RPA3 |
| | POLD2 | PCNA | XPC | RPA3 | | RPA4 |
| | POLD3 | | | RPA4 | | SSBP1 |
| | POLD4 | | | SSBP1 | | |
| | POLE | | | TOP3A | | |
| | POLE2 | | | TOP3B | | |
| | POLE3 | | | XRCC2 | | |
| | POLE4 | | | XRCC3 | | |
| | POLL | | | | | |
| | SMUG1 | | | | | |
| | TDG | | | | | |
| | UNG | | | | | |
| | XRCC1 | | | | | |

What is claimed is:

1. A method for predicting a cancer patient response to a PARP inhibitor, comprising: (a) measuring the amplification or expression level of one or more genes selected from the group consisting of the genes encoding BRCA1, H2AFX, MRE11A, TDG, XRCC5, BRCA2, CHEK1, CHEK2, MK2, NBS1 and XPA in a sample from the patient; and (b) comparing the amplification or expression level of said gene(s) from the patient with the amplification or expression level of the gene(s) in a normal tissue sample or a reference amplification or expression level,
whereby an decrease of amplification or expression of one gene selected from the group consisting of the genes encoding BRCA1, H2AFX, MRE11A, TDG, XRCC5, NBS1 and XPA, and/or a increase of amplification or expression of one gene selected from the group consisting of the genes encoding BRCA2, CHEK1, CHEK2 and MK2 indicates a patient that is sensitive to a PARP inhibitor and suitable for treatment with a PARP inhibitor; and
whereby an increase of amplification or expression of one gene selected from the group consisting of the genes encoding BRCA1, H2AFX, MRE11A, TDG, XRCC5, NBS1 and XPA, and/or a decrease of amplification or expression of one gene selected from the group consisting of the genes encoding BRCA2, CHEK1, CHEK2 and MK2 indicates a patient that is resistant to a PARP inhibitor.

2. The method of claim 1, further comprising (c) comparing the amplification or expression level of the gene in the normal tissue sample or a reference amplification expression level, or the average amplification or expression level in a panel of normal cell lines or cancer cell lines.

3. A method for identifying a cancer patient suitable for treatment with a PARP inhibitor compound, comprising (a) measuring the amplification or expression level of a gene in a sample from the patient, and (b) comparing the amplification or expression level of the gene in the normal tissue sample or a reference amplification expression level, or the average amplification or expression level in a panel of normal cell lines or cancer cell lines, whereby a decrease of amplification or expression of one gene selected from the group consisting of the genes encoding BRCA1, H2AFX, MRE11A, TDG, XRCC5, NBS1 and XPA, and/or a increase of amplification or expression of one gene selected from the group consisting of the genes encoding BRCA2, CHEK1, CHEK2 and MK2 indicates a patient that is sensitive to a PARP inhibitor.

4. A method for identifying a cancer patient suitable for treatment with a PARP inhibitor compound, comprising: (a) measuring amplification or expression levels of a gene selected from the group consisting of genes encoding H2AFX, MRE11A, TDG, XRCC5, CHEK1 and CHEK2 in a sample from the patient; and (b) comparing the amplification or expression level of the gene from the patient with amplification or expression level of the gene in a normal tissue sample or a reference expression level, wherein an increase of amplification or expression of the gene encoding CHEK1 or CHEK2 and/or a decrease of amplification or expression of the gene encoding H2AFX, MRE11A, TDG or XRCC5 indicates the patient will be suitable for treatment with the PARP inhibitor.

5. The method of claim 4, wherein step (a) measuring amplification or expression levels of at least two, three, four, five or more genes selected from the group consisting of genes encoding H2AFX, MRE11A, TDG, XRCC5, CHEK1 and CHEK2 in a sample from the patient.

6. The method of claim 4, wherein step (a) measuring amplification or expression levels of at least one gene from the resistant group (H2AFX, MRE11A, TDG and XRCC5) and one from the sensitive group (CHEK1 and CHEK2).

7. The method of claim 4, wherein step (a) measuring amplification or expression levels of at least one gene from the resistant group (H2AFX, MRE11A, TDG and XRCC5).

8. The method of claim 4, wherein step (a) measuring amplification or expression levels of at least one gene from the sensitive group (CHEK1 and CHEK2).

9. A method for identifying a cancer patient suitable for treatment with a PARP inhibitor compound, comprising: (a) measuring amplification or expression levels of a gene selected from the group consisting of genes encoding BRCA1, BRCA2, H2AFX, MRE11A, TDG, XRCC5, CHEK1 and CHEK2 in a sample from the patient; and (b) comparing the amplification or expression level of the gene from the patient with amplification or expression level of the gene in a normal tissue sample or a reference expression level, wherein an increase of amplification or expression of the gene encoding BRCA2, CHEK1 or CHEK2 and/or a decrease of amplification or expression of the gene encoding BRCA1, H2AFX, MRE11A, TDG or XRCC5 indicates the patient will be suitable for treatment with the PARP inhibitor.

10. The method of claim 9, wherein step (a) measuring amplification or expression levels of at least two, three, four, five, six, seven or more genes selected from the group consisting of genes encoding BRCA1, BRCA2, H2AFX, MRE11A, TDG, XRCC5, CHEK1 and CHEK2 in a sample from the patient.

11. The method of claim 9, wherein step (a) measuring amplification or expression levels of at least one gene from the resistant group (BRCA1, H2AFX, MRE11A, TDG and XRCC5) and one from the sensitive group (BRCA2, CHEK1 and CHEK2).

12. The method of claim 9, wherein step (a) measuring amplification or expression levels of at least one gene from the resistant group (BRCA1, H2AFX, MRE11A, TDG and XRCC5).

13. The method of claim 9, wherein step (a) measuring amplification or expression levels of at least one gene from the sensitive group (BRCA2, CHEK1 and CHEK2).

14. A method for identifying a cancer patient suitable for treatment with a PARP inhibitor compound, comprising: (a) measuring amplification or expression levels of a gene selected from the group consisting of genes encoding BRCA1, MRE11A, TDG, CHEK2, MK2, NBS1 and XPA in a sample from the patient; and (b) comparing the amplification or expression level of the gene from the patient with amplification or expression level of the gene in a normal tissue sample or a reference expression level, wherein an increase of amplification or expression of the gene encoding MK2 or CHEK2 and/or a decrease of amplification or expression of the gene encoding MRE11A, TDG, BRCA1, NBS1 or XPA indicates the patient will be suitable for treatment with the PARP inhibitor.

15. The method of claim 14, wherein step (a) measuring amplification or expression levels of at least two, three, four, five, six, or more genes selected from the group consisting of genes encoding BRCA1, MRE11A, TDG, CHEK2, MK2, NBS1 and XPA in a sample from the patient.

16. The method of claim 14, wherein step (a) measuring amplification or expression levels of at least one gene from the resistant group (BRCA1, MRE11A, TDG, NBS1 and XPA) and one from the sensitive group (MK2 and CHEK2).

17. The method of claim 14, wherein step (a) measuring amplification or expression levels of at least one gene from the resistant group (BRCA1, MRE11A, TDG, NBS1 and XPA).

18. The method of claim 14, wherein step (a) measuring amplification or expression levels of at least one gene from the sensitive group (MK2 and CHEK2).

19. A method for identifying a cancer patient suitable for treatment with a PARP inhibitor, comprising: (a) measuring the amplification or expression level of one gene selected from the group consisting of the genes encoding BRCA1, MRE11A, TDG and CHEK2 in a sample from the patient; (b) measuring the amplification or expression level of at least one different gene selected from the group consisting of the genes encoding BRCA1, H2AFX, MRE11A, TDG, XRCC5, BRCA2, CHEK1, CHEK2, MK2, NBS1 and XPA; and (c) comparing the amplification or expression level of said genes from the patient with the amplification or expression level of the genes in a normal tissue sample or a reference amplification or expression level.

20. The method of claim 19, wherein step (b) measuring amplification or expression levels of at least two, three, four, five, six, seven or more different genes selected from the group consisting of genes encoding BRCA1, H2AFX, MRE11A, TDG, XRCC5, BRCA2, CHEK1, CHEK2, MK2, NBS1 and XPA in a sample from the patient.

21. The method of claim 19, wherein step (b) measuring amplification or expression levels of at least one genes selected from the group consisting of genes encoding MK2, NBS1 and XPA in a sample from the patient.

22. The method of claim 19, wherein step (b) measuring amplification or expression levels of at least one genes selected from the group consisting of genes encoding H2AFX, XRCC5, BRCA2 and CHEK1 in a sample from the patient.

23. A method for identifying a cancer patient suitable for treatment with a PARP inhibitor, comprising: (a) measuring the amplification or expression level of the group of genes encoding BRCA1, MRE11A, TDG and CHEK2; (b) measuring the amplification or expression level of at least one gene selected from the group consisting of the genes encoding H2AFX, XRCC5, BRCA2, CHEK1, MK2, NBS1 and XPA in a sample from the patient; and (b) comparing the amplification or expression level of said genes from the patient with the amplification or expression level of the genes in a normal tissue sample or a reference amplification or expression level.

24. The method of claim 23, wherein step (b) measuring amplification or expression levels of at least two, three or more genes selected from the group consisting of genes encoding H2AFX, XRCC5, BRCA2, CHEK1, MK2, NBS1 and XPA in a sample from the patient.

25. The method of claim 23, wherein step (b) measuring amplification or expression levels of at least one genes selected from the group consisting of genes encoding MK2, NBS1 and XPA in a sample from the patient.

26. The method of claim 23, wherein step (b) measuring amplification or expression levels of at least one genes selected from the group consisting of genes encoding H2AFX, XRCC5, BRCA2 and CHEK1 in a sample from the patient.

27. The methods of any of claims 1, 3, 4, 9, 14, 19 and 23, further comprising a step of prescribing and administering an effective amount of a PARP inhibitor to the patient.


SEQUENCE LISTING (43 sequences)

SEQ ID NO: 1 - DNA, 7224 bp, Homo sapiens
gtaccttgatttcgtattctgagaggctgctgcttagcggtagccccttggtttccgtggcaacggaaaagcgcgggaattacagataaattaaaactgcgactgcgcggcgtgagctcgctgagacttcctggacgggggacaggctgtggggtttctcagataactgggcccctgcgctcaggaggccttcaccctctgctctgggtaaagttcattggaacagaaagaaatggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactgactgcagccagccacaggtacagagccacaggaccccaagaatgagcttacaaagtggcctttccaggccctgggagctcctctcactcttcagtccttctactgtcctggctactaaatattttatgtacatcagcctgaaaaggacttctggctatgcaagggtcccttaaagattttctgcttgaagtctcccttggaaatctgccatgagcacaaaattatggtaatttttcacctgagaagattttaaaaccatttaaacgccaccaattgagcaagatgctgattcattatttatcagccctattctttctattcaggctgttgttggcttagggctggaagcacagagtggcttggcctcaagagaatagctggtttccctaagtttacttctctaaaaccctgtgttcacaaaggcagagagtcagacccttcaatggaaggagagtgcttgggatcgattatgtgacttaaagtcagaatagtccttgggcagttctcaaatgttggagtggaacattggggaggaaattctgaggcaggtattagaaatgaaaaggaaacttgaaacctgggcatggtggctcacgcctgtaatcccagcactttgggaggccaaggtgggcagatcactggaggtcaggagttcgaaaccagcctggccaacatggtgaaaccccatctctactaaaaatacagaaattagccggtcatggtggtggacacctgtaatcccagctactcaggtggctaaggcaggagaatcacttcagcccgggaggtggaggttgcagtgagccaagatcataccacggcactccagcctgggtgacagtgagactgtggctcaaaaaaaaaaaaaaaaaaaggaaaatgaaactagaagagatttctaaaagtctgagatatatttgctagatttctaaagaatgtgttctaaaacagcagaagattttcaagaaccggtttccaaagacagtcttctaattcctcattagtaataagtaaaatgtttattgttgtagctctggtatataatccattcctcttaaaatataagacctctggcatgaatatttcatatctataaaatgacagatcccaccaggaaggaagctgttgctttctttgaggtgatttttttcctttgctccctgttgctgaaaccatacagcttcataaataattttgcttgctgaaggaagaaaaagtgtttttcataaacccattatccaggactgtttatagctgttggaaggactaggtcttccctagcccccccagtgtgcaagggcagtgaagacttgattgtacaaaatacgttttgtaaatgttgtgctgttaacactgcaaataaacttggtagcaaacacttccaaaaaaaaaaaaaaaaaa

SEQ ID NO: 2 - DNA, 7287 bp, Homo sapiens
gtaccttgatttcgtattctgagaggctgctgcttagcggtagccccttggtttccgtggcaacggaaaagcgcgggaattacagataaattaaaactgcgactgcgcggcgtgagctcgctgagacttcctggacgggggacaggctgtggggtttctcagataactgggcccctgcgctcaggaggccttcaccctctgctctgggtaaagttcattggaacagaaagaaatggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagattcgcatatacatggccaaaggaacaactccatgttttctaaaaggcctagagaacatatatcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactgactgcagccagccacaggtacagagccacaggaccccaagaatgagcttacaaagtggcctttccaggccctgggagctcctctcactcttcagtccttctactgtcctggctactaaatattttatgtacatcagcctgaaaaggacttctggctatgcaagggtcccttaaagattttctgcttgaagtctcccttggaaatctgccatgagcacaaaattatggtaatttttcacctgagaagattttaaaaccatttaaacgccaccaattgagcaagatgctgattcattatttatcagccctattctttctattcaggctgttgttggcttagggctggaagcacagagtggcttggcctcaagagaatagctggtttccctaagtttacttctctaaaaccctgtgttcacaaaggcagagagtcagacccttcaatggaaggagagtgcttgggatcgattatgtgacttaaagtcagaatagtccttgggcagttctcaaatgttggagtggaacattggggaggaaattctgaggcaggtattagaaatgaaaaggaaacttgaaacctgggcatggtggctcacgcctgtaatcccagcactttgggaggccaaggtgggcagatcactggaggtcaggagttcgaaaccagcctggccaacatggtgaaaccccatctctactaaaaatacagaaattagccggtcatggtggtggacacctgtaatcccagctactcaggtggctaaggcaggagaatcacttcagcccgggaggtggaggttgcagtgagccaagatcataccacggcactccagcctgggtgacagtgagactgtggctcaaaaaaaaaaaaaaaaaaaggaaaatgaaactagaagagatttctaaaagtctgagatatatttgctagatttctaaagaatgtgttctaaaacagcagaagattttcaagaaccggtttccaaagacagtcttctaattcctcattagtaataagtaaaatgtttattgttgtagctctggtatataatccattcctcttaaaatataagacctctggcatgaatatttcatatctataaaatgacagatcccaccaggaaggaagctgttgctttctttgaggtgatttttttcctttgctccctgttgctgaaaccatacagcttcataaataattttgcttgctgaaggaagaaaaagtgtttttcataaacccattatccaggactgtttatagctgttggaaggactaggtcttccctagcccccccagtgtgcaagggcagtgaagacttgattgtacaaaatacgttttgtaaatgttgtgctgttaacactgcaaataaacttggtagcaaacacttccaaaaaaaaaaaaaaaaaa

SEQ ID NO: 3 - DNA, 7132 bp, Homo sapiens
cttagcggtagccccttggtttccgtggcaacggaaaagcgcgggaattacagataaattaaaactgcgactgcgcggcgtgagctcgctgagacttcctggacgggggacaggctgtggggtttctcagataactgggcccctgcgctcaggaggccttcaccctctgctctggttcattggaacagaaagaaatggatttatctgctcttcgcgttgaagaagtacaaaatgtcattaatgctatgcagaaaatcttagagtgtcccatctgattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactgactgcagccagccacaggtacagagccacaggaccccaagaatgagcttacaaagtggcctttccaggccctgggagctcctctcactcttcagtccttctactgtcctggctactaaatattttatgtacatcagcctgaaaaggacttctggctatgcaagggtcccttaaagattttctgcttgaagtctcccttggaaatctgccatgagcacaaaattatggtaatttttcacctgagaagattttaaaaccatttaaacgccaccaattgagcaagatgctgattcattatttatcagccctattctttctattcaggctgttgttggcttagggctggaagcacagagtggcttggcctcaagagaatagctggtttccctaagtttacttctctaaaaccctgtgttcacaaaggcagagagtcagacccttcaatggaaggagagtgcttgggatcgattatgtgacttaaagtcagaatagtccttgggcagttctcaaatgttggagtggaacattggggaggaaattctgaggcaggtattagaaatgaaaaggaaacttgaaacctgggcatggtggctcacgcctgtaatcccagcactttgggaggccaaggtgggcagatcactggaggtcaggagttcgaaaccagcctggccaacatggtgaaaccccatctctactaaaaatacagaaattagccggtcatggtggtggacacctgtaatcccagctactcaggtggctaaggcaggagaatcacttcagcccgggaggtggaggttgcagtgagccaagatcataccacggcactccagcctgggtgacagtgagactgtggctcaaaaaaaaaaaaaaaaaaaggaaaatgaaactagaagagatttctaaaagtctgagatatatttgctagatttctaaagaatgtgttctaaaacagcagaagattttcaagaaccggtttccaaagacagtcttctaattcctcattagtaataagtaaaatgtttattgttgtagctctggtatataatccattcctcttaaaatataagacctctggcatgaatatttcatatctataaaatgacagatcccaccaggaaggaagctgttgctttctttgaggtgatttttttcctttgctccctgttgctgaaaccatacagcttcataaataattttgcttgctgaaggaagaaaaagtgtttttcataaacccattatccaggactgtttatagctgttggaaggactaggtcttccctagcccccccagtgtgcaagggcagtgaagacttgattgtacaaaatacgttttgtaaatgttgtgctgttaacactgcaaataaacttggtagcaaacacttccaaaaaaaaaaaaaaaaaa

SEQ ID NO: 4 - DNA, 3699 bp, Homo sapiens
ttcattggaacagaaagaaatggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactgactgcagccagccacaggtacagagccacaggaccccaagaatgagcttacaaagtggcctttccaggccctgggagctcctctcactcttcagtccttctactgtcctggctactaaatattttatgtacatcagcctgaaaaggacttctggctatgcaagggtcccttaaagattttctgcttgaagtctcccttggaaatctgccatgagcacaaaattatggtaatttttcacctgagaagattttaaaaccatttaaacgccaccaattgagcaagatgctgattcattatttatcagccctattctttctattcaggctgttgttggcttagggctggaagcacagagtggcttggcctcaagagaatagctggtttccctaagtttacttctctaaaaccctgtgttcacaaaggcagagagtcagacccttcaatggaaggagagtgcttgggatcgattatgtgacttaaagtcagaatagtccttgggcagttctcaaatgttggagtggaacattggggaggaaattctgaggcaggtattagaaatgaaaaggaaacttgaaacctgggcatggtggctcacgcctgtaatcccagcactttgggaggccaaggtgggcagatcactggaggtcaggagttcgaaaccagcctggccaacatggtgaaaccccatctctactaaaaatacagaaattagccggtcatggtggtggacacctgtaatcccagctactcaggtggctaaggcaggagaatcacttcagcccgggaggtggaggttgcagtgagccaagatcataccacggcactccagcctgggtgacagtgagactgtggctcaaaaaaaaaaaaaaaaaaaggaaaatgaaactagaagagatttctaaaagtctgagatatatttgctagatttctaaagaatgtgttctaaaacagcagaagattttcaagaaccggtttccaaagacagtcttctaattcctcattagtaataagtaaaatgtttattgttgtagctctggtatataatccattcctcttaaaatataagacctctggcatgaatatttcatatctataaaatgacagatcccaccaggaaggaagctgttgctttctttgaggtgatttttttcctttgctccctgttgctgaaaccatacagcttcataaataattttgcttgctgaaggaagaaaaagtgtttttcataaacccattatccaggactgtttatagctgttggaaggactaggtcttccctagcccccccagtgtgcaagggcagtgaagacttgattgtacaaaatacgttttgtaaatgttgtgctgttaacactgcaaataaacttggtagcaaacacttccaaaaaaaaaaaaaaaaaa

SEQ ID NO: 5 - DNA, 3800 bp, Homo sapiens
cttagcggtagccccttggtttccgtggcaacggaaaagcgcgggaattacagataaattaaaactgcgactgcgcggcgtgagctcgctgagacttcctggacgggggacaggctgtggggtttctcagataactgggcccctgcgctcaggaggccttcaccctctgctctggttcattggaacagaaagaaatggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactgactgcagccagccacaggtacagagccacaggaccccaagaatgagcttacaaagtggcctttccaggccctgggagctcctctcactcttcagtccttctactgtcctggctactaaatattttatgtacatcagcctgaaaaggacttctggctatgcaagggtcccttaaagattttctgcttgaagtctcccttggaaatctgccatgagcacaaaattatggtaatttttcacctgagaagattttaaaaccatttaaacgccaccaattgagcaagatgctgattcattatttatcagccctattctttctattcaggctgttgttggcttagggctggaagcacagagtggcttggcctcaagagaatagctggtttccctaagtttacttctctaaaaccctgtgttcacaaaggcagagagtcagacccttcaatggaaggagagtgcttgggatcgattatgtgacttaaagtcagaatagtccttgggcagttctcaaatgttggagtggaacattggggaggaaattctgaggcaggtattagaaatgaaaaggaaacttgaaacctgggcatggtggctcacgcctgtaatcccagcactttgggaggccaaggtgggcagatcactggaggtcaggagttcgaaaccagcctggccaacatggtgaaaccccatctctactaaaaatacagaaattagccggtcatggtggtggacacctgtaatcccagctactcaggtggctaaggcaggagaatcacttcagcccgggaggtggaggttgcagtgagccaagatcataccacggcactccagcctgggtgacagtgagactgtggctcaaaaaaaaaaaaaaaaaaaggaaaatgaaactagaagagatttctaaaagtctgagatatatttgctagatttctaaagaatgtgttctaaaacagcagaagattttcaagaaccggtttccaaagacagtcttctaattcctcattagtaataagtaaaatgtttattgttgtagctctggtatataatccattcctcttaaaatataagacctctggcatgaatatttcatatctataaaatgacagatcccaccaggaaggaagctgttgctttctttgaggtgatttttttcctttgctccctgttgctgaaaccatacagcttcataaataattttgcttgctgaaggaagaaaaagtgtttttcataaacccattatccaggactgtttatagctgttggaaggactaggtcttccctagcccccccagtgtgcaagggcagtgaagacttgattgtacaaaatacgttttgtaaatgttgtgctgttaacactgcaaataaacttggtagcaaacacttccaaaaaaaaaaaaaaaaaa

SEQ ID NO: 6 - DNA, 11386 bp, Homo sapiens
gtggcgcgagcttctgaaactaggcggcagaggcggagccgctgtggcactgctgcgcctctgctgcgcctcgggtgtcttttgcggcggtgggtcgccgccgggagaagcgtgaggggacagatttgtgaccggcgcggtttttgtcagcttactccggccaaaaaagaactgcacctctggagcggacttatttaccaagcattggaggaatatcgtaggtaaaaatgcctattggatccaaagagaggccaacattttttgaaatttttaagacacgctgcaacaaagcagatttaggaccaataagtcttaattggtttgaagaactttcttcagaagctccaccctataattctgaacctgcagaagaatctgaacataaaaacaacaattacgaaccaaacctatttaaaactccacaaaggaaaccatcttataatcagctggcttcaactccaataatattcaaagagcaagggctgactctgccgctgtaccaatctcctgtaaaagaattagataaattcaaattagacttaggaaggaatgttcccaatagtagacataaaagtcttcgcacagtgaaaactaaaatggatcaagcagatgatgtttcctgtccacttctaaattcttgtcttagtgaaagtcctgttgttctacaatgtacacatgtaacaccacaaagagataagtcagtggtatgtgggagtttgtttcatacaccaaagtttgtgaagggtcgtcagacaccaaaacatatttctgaaagtctaggagctgaggtggatcctgatatgtcttggtcaagttctttagctacaccacccacccttagttctactgtgctcatagtcagaaatgaagaagcatctgaaactgtatttcctcatgatactactgctaatgtgaaaagctatttttccaatcatgatgaaagtctgaagaaaaatgatagatttatcgcttctgtgacagacagtgaaaacacaaatcaaagagaagctgcaagtcatggatttggaaaaacatcagggaattcatttaaagtaaatagctgcaaagaccacattggaaagtcaatgccaaatgtcctagaagatgaagtatatgaaacagttgtagatacctctgaagaagatagtttttcattatgtttttctaaatgtagaacaaaaaatctacaaaaagtaagaactagcaagactaggaaaaaaattttccatgaagcaaacgctgatgaatgtgaaaaatctaaaaaccaagtgaaagaaaaatactcatttgtatctgaagtggaaccaaatgatactgatccattagattcaaatgtagcaaatcagaagccctttgagagtggaagtgacaaaatctccaaggaagttgtaccgtctttggcctgtgaatggtctcaactaaccctttcaggtctaaatggagcccagatggagaaaatacccctattgcatatttcttcatgtgaccaaaatatttcagaaaaagacctattagacacagagaacaaaagaaagaaagattttcttacttcagagaattctttgccacgtatttctagcctaccaaaatcagagaagccattaaatgaggaaacagtggtaaataagagagatgaagagcagcatcttgaatctcatacagactgcattcttgcagtaaagcaggcaatatctggaacttctccagtggcttcttcatttcagggtatcaaaaagtctatattcagaataagagaatcacctaaagagactttcaatgcaagtttttcaggtcatatgactgatccaaactttaaaaaagaaactgaagcctctgaaagtggactggaaatacatactgtttgctcacagaaggaggactccttatgtccaaatttaattgataatggaagctggccagccaccaccacacagaattctgtagctttgaagaatgcaggtttaatatccactttgaaaaagaaaacaaataagtttatttatgctatacatgatgaaacatcttataaaggaaaaaaaataccgaaagaccaaaaatcagaactaattaactgttcagcccagtttgaagcaaatgcttttgaagcaccacttacatttgcaaatgctgattcaggtttattgcattcttctgtgaaaagaagctgttcacagaatgattctgaagaaccaactttgtccttaactagctcttttgggacaattctgaggaaatgttctagaaatgaaacatgttctaataatacagtaatctctcaggatcttgattataaagaagcaaaatgtaataaggaaaaactacagttatttattaccccagaagctgattctctgtcatgcctgcaggaaggacagtgtgaaaatgatccaaaaagcaaaaaagtttcagatataaaagaagaggtcttggctgcagcatgtcacccagtacaacattcaaaagtggaatacagtgatactgactttcaatcccagaaaagtcttttatatgatcatgaaaatgccagcactcttattttaactcctacttccaaggatgttctgtcaaacctagtcatgatttctagaggcaaagaatcatacaaaatgtcagacaagctcaaaggtaacaattatgaatctgatgttgaattaaccaaaaatattcccatggaaaagaatcaagatgtatgtgctttaaatgaaaattataaaaacgttgagctgttgccacctgaaaaatacatgagagtagcatcaccttcaagaaaggtacaattcaaccaaaacacaaatctaagagtaatccaaaaaaatcaagaagaaactacttcaatttcaaaaataactgtcaatccagactctgaagaacttttctcagacaatgagaataattttgtcttccaagtagctaatgaaaggaataatcttgctttaggaaatactaaggaacttcatgaaacagacttgacttgtgtaaacgaacccattttcaagaactctaccatggttttatatggagacacaggtgataaacaagcaacccaagtgtcaattaaaaaagatttggtttatgttcttgcagaggagaacaaaaatagtgtaaagcagcatataaaaatgactctaggtcaagatttaaaatcggacatctccttgaatatagataaaataccagaaaaaaataatgattacatgaacaaatgggcaggactcttaggtccaatttcaaatcacagttttggaggtagcttcagaacagcttcaaataaggaaatcaagctctctgaacataacattaagaagagcaaaatgttcttcaaagatattgaagaacaatatcctactagtttagcttgtgttgaaattgtaaataccttggcattagataatcaaaagaaactgagcaagcctcagtcaattaatactgtatctgcacatttacagagtagtgtagttgtttctgattgtaaaaatagtcatataacccctcagatgttattttccaagcaggattttaattcaaaccataatttaacacctagccaaaaggcagaaattacagaactttctactatattagaagaatcaggaagtcagtttgaatttactcagtttagaaaaccaagctacatattgcagaagagtacatttgaagtgcctgaaaaccagatgactatcttaaagaccacttctgaggaatgcagagatgctgatcttcatgtcataatgaatgccccatcgattggtcaggtagacagcagcaagcaatttgaaggtacagttgaaattaaacggaagtttgctggcctgttgaaaaatgactgtaacaaaagtgcttctggttatttaacagatgaaaatgaagtggggtttaggggcttttattctgctcatggcacaaaactgaatgtttctactgaagctctgcaaaaagctgtgaaactgtttagtgatattgagaatattagtgaggaaacttctgcagaggtacatccaataagtttatcttcaagtaaatgtcatgattctgttgtttcaatgtttaagatagaaaatcataatgataaaactgtaagtgaaaaaaataataaatgccaactgatattacaaaataatattgaaatgactactggcacttttgttgaagaaattactgaaaattacaagagaaatactgaaaatgaagataacaaatatactgctgccagtagaaattctcataacttagaatttgatggcagtgattcaagtaaaaatgatactgtttgtattcataaagatgaaacggacttgctatttactgatcagcacaacatatgtcttaaattatctggccagtttatgaaggagggaaacactcagattaaagaagatttgtcagatttaacttttttggaagttgcgaaagctcaagaagcatgtcatggtaatacttcaaataaagaacagttaactgctactaaaacggagcaaaatataaaagattttgagacttctgatacattttttcagactgcaagtgggaaaaatattagtgtcgccaaagagtcatttaataaaattgtaaatttctttgatcagaaaccagaagaattgcataacttttccttaaattctgaattacattctgacataagaaagaacaaaatggacattctaagttatgaggaaacagacatagttaaacacaaaatactgaaagaaagtgtcccagttggtactggaaatcaactagtgaccttccagggacaacccgaacgtgatgaaaagatcaaagaacctactctattgggttttcatacagctagcgggaaaaaagttaaaattgcaaaggaatctttggacaaagtgaaaaacctttttgatgaaaaagagcaaggtactagtgaaatcaccagttttagccatcaatgggcaaagaccctaaagtacagagaggcctgtaaagaccttgaattagcatgtgagaccattgagatcacagctgccccaaagtgtaaagaaatgcagaattctctcaataatgataaaaaccttgtttctattgagactgtggtgccacctaagctcttaagtgataatttatgtagacaaactgaaaatctcaaaacatcaaaaagtatctttttgaaagttaaagtacatgaaaatgtagaaaaagaaacagcaaaaagtcctgcaacttgttacacaaatcagtccccttattcagtcattgaaaattcagccttagctttttacacaagttgtagtagaaaaacttctgtgagtcagacttcattacttgaagcaaaaaaatggcttagagaaggaatatttgatggtcaaccagaaagaataaatactgcagattatgtaggaaattatttgtatgaaaataattcaaacagtactatagctgaaaatgacaaaaatcatctctccgaaaaacaagatacttatttaagtaacagtagcatgtctaacagctattcctaccattctgatgaggtatataatgattcaggatatctctcaaaaaataaacttgattctggtattgagccagtattgaagaatgttgaagatcaaaaaaacactagtttttccaaagtaatatccaatgtaaaagatgcaaatgcatacccacaaactgtaaatgaagatatttgcgttgaggaacttgtgactagctcttcaccctgcaaaaataaaaatgcagccattaaattgtccatatctaatagtaataattttgaggtagggccacctgcatttaggatagccagtggtaaaatcgtttgtgtttcacatgaaacaattaaaaaagtgaaagacatatttacagacagtttcagtaaagtaattaaggaaaacaacgagaataaatcaaaaatttgccaaacgaaaattatggcaggttgttacgaggcattggatgattcagaggatattcttcataactctctagataatgatgaatgtagcacgcattcacataaggtttttgctgacattcagagtgaagaaattttacaacataaccaaaatatgtctggattggagaaagtttctaaaatatcaccttgtgatgttagtttggaaacttcagatatatgtaaatgtagtatagggaagcttcataagtcagtctcatctgcaaatacttgtgggatttttagcacagcaagtggaaaatctgtccaggtatcagatgcttcattacaaaacgcaagacaagtgttttctgaaatagaagatagtaccaagcaagtcttttccaaagtattgtttaaaagtaacgaacattcagaccagctcacaagagaagaaaatactgctatacgtactccagaacatttaatatcccaaaaaggcttttcatataatgtggtaaattcatctgctttctctggatttagtacagcaagtggaaagcaagtttccattttagaaagttccttacacaaagttaagggagtgttagaggaatttgatttaatcagaactgagcatagtcttcactattcacctacgtctagacaaaatgtatcaaaaatacttcctcgtgttgataagagaaacccagagcactgtgtaaactcagaaatggaaaaaacctgcagtaaagaatttaaattatcaaataacttaaatgttgaaggtggttcttcagaaaataatcactctattaaagtttctccatatctctctcaatttcaacaagacaaacaacagttggtattaggaaccaaagtgtcacttgttgagaacattcatgttttgggaaaagaacaggcttcacctaaaaacgtaaaaatggaaattggtaaaactgaaactttttctgatgttcctgtgaaaacaaatatagaagtttgttctacttactccaaagattcagaaaactactttgaaacagaagcagtagaaattgctaaagcttttatggaagatgatgaactgacagattctaaactgccaagtcatgccacacattctctttttacatgtcccgaaaatgaggaaatggttttgtcaaattcaagaattggaaaaagaagaggagagccccttatcttagtgggagaaccctcaatcaaaagaaacttattaaatgaatttgacaggataatagaaaatcaagaaaaatccttaaaggcttcaaaaagcactccagatggcacaataaaagatcgaagattgtttatgcatcatgtttctttagagccgattacctgtgtaccctttcgcacaactaaggaacgtcaagagatacagaatccaaattttaccgcacctggtcaagaatttctgtctaaatctcatttgtatgaacatctgactttggaaaaatcttcaagcaatttagcagtttcaggacatccattttatcaagtttctgctacaagaaatgaaaaaatgagacacttgattactacaggcagaccaaccaaagtctttgttccaccttttaaaactaaatcacattttcacagagttgaacagtgtgttaggaatattaacttggaggaaaacagacaaaagcaaaacattgatggacatggctctgatgatagtaaaaataagattaatgacaatgagattcatcagtttaacaaaaacaactccaatcaagcagcagctgtaactttcacaaagtgtgaagaagaacctttagatttaattacaagtcttcagaatgccagagatatacaggatatgcgaattaagaagaaacaaaggcaacgcgtctttccacagccaggcagtctgtatcttgcaaaaacatccactctgcctcgaatctctctgaaagcagcagtaggaggccaagttccctctgcgtgttctcataaacagctgtatacgtatggcgtttctaaacattgcataaaaattaacagcaaaaatgcagagtcttttcagtttcacactgaagattattttggtaaggaaagtttatggactggaaaaggaatacagttggctgatggtggatggctcataccctccaatgatggaaaggctggaaaagaagaattttatagggctctgtgtgacactccaggtgtggatccaaagcttatttctagaatttgggtttataatcactatagatggatcatatggaaactggcagctatggaatgtgcctttcctaaggaatttgctaatagatgcctaagcccagaaagggtgcttcttcaactaaaatacagatatgatacggaaattgatagaagcagaagatcggctataaaaaagataatggaaagggatgacacagctgcaaaaacacttgttctctgtgtttctgacataatttcattgagcgcaaatatatctgaaacttctagcaataaaactagtagtgcagatacccaaaaagtggccattattgaacttacagatgggtggtatgctgttaaggcccagttagatcctcccctcttagctgtcttaaagaatggcagactgacagttggtcagaagattattcttcatggagcagaactggtgggctctcctgatgcctgtacacctcttgaagccccagaatctcttatgttaaagatttctgctaacagtactcggcctgctcgctggtataccaaacttggattctttcctgaccctagaccttttcctctgcccttatcatcgcttttcagtgatggaggaaatgttggttgtgttgatgtaattattcaaagagcataccctatacagtggatggagaagacatcatctggattatacatatttcgcaatgaaagagaggaagaaaaggaagcagcaaaatatgtggaggcccaacaaaagagactagaagccttattcactaaaattcaggaggaatttgaagaacatgaagaaaacacaacaaaaccatatttaccatcacgtgcactaacaagacagcaagttcgtgctttgcaagatggtgcagagctttatgaagcagtgaagaatgcagcagacccagcttaccttgagggttatttcagtgaagagcagttaagagccttgaataatcacaggcaaatgttgaatgataagaaacaagctcagatccagttggaaattaggaaggccatggaatctgctgaacaaaaggaacaaggtttatcaagggatgtcacaaccgtgtggaagttgcgtattgtaagctattcaaaaaaagaaaaagattcagttatactgagtatttggcgtccatcatcagatttatattctctgttaacagaaggaaagagatacagaatttatcatcttgcaacttcaaaatctaaaagtaaatctgaaagagctaacatacagttagcagcgacaaaaaaaactcagtatcaacaactaccggtttcagatgaaattttatttcagatttaccagccacgggagccccttcacttcagcaaatttttagatccagactttcagccatcttgttctgaggtggacctaataggatttgtcgtttctgttgtgaaaaaaacaggacttgcccctttcgtctatttgtcagacgaatgttacaatttactggcaataaagttttggatagaccttaatgaggacattattaagcctcatatgttaattgctgcaagcaacctccagtggcgaccagaatccaaatcaggccttcttactttatttgctggagatttttctgtgttttctgctagtccaaaagagggccactttcaagagacattcaacaaaatgaaaaatactgttgagaatattgacatactttgcaatgaagcagaaaacaagcttatgcatatactgcatgcaaatgatcccaagtggtccaccccaactaaagactgtacttcagggccgtacactgctcaaatcattcctggtacaggaaacaagcttctgatgtcttctcctaattgtgagatatattatcaaagtcctttatcactttgtatggccaaaaggaagtctgtttccacacctgtctcagcccagatgacttcaaagtcttgtaaaggggagaaagagattgatgaccaaaagaactgcaaaaagagaagagccttggatttcttgagtagactgcctttacctccacctgttagtcccatttgtacatttgtttctccggctgcacagaaggcatttcagccaccaaggagttgtggcaccaaatacgaaacacccataaagaaaaaagaactgaattctcctcagatgactccatttaaaaaattcaatgaaatttctcttttggaaagtaattcaatagctgacgaagaacttgcattgataaatacccaagctcttttgtctggttcaacaggagaaaaacaatttatatctgtcagtgaatccactaggactgctcccaccagttcagaagattatctcagactgaaacgacgttgtactacatctctgatcaaagaacaggagagttcccaggccagtacggaagaatgtgagaaaaataagcaggacacaattacaactaaaaaatatatctaagcatttgcaaaggcgacaataaattattgacgcttaacctttccagtttataagactggaatataatttcaaaccacacattagtacttatgttgcacaatgagaaaagaaattagtttcaaatttacctcagcgtttgtgtatcgggcaaaaatcgttttgcccgattccgtattggtatacttttgcttcagttgcatatcttaaaactaaatgtaatttattaactaatcaagaaaaacatctttggctgagctcggtggctcatgcctgtaatcccaacactttgagaagctgaggtgggaggagtgcttgaggccaggagttcaagaccagcctgggcaacatagggagacccccatctttacaaagaaaaaaaaaaggggaaaagaaaatcttttaaatctttggatttgatcactacaagtattattttacaagtgaaataaacataccattttcttttagattgtgtcattaaatggaatgaggtctcttagtacagttattttgatgcagataattccttttagtttagctactattttaggggattttttttagaggtaactcactatgaaatagttctccttaatgcaaatatgttggttctgctatagttccatcctgttcaaaagtcaggatgaatatgaagagtggtgtttccttttgagcaattcttcatccttaagtcagcatgattataagaaaaatagaaccctcagtgtaactctaattcctttttactattccagtgtgatctctgaaattaaattacttcaactaaaaattcaaatactttaaatcagaagatttcatagttaatttattttttttttcaacaaaatggtcatccaaactcaaacttgagaaaatatcttgctttcaaattggcactgatt

SEQ ID NO: 7 - DNA, 4174 bp, Homo sapiens
cttttaaatttgcgttgtaagatttattttggctctccccgcctgttctttgcacattaaaaatgaaaaagtttgtagaactaagctaagcagatggtcttcctgcaaaaagaccgggctgaagtaaagcattgttttggagctggttcacagaaaaaaggcaaaactggttatcctgacttcaagctccaacataaactgctcgctttctccgggaaacttgccccgccacacacacttgactgcgtggccagttctttcgaagcctctcgctcccaacacggagttcctcccatttcttcacagtcggctctcagcagctgctgctggtttctcggctccagcaccacgagtaccgcactctgaggtttacaaagcactctgcttcaccgactgtgatcctcacagtcctgtccggtggcctcacgcaggtggcggtgcagcctttcaggcccagagcggccaggagcgaagcccgcagccccgcctggaagcgcagcgcggtcggtcgcgcgcccctgaggcttggaggcctgggcttcccccagcagcgctcgagcaccgcccagtcgagcctcacaccggatgccacttcatatttgggcccagagctcaattcgcgccgatgcggtccgccgtccttaaatctcttcagccaggatctctccccgactgcaaagcagccctgggcgggagcggcaacatctccacgtcacccttttggagccgccgacattcagaggggcaggacacgggaacgcgcgctgtcttgctttacggcgcgggtgcgcgagtttgcggcagcgtgacgccctcaagttttggcgggaaaagcgctgcatttggattcctgcagtggtgggcaaaggacagtccgccgaggtgctcggtggagtcatggcagtgccctttgtggaagactgggacttggtgcaaaccctgggagaaggtgcctatggagaagttcaacttgctgtgaatagagtaactgaagaagcagtcgcagtgaagattgtagatatgaagcgtgccgtagactgtccagaaaatattaagaaagagatctgtatcaataaaatgctaaatcatgaaaatgtagtaaaattctatggtcacaggagagaaggcaatatccaatatttatttctggagtactgtagtggaggagagctttttgacagaatagagccagacataggcatgcctgaaccagatgctcagagattcttccatcaactcatggcaggggtggtttatctgcatggtattggaataactcacagggatattaaaccagaaaatcttctgttggatgaaagggataacctcaaaatctcagactttggcttggcaacagtatttcggtataataatcgtgagcgtttgttgaacaagatgtgtggtactttaccatatgttgctccagaacttctgaagagaagagaatttcatgcagaaccagttgatgtttggtcctgtggaatagtacttactgcaatgctcgctggagaattgccatgggaccaacccagtgacagctgtcaggagtattctgactggaaagaaaaaaaaacatacctcaacccttggaaaaaaatcgattctgctcctctagctctgctgcataaaatcttagttgagaatccatcagcaagaattaccattccagacatcaaaaaagatagatggtacaacaaacccctcaagaaaggggcaaaaaggccccgagtcacttcaggtggtgtgtcagagtctcccagtggattttctaagcacattcaatccaatttggacttctctccagtaaacagtgcttctagtgaagaaaatgtgaagtactccagttctcagccagaaccccgcacaggtctttccttatgggataccagcccctcatacattgataaattggtacaagggatcagcttttcccagcccacatgtcctgatcatatgcttttgaatagtcagttacttggcaccccaggatcctcacagaacccctggcagcggttggtcaaaagaatgacacgattctttaccaaattggatgcagacaaatcttatcaatgcctgaaagagacttgtgagaagttgggctatcaatggaagaaaagttgtatgaatcaggttactatatcaacaactgataggagaaacaataaactcattttcaaagtgaatttgttagaaatggatgataaaatattggttgacttccggctttctaagggtgatggattggagttcaagagacacttcctgaagattaaagggaagctgattgatattgtgagcagccagaagatttggcttcctgccacatgatcggaccatcggctctggggaatcctggtgaatatagtgctgctatgttgacattattcttcctagagaagattatcctgtcctgcaaactgcaaatagtagttcctgaagtgttcacttccctgtttatccaaacatcttccaatttattttgtttgttcggcatacaaataatacctatatcttaattgtaagcaaaactttggggaaaggatgaatagaattcatttgattatttcttcatgtgtgtttagtatctgaatttgaaactcatctggtggaaaccaagtttcaggggacatgagttttccagcttttatacacacgtatctcatttttatcaaaacattttgtttaattcaaaaagtacatattccatgttgatttaattctaagatgaaccaataaagacataattcttgtgacttttggacagtagatttatcagtctgtgaagcgaagccagcttcaaaacatatccccaagatttgtacttatattttcaaaagggcctggccagttatataaacctgtttttgaattataatgattaattaaaattgcaagtaggtgttttttccagtgtagttagtaaaatacttgtattttacagtgttgcataaactctagtgcttaactaactttactctaaaaattactgttgaacatcttaaatatttttctatattttctactttcatagccatattttaaccttttcaacttactggtgaccaagcttttaggtgataaagaataaaagagggaagggaagagtaaggaagctataagaaaaatagatctgattctttgttcctttacctgttagacttacaaaaagtttgtttttctaataaaatttgtatcaactttggggcatattaggttgaggccttggctcctgcctgtagtcccagctacttaggaggctgagagaggaggatcgcgtgaacctggaagtttgaggctgtagtgagctatgattgcaccagtgcactccagcttggatgacagagtaagaccctacctctaataaaaatttttaaaattgtaaaacattataaaattaatcagttattttaatctgaagccaagaacatgtagaatgttatgattagagtttatcacatattaatgtatactggcaaattgtgttactggagtatacccataggaggaataaattcaaacctgttttatttatttgaacctatttacggtatgcttaagaattgaatcagtataaattctcaaatatgggagaaattttgttcttgagaattatctgagtcattaatatttttcaaaaacagctctcactgacttgaacctcttctgtaagctctaaccttttacctgctttacatttccacttgaatgtctagtaggcatctcttgaccaaaaacagcttttgattcctgttctccaacctgttcctctcctagttttctccatctcagaaatgttacttcctctgcaaagtctttccctgacttatctaaaataataacctcctctgtttgctgtgggaatttgtatagaatggtgggaaaatttcaagtttcatatttggattagctctgacatttatttatctgaacactggtaattgcctcagtaaagacactgataataagtaccttttagagttattttaatctttaatgctttaatgtgtaggaagagtatagtgtcctgttttgcacagaaaggcattctgtaaataataagttgccttaattttcctgtaatgttcattatattgttgtgggaaggtatttactcctattattaaaaataaaaatgtgtaaaatttactacctgaaaaaaaaaaaaaaaaaaaaa

SEQ ID NO: 8 - DNA, 4072 bp, Homo sapiens
cttttaaatttgcgttgtaagatttattttggctctccccgcctgttctttgcacattaaaaatgaaaaagtttgtagaactaagctaagcagatggtcttcctgcaaaaagaccgggctgaagtaaagcattgttttggagctggttcacagaaaaaaggcaaaactggttatcctgacttcaagctccaacataaactgctcgctttctccgggaaacttgccccgccacacacacttgactgcgtggccagttctttcgaagcctctcgctcccaacacggagttcctcccatttcttcacagtcggctctcagcagctgctgctggtttctcggctccagcaccacgagtaccgcactctgaggtttacaaagcactctgcttcaccgactgtgatcctcacagtcctgtccggtggcctcacgcaggtggcggtgcagcctttcaggcccagagcggccaggagcgaagcccgcagccccgcctggaagcgcagcgcggtcggtcgcgcgcccctgaggcttggaggcctgggcttcccccagcagcgctcgagcaccgcccagtcgagcctcacaccggatgccacttcatatttgggcccagagctcaattcgcgccgatgcggtccgccgtccttaaatctcttcagccaggatctctccccgactgcaaagcagccctgggcgggagcggcaacatctccacgtcacccttttggagccgccgacattcagaggggcaggacacgggaacgcgcgctgtcttgctttacggcgcgggtgcgcgagtttgcggcagcgtgacgccctcaagttttggcgggaaaagcgctgcatttggattcctgcagtggtgggcaaaggacagtccgccgaggtgctcggtggagtcatggcagtgccctttgtggaagactgggacttggtgcaaaccctgggagaaggtgcctatggagaagttcaacttgctgtgaatagagtaactgaagaagcagtcgcagtgaagattgtagatatgaagcgtgccgtagactgtccagaaaatattaagaaagagatctgtatcaataaaatgctaaatcatgaaaatgtagtaaaattctatggtcacaggagagaaggcaatatccaatatttatttctggagtactgtagtggaggagagctttttgacagaatagagccagacataggcatgcctgaaccagatgctcagagattcttccatcaactcatggcaggggtggtttatctgcatggtattggaataactcacagggatattaaaccagaaaatcttctgttggatgaaagggataacctcaaaatctcagactttggcttggcaacagtatttcggtataataatcgtgagcgtttgttgaacaagatgtgtggtactttaccatatgttgctccagaacttctgaagagaagagaatttcatgcagaaccagttgatgtttggtcctgtggaatagtacttactgcaatgctcgctggagaattgccatgggaccaacccagtgacagctgtcaggagtattctgactggaaagaaaaaaaaacatacctcaacccttggaaaaaaatcgattctgctcctctagctctgctgcataaaatcttagttgagaatccatcagcaagaattaccattccagacatcaaaaaagatagatggtacaacaaacccctcaagaaaggggcaaaaaggccccgagtcacttcaggtggtgtgtcagagtctcccagtggattttctaagcacattcaatccaatttggacttctctccagtaaacagtgcttctagtgaagaaaatgtgaagtactccagttctcagccagaaccccgcacaggtctttccttatgggataccagcccctcatacattgataaattggtacaagggatcagcttttcccagcccacatgtcctgatcatatgcttttgaatagtcagttacttggcaccccaggatcctcacagaacccctggcagcggttggtcaaaagaatgacacgattctttaccaaattggatgcagacaaatcttatcaatgcctgaaagagacttgtgagaagttgggctatcaatggaagaaaagttgtatgaatcagggtgatggattggagttcaagagacacttcctgaagattaaagggaagctgattgatattgtgagcagccagaagatttggcttcctgccacatgatcggaccatcggctctggggaatcctggtgaatatagtgctgctatgttgacattattcttcctagagaagattatcctgtcctgcaaactgcaaatagtagttcctgaagtgttcacttccctgtttatccaaacatcttccaatttattttgtttgttcggcatacaaataatacctatatcttaattgtaagcaaaactttggggaaaggatgaatagaattcatttgattatttcttcatgtgtgtttagtatctgaatttgaaactcatctggtggaaaccaagtttcaggggacatgagttttccagcttttatacacacgtatctcatttttatcaaaacattttgtttaattcaaaaagtacatattccatgttgatttaattctaagatgaaccaataaagacataattcttgtgacttttggacagtagatttatcagtctgtgaagcgaagccagcttcaaaacatatccccaagatttgtacttatattttcaaaagggcctggccagttatataaacctgtttttgaattataatgattaattaaaattgcaagtaggtgttttttccagtgtagttagtaaaatacttgtattttacagtgttgcataaactctagtgcttaactaactttactctaaaaattactgttgaacatcttaaatatttttctatattttctactttcatagccatattttaaccttttcaacttactggtgaccaagcttttaggtgataaagaataaaagagggaagggaagagtaaggaagctataagaaaaatagatctgattctttgttcctttacctgttagacttacaaaaagtttgtttttctaataaaatttgtatcaactttggggcatattaggttgaggccttggctcctgcctgtagtcccagctacttaggaggctgagagaggaggatcgcgtgaacctggaagtttgaggctgtagtgagctatgattgcaccagtgcactccagcttggatgacagagtaagaccctacctctaataaaaatttttaaaattgtaaaacattataaaattaatcagttattttaatctgaagccaagaacatgtagaatgttatgattagagtttatcacatattaatgtatactggcaaattgtgttactggagtatacccataggaggaataaattcaaacctgttttatttatttgaacctatttacggtatgcttaagaattgaatcagtataaattctcaaatatgggagaaattttgttcttgagaattatctgagtcattaatatttttcaaaaacagctctcactgacttgaacctcttctgtaagctctaaccttttacctgctttacatttccacttgaatgtctagtaggcatctcttgaccaaaaacagcttttgattcctgttctccaacctgttcctctcctagttttctccatctcagaaatgttacttcctctgcaaagtctttccctgacttatctaaaataataacctcctctgtttgctgtgggaatttgtatagaatggtgggaaaatttcaagtttcatatttggattagctctgacatttatttatctgaacactggtaattgcctcagtaaagacactgataataagtaccttttagagttattttaatctttaatgctttaatgtgtaggaagagtatagtgtcctgttttgcacagaaaggcattctgtaaataataagttgccttaattttcctgtaatgttcattatattgttgtgggaaggtatttactcctattattaaaaataaaaatgtgtaaaatttactacctgaaaaaaaaaaaaaaaaaaaaa

SEQ ID NO: 9 - DNA, 1991 bp, Homo sapiens
gcaggtttagcgccactctgctggctgaggctgcggagagtgtgcggctccaggtgggctcacgcggtcgtgatgtctcgggagtcggatgttgaggctcagcagtctcatggcagcagtgcctgttcacagccccatggcagcgttacccagtcccaaggctcctcctcacagtcccagggcatatccagctcctctaccagcacgatgccaaactccagccagtcctctcactccagctctgggacactgagctccttagagacagtgtccactcaggaactctattctattcctgaggaccaagaacctgaggaccaagaacctgaggagcctacccctgccccctgggctcgattatgggcccttcaggatggatttgccaatcttgagacagagtctggccatgttacccaatctgatcttgaactcctgctgtcatctgatcctcctgcctcagcctcccaaagtgctgggataagaggtgtgaggcaccatccccggccagtttgcagtctaaaatgtgtgaatgacaactactggtttgggagggacaaaagctgtgaatattgctttgatgaaccactgctgaaaagaacagataaataccgaacatacagcaagaaacactttcggattttcagggaagtgggtcctaaaaactcttacattgcatacatagaagatcacagtggcaatggaacctttgtaaatacagagcttgtagggaaaggaaaacgccgtcctttgaataacaattctgaaattgcactgtcactaagcagaaataaagtttttgtcttttttgatctgactgtagatgatcagtcagtttatcctaaggcattaagagatgaatacatcatgtcaaaaactcttggaagtggtgcctgtggagaggtaaagctggctttcgagaggaaaacatgtaagaaagtagccataaagatcatcagcaaaaggaagtttgctattggttcagcaagagaggcagacccagctctcaatgttgaaacagaaatagaaattttgaaaaagctaaatcatccttgcatcatcaagattaaaaacttttttgatgcagaagattattatattgttttggaattgatggaagggggagagctgtttgacaaagtggtggggaataaacgcctgaaagaagctacctgcaagctctatttttaccagatgctcttggctgtgcagtaccttcatgaaaacggtattatacaccgtgacttaaagccagagaatgttttactgtcatctcaagaagaggactgtcttataaagattactgattttgggcactccaagattttgggagagacctctctcatgagaaccttatgtggaacccccacctacttggcgcctgaagttcttgtttctgttgggactgctgggtataaccgtgctgtggactgctggagtttaggagttattctttttatctgccttagtgggtatccacctttctctgagcataggactcaagtgtcactgaaggatcagatcaccagtggaaaatacaacttcattcctgaagtctgggcagaagtctcagagaaagctctggaccttgtcaagaagttgttggtagtggatccaaaggcacgttttacgacagaagaagccttaagacacccgtggcttcaggatgaagacatgaagagaaagtttcaagatcttctgtctgaggaaaatgaatccacagctctaccccaggttctagcccagccttctactagtcgaaagcggccccgtgaaggggaagccgagggtgccgagaccacaaagcgcccagctgtgtgtgctgctgtgttgtgaactccgtggtttgaacacgaaagaaatgtaccttctttcactctgtcatctttcttttctttgagtctgtttttttatagtttgtattttaattatgggaataattgctttttcacagtcactgatgtacaattaaaaacctgatggaacctggaaaa

SEQ ID NO: 10 - DNA, 1862 bp, Homo sapiens
gcaggtttagcgccactctgctggctgaggctgcggagagtgtgcggctccaggtgggctcacgcggtcgtgatgtctcgggagtcggatgttgaggctcagcagtctcatggcagcagtgcctgttcacagccccatggcagcgttacccagtcccaaggctcctcctcacagtcccagggcatatccagctcctctaccagcacgatgccaaactccagccagtcctctcactccagctctgggacactgagctccttagagacagtgtccactcaggaactctattctattcctgaggaccaagaacctgaggaccaagaacctgaggagcctacccctgccccctgggctcgattatgggcccttcaggatggatttgccaatcttgaatgtgtgaatgacaactactggtttgggagggacaaaagctgtgaatattgctttgatgaaccactgctgaaaagaacagataaataccgaacatacagcaagaaacactttcggattttcagggaagtgggtcctaaaaactcttacattgcatacatagaagatcacagtggcaatggaacctttgtaaatacagagcttgtagggaaaggaaaacgccgtcctttgaataacaattctgaaattgcactgtcactaagcagaaataaagtttttgtcttttttgatctgactgtagatgatcagtcagtttatcctaaggcattaagagatgaatacatcatgtcaaaaactcttggaagtggtgcctgtggagaggtaaagctggctttcgagaggaaaacatgtaagaaagtagccataaagatcatcagcaaaaggaagtttgctattggttcagcaagagaggcagacccagctctcaatgttgaaacagaaatagaaattttgaaaaagctaaatcatccttgcatcatcaagattaaaaacttttttgatgcagaagattattatattgttttggaattgatggaagggggagagctgtttgacaaagtggtggggaataaacgcctgaaagaagctacctgcaagctctatttttaccagatgctcttggctgtgcagtaccttcatgaaaacggtattatacaccgtgacttaaagccagagaatgttttactgtcatctcaagaagaggactgtcttataaagattactgattttgggcactccaagattttgggagagacctctctcatgagaaccttatgtggaacccccacctacttggcgcctgaagttcttgtttctgttgggactgctgggtataaccgtgctgtggactgctggagtttaggagttattctttttatctgccttagtgggtatccacctttctctgagcataggactcaagtgtcactgaaggatcagatcaccagtggaaaatacaacttcattcctgaagtctgggcagaagtctcagagaaagctctggaccttgtcaagaagttgttggtagtggatccaaaggcacgttttacgacagaagaagccttaagacacccgtggcttcaggatgaagacatgaagagaaagtttcaagatcttctgtctgaggaaaatgaatccacagctctaccccaggttctagcccagccttctactagtcgaaagcggccccgtgaaggggaagccgagggtgccgagaccacaaagcgcccagctgtgtgtgctgctgtgttgtgaactccgtggtttgaacacgaaagaaatgtaccttctttcactctgtcatctttcttttctttgagtctgtttttttatagtttgtattttaattatgggaataattgctttttcacagtcactgatgtacaattaaaaacctgatggaacctggaaaa

SEQ ID NO: 11 - DNA, 1775 bp, Homo sapiens
gcaggtttagcgccactctgctggctgaggctgcggagagtgtgcggctccaggtgggctcacgcggtcgtgatgtctcgggagtcggatgttgaggctcagcagtctcatggcagcagtgcctgttcacagccccatggcagcgttacccagtcccaaggctcctcctcacagtcccagggcatatccagctcctctaccagcacgatgccaaactccagccagtcctctcactccagctctgggacactgagctccttagagacagtgtccactcaggaactctattctattcctgaggaccaagaacctgaggaccaagaacctgaggagcctacccctgccccctgggctcgattatgggcccttcaggatggatttgccaatcttgaatgtgtgaatgacaactactggtttgggagggacaaaagctgtgaatattgctttgatgaaccactgctgaaaagaacagataaataccgaacatacagcaagaaacactttcggattttcagggaagtgggtcctaaaaactcttacattgcatacatagaagatcacagtggcaatggaacctttgtaaatacagagcttgtagggaaaggaaaacgccgtcctttgaataacaattctgaaattgcactgtcactaagcagaaataaagtttttgtcttttttgatctgactgtagatgatcagtcagtttatcctaaggcattaagagatgaatacatcatgtcaaaaactcttggaagtggtgcctgtggagaggtaaagctggctttcgagaggaaaacatgtaagaaagtagccataaagatcatcagcaaaaggaagtttgctattggttcagcaagagaggcagacccagctctcaatgttgaaacagaaatagaaattttgaaaaagctaaatcatccttgcatcatcaagattaaaaacttttttgatgcagaagattattatattgttttggaattgatggaagggggagagctgtttgacaaagtggtggggaataaacgcctgaaagaagctacctgcaagctctatttttaccagatgctcttggctgtgcagattactgattttgggcactccaagattttgggagagacctctctcatgagaaccttatgtggaacccccacctacttggcgcctgaagttcttgtttctgttgggactgctgggtataaccgtgctgtggactgctggagtttaggagttattctttttatctgccttagtgggtatccacctttctctgagcataggactcaagtgtcactgaaggatcagatcaccagtggaaaatacaacttcattcctgaagtctgggcagaagtctcagagaaagctctggaccttgtcaagaagttgttggtagtggatccaaaggcacgttttacgacagaagaagccttaagacacccgtggcttcaggatgaagacatgaagagaaagtttcaagatcttctgtctgaggaaaatgaatccacagctctaccccaggttctagcccagccttctactagtcgaaagcggccccgtgaaggggaagccgagggtgccgagaccacaaagcgcccagctgtgtgtgctgctgtgttgtgaactccgtggtttgaacacgaaagaaatgtaccttctttcactctgtcatctttcttttctttgagtctgtttttttatagtttgtattttaattatgggaataattgctttttcacagtcactgatgtacaattaaaaacctgatggaacctggaaaa

SEQ ID NO: 12 - DNA, 5164 bp, Homo sapiens
acgttatccatgaagtgtcgcgagagaaacggacgccgttctctcccgcggaattcaggtttacggccctgcgggttctcagaggcaagttcagaccgtgttgttttcttttcacggatcctgccctttcttcccgaaaagaagacagccttgggtcgcgattgtggggcttcgaagagtccagcagtgggaatttctagaatttggaatcgagtgcattttctgacatttgagtacagtacccaggggttcttggagaagaacctggtcccagaggagcttgactgaccataaaaatgagtactgcagatgcacttgatgatgaaaacacatttaaaatattagttgcaacagatattcatcttggatttatggagaaagatgcagtcagaggaaatgatacgtttgtaacactcgatgaaattttaagacttgcccaggaaaatgaagtggattttattttgttaggtggtgatctttttcatgaaaataagccctcaaggaaaacattacatacctgcctcgagttattaagaaaatattgtatgggtgatcggcctgtccagtttgaaattctcagtgatcagtcagtcaactttggttttagtaagtttccatgggtgaactatcaagatggcaacctcaacatttcaattccagtgtttagtattcatggcaatcatgacgatcccacaggggcagatgcactttgtgccttggacattttaagttgtgctggatttgtaaatcactttggacgttcaatgtctgtggagaagatagacattagtccggttttgcttcaaaaaggaagcacaaagattgcgctatatggtttaggatccattccagatgaaaggctctatcgaatgtttgtcaataaaaaagtaacaatgttgagaccaaaggaagatgagaactcttggtttaacttatttgtgattcatcagaacaggagtaaacatggaagtactaacttcattccagaacaatttttggatgacttcattgatcttgttatctggggccatgaacatgagtgtaaaatagctccaaccaaaaatgaacaacagctgttttatatctcacaacctggaagctcagtggttacttctctttccccaggagaagctgtaaagaaacatgttggtttgctgcgtattaaagggaggaagatgaatatgcataaaattcctcttcacacagtgcggcagtttttcatggaggatattgttctagctaatcatccagacatttttaacccagataatcctaaagtaacccaagccatacaaagcttctgtttggagaagattgaagaaatgcttgaaaatgctgaacgggaacgtctgggtaattctcaccagccagagaagcctcttgtacgactgcgagtggactatagtggaggttttgaacctttcagtgttcttcgctttagccagaaatttgtggatcgggtagctaatccaaaagacattatccattttttcaggcatagagaacaaaaggaaaaaacaggagaagagatcaactttgggaaacttatcacaaagccttcagaaggaacaactttaagggtagaagatcttgtaaaacagtactttcaaaccgcagagaagaatgtgcagctctcactgctaacagaaagagggatgggtgaagcagtacaagaatttgtggacaaggaggagaaagatgccattgaggaattagtgaaataccagttggaaaaaacacagcgatttcttaaagaacgtcatattgatgccctcgaagacaaaatcgatgaggaggtacgtcgtttcagagaaaccagacaaaaaaatactaatgaagaagatgatgaagtccgtgaggctatgaccagggccagagcactcagatctcagtcagaggagtctgcttctgcctttagtgctgatgaccttatgagtatagatttagcagaacagatggctaatgactctgatgatagcatctcagcagcaaccaacaaaggaagaggccgaggaagaggtcgaagaggtggaagagggcagaattcagcatcgagaggagggtctcaaagaggaagagcctttaaatctacaagacagcagccttcccgaaatgtcactactaagaattattcagaggtgattgaggtagatgaatcagatgtggaagaagacatttttcctaccacttcaaagacagatcaaaggtggtccagcacatcatccagcaaaatcatgtcccagagtcaagtatcgaaaggggttgattttgaatcaagtgaggatgatgatgatgatccttttatgaacactagttctttaagaagaaatagaagataatatatttaatggcactgagaaacatgcaagatacaggaaaaatgaaaatgttacaagctaagagtttacagtttaagattttaagtattgtttcctgagcataactccataagtaagaaatttctagttcacagacatacaatagcattgattcaccttgtttttttaacctggttgttgtagtaagagctttgtttcaatatcactcttgagtaaagattaaaataaagctaccattttacatttctatttcataatgaaaaactatgtcagtattttaatatggttacatttagccaaagttgagggaaagagcttataaaatttaacttcttcataattttagtaatttcctagaggttctgggttttctgaaagtaaaacaatttatgcgaacctatgtctaaattcactgtttgttactatgtatgtttttttccaatgcttcttataagactaaatgattagaagtacctaatagtttgaacagatatgtttttatttaaaagagtagaataacctttcagaattactgagttttttattccagttgtagcaaagatttcaaaagattgtgttcccattaagtggtagtaatttcctttattattctgtatccttaatggtgttctctctctctctctctctctctctctctccctctcccccccgttccccactcttcctttctcctttgctttttcttctctttcatacatatatgcgtgcctagttctaggaggaaacgggttaaaaattgttttaaactacatcttgaaaatattgaagaatttgttttaggtagagtggtcagttgaaccttacagtaaagtatagaaatatatttaatgtggaatgtcaatgccaggatttctcattaacaatattttatctcaactttggttcctgtgatacatttctgaatgggcaattccagaaatcttagtagcccatgttaagcttctattttttacttgttttcggggagaaataagaattagacatcttcagatttaagttaaataatcccattctttataatcctctgtaaaaagatccctgagattattccttcttctagttttatgcgacagctttactttaaaattcaagttatacatcttgggagtacaatggcccgacatttcttcataggtagaaacaaatacttgactcagtgatactcatgaccattagaatagtcatacctggaatgtgtcaaattataagagacagacacttggttagtggctgcctcatatagcacttttgaagaggcctaagtcaaaacttgcaatataacattctattgactttcttaaaaatattttttctgtacctaacttgagcataagggttatttgagcaagtaacattaactcagtggaaggcattgtcctgtgaaatattcttaggcagatctgcccacatctttattgaacttgaaatctaatatttctagtatttgaacaaagcagaaggttaagtcagggaagagcagtgctgtccatgatgtaatggaagctaccaggggaggcagtgtctggatgatgctgtgctacctacccctgcacaagccatgctggctcagtctgagctgtgggccacatcagctagtggctcttctcatgcatcagttaggtgggtctgggtgagagttatagtgagggaatggtcactaaagtatcctgacaagttcctaggaaaaaaggaataaagtttttttccttaaaaaaaaaaaaattgctcttggctgtgaaaagaggtactaaatgcgattcagttcaccgctaaggaaagtgatgacatagcagttacagagggtgataaatctctccagctaattcaggtcattttgtgaatactatgtatcaagccctgaaaatatggtaaataaaacgtgacagggaaacctttttttgattgaatattgttacatagttaaatgtgctatatatccttaatattttatattgatcctgcaaaatctgttggttttaggggagttttgttttttgtttctaacaattttcagacctgttggtataggaatgtagaagtctttcagatgatttgaaagcagctgcatttgctcttggaggctttgggagagcaggaatgaaaacattcagaggaagacatctgtagggaattcttctgttacttaccaaagaataagtgtctttctggtgttttatttcctatcataaaaatacaacagtgcatttacaaggttaaagattcctcgaagttctaggaaattcttgaaaatataagtggtgcttagaaaattcaagcatttaggaatgtgacctttaattcaggtatgtaaaagacttttttcccaaacttttaaaagtaggaaatacaataaatacagaaaagtcatatggttgaataaataattataaattgagcactgatggaatccctctacaggtcaagaaatagcgcagtgtcctggatgcccattatattgttttctcctttctgggtaacaagccctaacttctgtaatttaaaagctcctacttttgccacaaggtggtgcttctgccattagacgcagttaggaggatgcaactgcaaatctaaaattacgaagttagtgtagttgcaataaacttagaacatatgcattaatactaaacctatgcagtaataccataattagccttctaatcatgtaatttgctttacttaggtatttcatttggttcagcctgttatggaatttaccagcttgataaatttgcctataaagttttataaagaaaaggaatattttgttttcataaagaggaaaatccattcttagaaaaaaa

SEQ ID NO: 13 - DNA, 5141 bp, Homo sapiens
acgttatccatgaagtgtcgcgagagaaacggacgccgttctctcccgcggaattcaggtttacggccctgcgggttctcagagaatttctagaatttggaatcgagtgcattttctgacatttgagtacagtacccaggggttcttggagaagaacctggtcccagaggagcttgactgaccataaaaatgagtactgcagatgcacttgatgatgaaaacacatttaaaatattagttgcaacagatattcatcttggatttatggagaaagatgcagtcagaggaaatgatacgtttgtaacactcgatgaaattttaagacttgcccaggaaaatgaagtggattttattttgttaggtggtgatctttttcatgaaaataagccctcaaggaaaacattacatacctgcctcgagttattaagaaaatattgtatgggtgatcggcctgtccagtttgaaattctcagtgatcagtcagtcaactttggttttagtaagtttccatgggtgaactatcaagatggcaacctcaacatttcaattccagtgtttagtattcatggcaatcatgacgatcccacaggggcagatgcactttgtgccttggacattttaagttgtgctggatttgtaaatcactttggacgttcaatgtctgtggagaagatagacattagtccggttttgcttcaaaaaggaagcacaaagattgcgctatatggtttaggatccattccagatgaaaggctctatcgaatgtttgtcaataaaaaagtaacaatgttgagaccaaaggaagatgagaactcttggtttaacttatttgtgattcatcagaacaggagtaaacatggaagtactaacttcattccagaacaatttttggatgacttcattgatcttgttatctggggccatgaacatgagtgtaaaatagctccaaccaaaaatgaacaacagctgttttatatctcacaacctggaagctcagtggttacttctctttccccaggagaagctgtaaagaaacatgttggtttgctgcgtattaaagggaggaagatgaatatgcataaaattcctcttcacacagtgcggcagtttttcatggaggatattgttctagctaatcatccagacatttttaacccagataatcctaaagtaacccaagccatacaaagcttctgtttggagaagattgaagaaatgcttgaaaatgctgaacgggaacgtctgggtaattctcaccagccagagaagcctcttgtacgactgcgagtggactatagtggaggttttgaacctttcagtgttcttcgctttagccagaaatttgtggatcgggtagctaatccaaaagacattatccattttttcaggcatagagaacaaaaggaaaaaacaggagaagagatcaactttgggaaacttatcacaaagccttcagaaggaacaactttaagggtagaagatcttgtaaaacagtactttcaaaccgcagagaagaatgtgcagctctcactgctaacagaaagagggatgggtgaagcagtacaagaatttgtggacaaggaggagaaagatgccattgaggaattagtgaaataccagttggaaaaaacacagcgatttcttaaagaacgtcatattgatgccctcgaagacaaaatcgatgaggaggtacgtcgtttcagagaaaccagacaaaaaaatactaatgaagaagatgatgaagtccgtgaggctatgaccagggccagagcactcagatctcagtcagaggagtctgcttctgcctttagtgctgatgaccttatgagtatagatttagcagaacagatggctaatgactctgatgatagcatctcagcagcaaccaacaaaggaagaggccgaggaagaggtcgaagaggtggaagagggcagaattcagcatcgagaggagggtctcaaagaggaagagcagacactggtctggagacttctacccgtagcaggaactcaaagactgctgtgtcagcatctagaaatatgtctattatagatgcctttaaatctacaagacagcagccttcccgaaatgtcactactaagaattattcagaggtgattgaggtagatgaatcagatgtggaagaagacatttttcctaccacttcaaagacagatcaaaggtggtccagcacatcatccagcaaaatcatgtcccagagtcaagtatcgaaaggggttgattttgaatcaagtgaggatgatgatgatgatccttttatgaacactagttctttaagaagaaatagaagataatatatttaatggcactgagaaacatgcaagatacaggaaaaatgaaaatgttacaagctaagagtttacagtttaagattttaagtattgtttcctgagcataactccataagtaagaaatttctagttcacagacatacaatagcattgattcaccttgtttttttaacctggttgttgtagtaagagctttgtttcaatatcactcttgagtaaagattaaaataaagctaccattttacatttctatttcataatgaaaaactatgtcagtattttaatatggttacatttagccaaagttgagggaaagagcttataaaatttaacttcttcataattttagtaatttcctagaggttctgggttttctgaaagtaaaacaatttatgcgaacctatgtctaaattcactgtttgttactatgtatgtttttttccaatgcttcttataagactaaatgattagaagtacctaatagtttgaacagatatgtttttatttaaaagagtagaataacctttcagaattactgagttttttattccagttgtagcaaagatttcaaaagattgtgttcccattaagtggtagtaatttcctttattattctgtatccttaatggtgttctctctctctctctctctctctctctctccctctcccccccgttccccactcttcctttctcctttgctttttcttctctttcatacatatatgcgtgcctagttctaggaggaaacgggttaaaaattgttttaaactacatcttgaaaatattgaagaatttgttttaggtagagtggtcagttgaaccttacagtaaagtatagaaatatatttaatgtggaatgtcaatgccaggatttctcattaacaatattttatctcaactttggttcctgtgatacatttctgaatgggcaattccagaaatcttagtagcccatgttaagcttctattttttacttgttttcggggagaaataagaattagacatcttcagatttaagttaaataatcccattctttataatcctctgtaaaaagatccctgagattattccttcttctagttttatgcgacagctttactttaaaattcaagttatacatcttgggagtacaatggcccgacatttcttcataggtagaaacaaatacttgactcagtgatactcatgaccattagaatagtcatacctggaatgtgtcaaattataagagacagacacttggttagtggctgcctcatatagcacttttgaagaggcctaagtcaaaacttgcaatataacattctattgactttcttaaaaatattttttctgtacctaacttgagcataagggttatttgagcaagtaacattaactcagtggaaggcattgtcctgtgaaatattcttaggcagatctgcccacatctttattgaacttgaaatctaatatttctagtatttgaacaaagcagaaggttaagtcagggaagagcagtgctgtccatgatgtaatggaagctaccaggggaggcagtgtctggatgatgctgtgctacctacccctgcacaagccatgctggctcagtctgagctgtgggccacatcagctagtggctcttctcatgcatcagttaggtgggtctgggtgagagttatagtgagggaatggtcactaaagtatcctgacaagttcctaggaaaaaaggaataaagtttttttccttaaaaaaaaaaaaattgctcttggctgtgaaaagaggtactaaatgcgattcagttcaccgctaaggaaagtgatgacatagcagttacagagggtgataaatctctccagctaattcaggtcattttgtgaatactatgtatcaagccctgaaaatatggtaaataaaacgtgacagggaaacctttttttgattgaatattgttacatagttaaatgtgctatatatccttaatattttatattgatcctgcaaaatctgttggttttaggggagttttgttttttgtttctaacaattttcagacctgttggtataggaatgtagaagtctttcagatgatttgaaagcagctgcatttgctcttggaggctttgggagagcaggaatgaaaacattcagaggaagacatctgtagggaattcttctgttacttaccaaagaataagtgtctttctggtgttttatttcctatcataaaaatacaacagtgcatttacaaggttaaagattcctcgaagttctaggaaattcttgaaaatataagtggtgcttagaaaattcaagcatttaggaatgtgacctttaattcaggtatgtaaaagacttttttcccaaacttttaaaagtaggaaatacaataaatacagaaaagtcatatggttgaataaataattataaattgagcactgatggaatccctctacaggtcaagaaatagcgcagtgtcctggatgcccattatattgttttctcctttctgggtaacaagccctaacttctgtaatttaaaagctcctacttttgccacaaggtggtgcttctgccattagacgcagttaggaggatgcaactgcaaatctaaaattacgaagttagtgtagttgcaataaacttagaacatatgcattaatactaaacctatgcagtaataccataattagccttctaatcatgtaatttgctttacttaggtatttcatttggttcagcctgttatggaatttaccagcttgataaatttgcctataaagttttataaagaaaaggaatattttgttttcataaagaggaaaatccattcttagaaaaaaa

SEQ ID NO: 14 - DNA, 1651 bp, Homo sapiens
acagcagttacactgcggcgggcgtctgttctagtgtttgagccgtcgtgcttcaccggtctacctcgctagcatgtcgggccgcggcaagactggcggcaaggcccgcgccaaggccaagtcgcgctcgtcgcgcgccggcctccagttcccagtgggccgtgtacaccggctgctgcggaagggccactacgccgagcgcgttggcgccggcgcgccagtgtacctggcggcagtgctggagtacctcaccgctgagatcctggagctggcgggcaatgcggcccgcgacaacaagaagacgcgaatcatcccccgccacctgcagctggccatccgcaacgacgaggagctcaacaagctgctgggcggcgtgacgatcgcccagggaggcgtcctgcccaacatccaggccgtgctgctgcccaagaagaccagcgccaccgtggggccgaaggcgccctcgggcggcaagaaggccacccaggcctcccaggagtactaagagggcccgcgccgcggccggccgccaggcctccccatgccaccacaaaggcccttttaagggccaccaccgccctcatggaaagagctgagccgcttcagactgcggggcaagcgggccgcggctcccttcccctcccctcccctcgcccgccttcgccgcccggcctcgagtccccgcccgcccccgctcccgtcccgcaccgcctgccgcgtcggcctcgggccctgccctgtccgccgtccgccctccggtagggttcgggccttccggatgcggcttgggcgctcttcggggacctccgtggcgcggaagacccgagcctgccggggggaggccggcggcgccgcacctgcccgcctcggcgttcgtgactcagccgccccatcccgagtcgctaaggggctgcggggaggccgcagcaccttctggaagacttggccttccgctctgacgcagggccgaggtgggcagtccaggccgagaggccggcggccctgaaggtgagtgaggccctcggcagctgcagccggggtgtctggtacccccccggcgtggtgcttagcccaggactttcagacgcggccgctggccgggaggctttggtgggagagacgcgatcgccgatttcggtctggcgccccttctgcggccgggacccaggcctttcacatcagctctccctccatcttcattcataggtctgcgctggggccgggacgaagcacttggtaacaggcacatcttcctcccgagtgactgcctcctaggaggacatttaggggagggcagaggcctgcagtttggcttcacggctggctatgtggacagcaagagtcgttttcgcggaagccgactggcagccaggcctgtcgggccccccgacgccgccccatttcccttccagcaaactcaactcggcaatccaagcacctagataccagcacaagtcggttaatccctgtctggactgagcctccgttggcttctgaactggaattctgcagctaacccttccacgactagaaccttaggcattggggagttttagatggactaattttattaaaggattgttttttttttaaaaaaaaaaaaaaaaaaaaaaaaaaaaaaaaaaaaaaaaaaaaaaaaaaaaaaaaaaaa

SEQ ID NO: 15 - DNA, 3251 bp, Homo sapiens
gatttggctccgaggaggcggaagtgcagcacagaaagggggtccgtgggggacggtagaagcctggaggaggagcttgagtccagccactgtctgggtactgccagccatcgggcccaggtctctggggttgtcttaccgcagtgagtaccacgcggtactacagagaccggctgcccgtgtgcccggcaggtggagccgcccgcatcagcggcctcggggaatggaagcggagaacgcgggcagctattcccttcagcaagctcaagctttttatacgtttccatttcaacaactgatggctgaagctcctaatatggcagttgtgaatgaacagcaaatgccagaagaagttccagccccagctcctgctcaggaaccagtgcaagaggctccaaaaggaagaaaaagaaaacccagaacaacagaaccaaaacaaccagtggaacccaaaaaacctgttgagtcaaaaaaatctggcaagtctgcaaaatcaaaagaaaaacaagaaaaaattacagacacatttaaagtaaaaagaaaagtagaccgttttaatggtgtttcagaagctgaacttctgaccaagactctccccgatattttgaccttcaatctggacattgtcattattggcataaacccgggactaatggctgcttacaaagggcatcattaccctggacctggaaaccatttttggaagtgtttgtttatgtcagggctcagtgaggtccagctgaaccatatggatgatcacactctaccagggaagtatggtattggatttaccaacatggtggaaaggaccacgcccggcagcaaagatctctccagtaaagaatttcgtgaaggaggacgtattctagtacagaaattacagaaatatcagccacgaatagcagtgtttaatggaaaatgtatttatgaaatttttagtaaagaagtttttggagtaaaggttaagaacttggaatttgggcttcagccccataagattccagacacagaaactctctgctatgttatgccatcatccagtgcaagatgtgctcagtttcctcgagcccaagacaaagttcattactacataaaactgaaggacttaagagatcagttgaaaggcattgaacgaaatatggacgttcaagaggtgcaatatacatttgacctacagcttgcccaagaggatgcaaagaagatggctgttaaggaagaaaaatatgatccaggttatgaggcagcatatggtggtgcttacggagaaaatccatgcagcagtgaaccttgtggcttctcttcaaatgggctaattgagagcgtggagttaagaggagaatcagctttcagtggcattcctaatgggcagtggatgacccagtcatttacagaccaaattccttcctttagtaatcactgtggaacacaagaacaggaagaagaaagccatgcttaagaatggtgcttctcagctctgcttaaatgctgcagttttaatgcagttgtcaacaagtagaacctcagtttgctaactgaagtgttttattagtattttactctagtggtgtaattgtaatgtagaacagttgtgtggtagtgtgaaccgtatgaacctaagtagtttggaagaaaaagtagggtttttgtatactagcttttgtatttgaattaattatcattccagctttttatatactatatttcatttatgaagaaattgattttcttttgggagtcacttttaatctgtaattttaaaatacaagtctgaatatttatagttgattcttaactgcataaacctagatataccattatcccttttatacctaagaagggcatgctaataattaccactgtcaaagaggcaaaggtgttgatttttgtatatgaagttaagcctcagtggagtctcatttgttagtttttagtggtaactaagggtaaactcagggttccctgagctatatgcacactcagacctctttgctttaccagtggtgtttgtgagttgctcagtagtaaaaactggcccttacctgacagagccctggctttgacctgctcagccctgtgtgttaatcctctagtagccaattaactactctggggtggcaggttccagagaatgcagtagaccttttgccactcatctgtgttttacttgagacatgtaaatatgatagggaaggaactgaatttctccattcatatttataaccattctagttttatcttccttggctttaagagtgtgccatggaaagtgataagaaatgaacttctaggctaagcaaaaagatgctggagatatttgatactctcatttaaactggtgctttatgtacatgagatgtactaaaataagtaatatagaatttttcttgctaggtaaatccagtaagccaataattttaaagattctttatctgcatcattgctgtttgttactataaattaaatgaacctcatggaaaggttgaggtgtatacctttgtgattttctaatgagttttccatggtgctacaaataatccagactaccaggtctggtagatattaaagctgggtactaagaaatgttatttgcatcctctcagttactcctgaatattctgatttcatacgtacccagggagcatgctgttttgtcaatcaatataaaatatttatgaggtctcccccacccccaggaggttatatgattgctcttctctttataataagagaaacaaattcttattgtgaatcttaacatgctttttagctgtggctatgatggattttattttttcctaggtcaagctgtgtaaaagtcatttatgttatttaaatgatgtactgtactgctgtttacatggacgttttgtgcgggtgctttgaagtgccttgcatcagggattaggagcaattaaattattttttcacgggactgtgtaaagcatgtaactaggtattgctttggtatataactattgtagctttacaagagattgttttatttgaatggggaaaataccctttaaattatgacggacatccactagagatgggtttgaggattttccaagcgtgtaataatgatgtttttcctaacatgacagatgagtagtaaatgttgatatatcctatacatgacagtgtgagactttttcattaaataatattgaaagattttaaaattcatttgaaagtctgatggcttttacaataaaagatattaagaattgttatccttaacttaaaaaaaa

SEQ ID NO: 16 - DNA, 3448 bp, Homo sapiens
acggtttccccgcccctttcaggcctagcaggaaacgaagcggctctttccgctatctgccgcttgtccaccggaagcgagttgcgacacggcaggttcccgcccggaagaagcgaccaaagcgcctgaggaccggcaacatggtgcggtcggggaataaggcagctgttgtgctgtgtatggacgtgggctttaccatgagtaactccattcctggtatagaatccccatttgaacaagcaaagaaggtgataaccatgtttgtacagcgacaggtgtttgctgagaacaaggatgagattgctttagtcctgtttggtacagatggcactgacaatcccctttctggtggggatcagtatcagaacatcacagtgcacagacatctgatgctaccagattttgatttgctggaggacattgaaagcaaaatccaaccaggttctcaacaggctgacttcctggatgcactaatcgtgagcatggatgtgattcaacatgaaacaataggaaagaagtttgagaagaggcatattgaaatattcactgacctcagcagccgattcagcaaaagtcagctggatattataattcatagcttgaagaaatgtgacatctccctgcaattcttcttgcctttctcacttggcaaggaagatggaagtggggacagaggagatggcccctttcgcttaggtggccatgggccttcctttccactaaaaggaattaccgaacagcaaaaagaaggtcttgagatagtgaaaatggtgatgatatctttagaaggtgaagatgggttggatgaaatttattcattcagtgagagtctgagaaaactgtgcgtcttcaagaaaattgagaggcattccattcactggccctgccgactgaccattggctccaatttgtctataaggattgcagcctataaatcgattctacaggagagagttaaaaagacttggacagttgtggatgcaaaaaccctaaaaaaagaagatatacaaaaagaaacagtttattgcttaaatgatgatgatgaaactgaagttttaaaagaggatattattcaagggttccgctatggaagtgatatagttcctttctctaaagtggatgaggaacaaatgaaatataaatcggaggggaagtgcttctctgttttgggattttgtaaatcttctcaggttcagagaagattcttcatgggaaatcaagttctaaaggtctttgcagcaagagatgatgaggcagctgcagttgcactttcctccctgattcatgctttggatgacttagacatggtggccatagttcgatatgcttatgacaaaagagctaatcctcaagtcggcgtggcttttcctcatatcaagcataactatgagtgtttagtgtatgtgcagctgcctttcatggaagacttgcggcaatacatgttttcatccttgaaaaacagtaagaaatatgctcccaccgaggcacagttgaatgctgttgatgctttgattgactccatgagcttggcaaagaaagatgagaagacagacacccttgaagacttgtttccaaccaccaaaatcccaaatcctcgatttcagagattatttcagtgtctgctgcacagagctttacatccccgggagcctctacccccaattcagcagcatatttggaatatgctgaatcctcccgctgaggtgacaacaaaaagtcagattcctctctctaaaataaagaccctttttcctctgattgaagccaagaaaaaggatcaagtgactgctcaggaaattttccaagacaaccatgaagatggacctacagctaaaaaattaaagactgagcaagggggagcccacttcagcgtctccagtctggctgaaggcagtgtcacctctgttggaagtgtgaatcctgctgaaaacttccgtgttctagtgaaacagaagaaggccagctttgaggaagcgagtaaccagctcataaatcacatcgaacagtttttggatactaatgaaacaccgtattttatgaagagcatagactgcatccgagccttccgggaagaagccattaagttttcagaagagcagcgctttaacaacttcctgaaagcccttcaagagaaagtggaaattaaacaattaaatcatttctgggaaattgttgtccaggatggaattactctgatcaccaaagaggaagcctctggaagttctgtcacagctgaggaagccaaaaagtttctggcccccaaagacaaaccaagtggagacacagcagctgtatttgaagaaggtggtgatgtggacgatttattggacatgatataggtcgtggatgtatggggaatctaagagagctgccatcgctgtgatgctgggagttctaacaaaacaagttggatgcggccattcaaggggagccaaaatctcaagaaattcccagcaggttacctggaggcggatcatctaattctctgtggaatgaatacacacatatatattacaagggataatttagaccccatacaagtttataaagagtcattgttattttctggttggtgtattattttttctgtggtcttactgatctttgtatattacatacatgctttgaagtttctggaaagtagatcttttcttgacctagtatatcagtgacagttgcagcccttgtgatgtgattagtgtctcatgtggaaccatggcatggttattgatgagtttcttaaccctttccagagtcctcctttgcctgatcctccaacagctgtcacaacttgtgttgagcaagcagtagcatttgcttcctcccaacaagcagctgggttaggaaaaccatgggtaaggacggactcacttctctttttagttgaggccttctagttaccacattactctgcctctgtatataggtggttttctttaagtggggtgggaaggggagcacaatttcccttcatactccttttaagcagtgagttatggtggtggtctcatgaagaaaagaccttttggcccaatctctgccatatcagtgaacctttagaaactcaaaaactgagaaatttactacagtagttagaattatatcacttcactgttctctacttgcaagcctcaaagagagaaagtttcgttatattaaaacacttaggtaacttttcggtctttcccatttctacctaagtcagctttcatctttgtggatggtgtctcctttactaaataagaaaataacaaagcccttattctctttttttcttgtcctcattcttgccttgagttccagttcctctttggtgtacagacttcttggtacccagtcacctctgtcttcagcaccctcataagtcgtcactaatacacagttttgtacatgtaacattaaaggcataaatgactcatctctctgtgaaaaaaaaaaaaaaaaaaaa

SEQ ID NO: 17 - DNA, 4998 bp, Homo sapiens
cgaggaagtgcggcgtgaagttgtggagctgagattgcccgccgctggggacccggagcccaggagcgccccttcccaggcggccccttccggcgccgcgcctgtgcctgccctcgccgcgccccgcgcccgcagcctggtccagcctgagccatggggccggagccgcagtgatcatcatggagctggcggcctggtgccgttgggggttcctcctcgccctcctgtcccccggagccgcgggtacccaagtgtgtaccggtaccgacatgaagttgcgactccctgccagtcctgagacccacctggacatgcttcgccacctctaccagggctgtcaggtggtgcagggcaatttggagcttacctacctgcccgccaatgccagcctctcattcctgcaggacatccaggaagtccagggatacatgctcatcgctcacaaccgagtgaaacacgtcccactgcagaggttgcgcatcgtgagagggactcagctctttgaggacaagtatgccctggctgtgctagacaaccgagaccctttggacaacgtcaccaccgccgccccaggcagaaccccagaagggctgcgggagctgcagcttcgaagtctcacagagatcttgaagggaggagttttgatccgtgggaaccctcagctctgctaccaggacatggttttgtggaaggatgtcctccgtaagaataaccagctggctcctgtcgacatggacaccaatcgttcccgggcctgtccaccttgtgccccaacctgcaaagacaatcactgttggggtgagagtcctgaagactgtcagatcttgactggcaccatctgtactagtggctgtgcccggtgcaagggccggctgcccactgactgttgccatgagcagtgtgctgcaggctgcacgggtcccaagcattctgactgcctggcctgcctccacttcaatcatagtggtatctgtgagctgcactgcccggccctcatcacctacaacacagacaccttcgagtccatgctcaaccctgagggtcgctacacctttggtgccagctgtgtgaccacctgcccctacaactacctctccacggaagtgggatcctgcactctggtctgtcccccgaacaaccaagaggtcacagctgaggacggaacacagcggtgtgagaaatgcagcaagccctgtgctggagtatgctatggtctgggcatggagcacctccgaggggcgagggccatcaccagtgacaatatccaggagtttgctggctgcaagaagatctttgggagcctggcatttttgccggagagctttgatgggaacccctcctccggcgttgccccactgaagccagagcatctccaagtgttcgaaaccctggaggagatcacaggttacctatacatttcagcatggccagagagcttccaagacctcagtgtcttccagaaccttcgggtcattcggggacggattctccatgatggtgcttactcattgacgttgcaaggcctggggattcactcactggggctacgctcactgcgggagctgggcagtggattggctctcattcaccgcaacacccatctctgctttgtaaacactgtaccttgggaccagctcttccggaacccgcaccaggccctactccacagtgggaaccggccagaagaggcatgtggtcttgagggcttggtctgtaactcactgtgtgcccgtgggcactgctgggggccagggcccacccagtgtgtcaactgcagtcagttcctccggggccaggagtgtgtggaggagtgccgagtatggaaggggctccccagggagtatgtgaggggcaagcactgtctgccatgccaccccgagtgtcagcctcaaaacagctcggagacctgctatggatcggaggctgaccagtgtgaggcttgtgcccactacaaggactcatcttcctgtgtggctcgctgccccagtggtgtgaagccagacctctcctacatgcctatctggaagtacccggatgaggagggcatatgtcagccatgccccatcaactgcacccactcatgtgtggacctggacgaacgaggctgcccagcagagcagagagccagcccagtgacattcatcattgcaactgtggtgggcgtcctgttgttcctgatcatagtggtggtcattggaatcctaatcaaacgaaggcgacagaagatccggaagtataccatgcgtaggctgctgcaggagaccgagctggtggagccgctgacgcccagtggagctgtgcccaaccaggctcagatgcggatcctaaaggagacagagctaaggaagctgaaggtgcttgggtcaggagccttcggcactgtctacaagggcatctggatcccagatggggagaacgtgaaaatccccgtggccatcaaggtgttgagggaaaacacatctcctaaagctaacaaagaaatcctagatgaagcgtacgtcatggctggtgtgggttctccatatgtgtcccgcctcctgggcatctgcctgacatccacagtgcagctggtgacacagcttatgccctatggctgccttctggaccatgtccgagaacaccgaggtcgcttaggctcccaggacctgctcaactggtgtgttcagattgccaaggggatgagctacctggaggaagttcggcttgttcacagggacctagctgcccgaaacgtgctagtcaagagtcccaaccacgtcaagattaccgacttcgggctggcacggctgctggacattgatgagactgaataccatgcagatgggggcaaggtgcccatcaagtggatggcattggaatctattctcagacgccggttcacccatcagagtgatgtgtggagctatggtgtgactgtgtgggagctgatgacctttggggccaaaccttacgatgggatcccagctcgggagatccctgatttgctggagaagggagaacgcctacctcagcctccaatctgcaccatcgacgtctacatgatcatggtcaaatgttggatgattgactccgaatgtcgcccgagattccgggagttggtatcagaattctcccgtatggcaagggacccccagcgctttgtggtcatccagaacgaggacttaggcccctccagccccatggacagcaccttctaccgttcactgctggaggatgatgacatgggggagctggtcgatgctgaagagtacctggtaccccagcagggattcttctccccagaccctgccctaggtactgggagcacagcccaccgcagacaccgcagctcgtcggccaggagtggcggtggtgagctgacactgggcctggagccctcggaagaagagccccccagatctccactggctccctccgaaggggctggctccgatgtgtttgatggtgacctggcagtgggggtaaccaaaggactgcagagcctctctccacatgacctcagccctctacagcggtacagtgaggatcccacattacctctgccccccgagactgatggctacgttgctcccctggcctgcagcccccagcccgagtatgtgaaccagccagaggttcggcctcagtctcccttgaccccagagggtcctccgcctcccatccgacctgctggtgctactctagaaagacccaagactctctctcctgggaaaaatggggttgtcaaagacgtttttgcctttgggggtgctgtggagaaccctgaatacttagcacccagagcaggcactgcctctcagccccacccttctcctgccttcagcccagcctttgacaacctctattactgggaccagaactcatcggagcagggtcctccaccaagtacctttgaagggacccccactgcagagaaccctgagtacctaggcctggatgtgccagtatgaggtcacatgtgcagacatcctctgtcttcagagtggggaaggaaggcctaacttgtggtctccatcgcccgccacaaagcagggagaaggtcctctggccacatgacatccagggcagccggctatgccaggaacgtgccctgaggaacctcgctcgatgcttcaatcctgagtggttaagagggccccgcctggccggaagagacagcacactgttcagccccagaggattacagaccctgactgccctgacagactgtagggtccagtgggtattccttacctggcctggctctcttggttctgaagactgagggaagctcagcctgcaagggaggaggccccaggtgaatatcctgggagcaggacaccccactaggactgaggcacgtgcatcccaagagggggacagcacttgcacccagactggtctttgtacagagtttattttgttctgtttttacttttgttttttgttttttttttaaagatgaaataaggatacagtgggagagtgggtgttatatgaaagtcggggggtgctgtcccctttctccatttgcaatgagatttgtaaaataactggaccccagcctatgtctgagagtggtcccgggccgggtcaaaccgtattgctcatctgacacacagctcctcctggagtgagtgtgtagagatcttccaaaagtttgagacaatttggctttgggcttgagggactggggagttaggattccttctgaaggccctttggcaacagggtcattctccgttggacacactcataccaaggctacccccagaatactccgttggacacactcattccaaggctacccccagaatgaagtcctgtcctcccagtgggagaggggagcttgtggagagcattgccatgtgacttgttttccttgccttagaaagaagtatccatccaggaaaaccccacccactaggtgttagtcccacccactaggtgttagcagggccagactgacctgtgtgccccccgcacaggctggacataaacacacgccagttgacacaa

SEQ ID NO: 18 - DNA, 4624 bp, Homo sapiens
ggaggaggtggaggaggagggctgcttgaggaagtataagaatgaagttgtgaagctgagattcccctccattgggaccggagaaaccaggggagccccccgggcagccgcgcgccccttcccacggggccctttactgcgccgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaacagcta

SEQ ID NO: 19 - protein, 1863 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 20 - protein, 1884 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKDSHIHGQRNNSMFSKRPREHISVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 21 - protein, 1816 aa, Homo sapiens
MLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 22 - protein, 700 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGEAASGCESETSVSEDCSGLSSQSDILTTQQRDTMQHNLIKLQQEMAELEAVLEQHGSQPSNSYPSIISDSSALEDLRNPEQSTSEKVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGV

SEQ ID NO: 23 - protein, 699 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGEAASGCESETSVSEDCSGLSSQSDILTTQQRDTMQHNLIKLQQEMAELEAVLEQHGSQPSNSYPSIISDSSALEDLRNPEQSTSEKVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTGCPPNCGCAARCLDRGQWLPCNWADV

SEQ ID NO: 24 - protein, 3418 aa, Homo sapiens
MPIGSKERPTFFEIFKTRCNKADLGPISLNWFEELSSEAPPYNSEPAEESEHKNNNYEPNLFKTPQRKPSYNQLASTPIIFKEQGLTLPLYQSPVKELDKFKLDLGRNVPNSRHKSLRTVKTKMDQADDVSCPLLNSCLSESPVVLQCTHVTPQRDKSVVCGSLFHTPKFVKGRQTPKHISESLGAEVDPDMSWSSSLATPPTLSSTVLIVRNEEASETVFPHDTTANVKSYFSNHDESLKKNDRFIASVTDSENTNQREAASHGFGKTSGNSFKVNSCKDHIGKSMPNVLEDEVYETVVDTSEEDSFSLCFSKCRTKNLQKVRTSKTRKKIFHEANADECEKSKNQVKEKYSFVSEVEPNDTDPLDSNVANQKPFESGSDKISKEVVPSLACEWSQLTLSGLNGAQMEKIPLLHISSCDQNISEKDLLDTENKRKKDFLTSENSLPRISSLPKSEKPLNEETVVNKRDEEQHLESHTDCILAVKQAISGTSPVASSFQGIKKSIFRIRESPKETFNASFSGHMTDPNFKKETEASESGLEIHTVCSQKEDSLCPNLIDNGSWPATTTQNSVALKNAGLISTLKKKTNKFIYAIHDETSYKGKKIPKDQKSELINCSAQFEANAFEAPLTFANADSGLLHSSVKRSCSQNDSEEPTLSLTSSFGTILRKCSRNETCSNNTVISQDLDYKEAKCNKEKLQLFITPEADSLSCLQEGQCENDPKSKKVSDIKEEVLAAACHPVQHSKVEYSDTDFQSQKSLLYDHENASTLILTPTSKDVLSNLVMISRGKESYKMSDKLKGNNYESDVELTKNIPMEKNQDVCALNENYKNVELLPPEKYMRVASPSRKVQFNQNTNLRVIQKNQEETTSISKITVNPDSEELFSDNENNFVFQVANERNNLALGNTKELHETDLTCVNEPIFKNSTMVLYGDTGDKQATQVSIKKDLVYVLAEENKNSVKQHIKMTLGQDLKSDISLNIDKIPEKNNDYMNKWAGLLGPISNHSFGGSFRTASNKEIKLSEHNIKKSKMFFKDIEEQYPTSLACVEIVNTLALDNQKKLSKPQSINTVSAHLQSSVVVSDCKNSHITPQMLFSKQDFNSNHNLTPSQKAEITELSTILEESGSQFEFTQFRKPSYILQKSTFEVPENQMTILKTTSEECRDADLHVIMNAPSIGQVDSSKQFEGTVEIKRKFAGLLKNDCNKSASGYLTDENEVGFRGFYSAHGTKLNVSTEALQKAVKLFSDIENISEETSAEVHPISLSSSKCHDSVVSMFKIENHNDKTVSEKNNKCQLILQNNIEMTTGTFVEEITENYKRNTENEDNKYTAASRNSHNLEFDGSDSSKNDTVCIHKDETDLLFTDQHNICLKLSGQFMKEGNTQIKEDLSDLTFLEVAKAQEACHGNTSNKEQLTATKTEQNIKDFETSDTFFQTASGKNISVAKESFNKIVNFFDQKPEELHNFSLNSELHSDIRKNKMDILSYEETDIVKHKILKESVPVGTGNQLVTFQGQPERDEKIKEPTLLGFHTASGKKVKIAKESLDKVKNLFDEKEQGTSEITSFSHQWAKTLKYREACKDLELACETIEITAAPKCKEMQNSLNNDKNLVSIETVVPPKLLSDNLCRQTENLKTSKSIFLKVKVHENVEKETAKSPATCYTNQSPYSVIENSALAFYTSCSRKTSVSQTSLLEAKKWLREGIFDGQPERINTADYVGNYLYENNSNSTIAENDKNHLSEKQDTYLSNSSMSNSYSYHSDEVYNDSGYLSKNKLDSGIEPVLKNVEDQKNTSFSKVISNVKDANAYPQTVNEDICVEELVTSSSPCKNKNAAIKLSISNSNNFEVGPPAFRIASGKIVCVSHETIKKVKDIFTDSFSKVIKENNENKSKICQTKIMAGCYEALDDSEDILHNSLDNDECSTHSHKVFADIQSEEILQHNQNMSGLEKVSKISPCDVSLETSDICKCSIGKLHKSVSSANTCGIFSTASGKSVQVSDASLQNARQVFSEIEDSTKQVFSKVLFKSNEHSDQLTREENTAIRTPEHLISQKGFSYNVVNSSAFSGFSTASGKQVSILESSLHKVKGVLEEFDLIRTEHSLHYSPTSRQNVSKILPRVDKRNPEHCVNSEMEKTCSKEFKLSNNLNVEGGSSENNHSIKVSPYLSQFQQDKQQLVLGTKVSLVENIHVLGKEQASPKNVKMEIGKTETFSDVPVKTNIEVCSTYSKDSENYFETEAVEIAKAFMEDDELTDSKLPSHATHSLFTCPENEEMVLSNSRIGKRRGEPLILVGEPSIKRNLLNEFDRIIENQEKSLKASKSTPDGTIKDRRLFMHHVSLEPITCVPFRTTKERQEIQNPNFTAPGQEFLSKSHLYEHLTLEKSSSNLAVSGHPFYQVSATRNEKMRHLITTGRPTKVFVPPFKTKSHFHRVEQCVRNINLEENRQKQNIDGHGSDDSKNKINDNEIHQFNKNNSNQAAAVTFTKCEEEPLDLITSLQNARDIQDMRIKKKQRQRVFPQPGSLYLAKTSTLPRISLKAAVGGQVPSACSHKQLYTYGVSKHCIKINSKNAESFQFHTEDYFGKESLWTGKGIQLADGGWLIPSNDGKAGKEEFYRALCDTPGVDPKLISRIWVYNHYRWIIWKLAAMECAFPKEFANRCLSPERVLLQLKYRYDTEIDRSRRSAIKKIMERDDTAAKTLVLCVSDIISLSANISETSSNKTSSADTQKVAIIELTDGWYAVKAQLDPPLLAVLKNGRLTVGQKIILHGAELVGSPDACTPLEAPESLMLKISANSTRPARWYTKLGFFPDPRPFPLPLSSLFSDGGNVGCVDVIIQRAYPIQWMEKTSSGLYIFRNEREEEKEAAKYVEAQQKRLEALFTKIQEEFEEHEENTTKPYLPSRALTRQQVRALQDGAELYEAVKNAADPAYLEGYFSEEQLRALNNHRQMLNDKKQAQIQLEIRKAMESAEQKEQGLSRDVTTVWKLRIVSYSKKEKDSVILSIWRPSSDLYSLLTEGKRYRIYHLATSKSKSKSERANIQLAATKKTQYQQLPVSDEILFQIYQPREPLHFSKFLDPDFQPSCSEVDLIGFVVSVVKKTGLAPFVYLSDECYNLLAIKFWIDLNEDIIKPHMLIAASNLQWRPESKSGLLTLFAGDFSVFSASPKEGHFQETFNKMKNTVENIDILCNEAENKLMHILHANDPKWSTPTKDCTSGPYTAQIIPGTGNKLLMSSPNCEIYYQSPLSLCMAKRKSVSTPVSAQMTSKSCKGEKEIDDQKNCKKRRALDFLSRLPLPPPVSPICTFVSPAAQKAFQPPRSCGTKYETPIKKKELNSPQMTPFKKFNEISLLESNSIADEELALINTQALLSGSTGEKQFISVSESTRTAPTSSEDYLRLKRRCTTSLIKEQESSQASTEECEKNKQDTITTKKYI

SEQ ID NO: 25 - protein, 476 aa, Homo sapiens
MAVPFVEDWDLVQTLGEGAYGEVQLAVNRVTEEAVAVKIVDMKRAVDCPENIKKEICINKMLNHENVVKFYGHRREGNIQYLFLEYCSGGELFDRIEPDIGMPEPDAQRFFHQLMAGVVYLHGIGITHRDIKPENLLLDERDNLKISDFGLATVFRYNNRERLLNKMCGTLPYVAPELLKRREFHAEPVDVWSCGIVLTAMLAGELPWDQPSDSCQEYSDWKEKKTYLNPWKKIDSAPLALLHKILVENPSARITIPDIKKDRWYNKPLKKGAKRPRVTSGGVSESPSGFSKHIQSNLDFSPVNSASSEENVKYSSSQPEPRTGLSLWDTSPSYIDKLVQGISFSQPTCPDHMLLNSQLLGTPGSSQNPWQRLVKRMTRFFTKLDADKSYQCLKETCEKLGYQWKKSCMNQVTISTTDRRNNKLIFKVNLLEMDDKILVDFRLSKGDGLEFKRHFLKIKGKLIDIVSSQKIWLPAT

SEQ ID NO: 26 - protein, 442 aa, Homo sapiens
MAVPFVEDWDLVQTLGEGAYGEVQLAVNRVTEEAVAVKIVDMKRAVDCPENIKKEICINKMLNHENVVKFYGHRREGNIQYLFLEYCSGGELFDRIEPDIGMPEPDAQRFFHQLMAGVVYLHGIGITHRDIKPENLLLDERDNLKISDFGLATVFRYNNRERLLNKMCGTLPYVAPELLKRREFHAEPVDVWSCGIVLTAMLAGELPWDQPSDSCQEYSDWKEKKTYLNPWKKIDSAPLALLHKILVENPSARITIPDIKKDRWYNKPLKKGAKRPRVTSGGVSESPSGFSKHIQSNLDFSPVNSASSEENVKYSSSQPEPRTGLSLWDTSPSYIDKLVQGISFSQPTCPDHMLLNSQLLGTPGSSQNPWQRLVKRMTRFFTKLDADKSYQCLKETCEKLGYQWKKSCMNQGDGLEFKRHFLKIKGKLIDIVSSQKIWLPAT

SEQ ID NO: 27 - protein, 586 aa, Homo sapiens
MSRESDVEAQQSHGSSACSQPHGSVTQSQGSSSQSQGISSSSTSTMPNSSQSSHSSSGTLSSLETVSTQELYSIPEDQEPEDQEPEEPTPAPWARLWALQDGFANLETESGHVTQSDLELLLSSDPPASASQSAGIRGVRHHPRPVCSLKCVNDNYWFGRDKSCEYCFDEPLLKRTDKYRTYSKKHFRIFREVGPKNSYIAYIEDHSGNGTFVNTELVGKGKRRPLNNNSEIALSLSRNKVFVFFDLTVDDQSVYPKALRDEYIMSKTLGSGACGEVKLAFERKTCKKVAIKIISKRKFAIGSAREADPALNVETEIEILKKLNHPCIIKIKNFFDAEDYYIVLELMEGGELFDKVVGNKRLKEATCKLYFYQMLLAVQYLHENGIIHRDLKPENVLLSSQEEDCLIKITDFGHSKILGETSLMRTLCGTPTYLAPEVLVSVGTAGYNRAVDCWSLGVILFICLSGYPPFSEHRTQVSLKDQITSGKYNFIPEVWAEVSEKALDLVKKLLVVDPKARFTTEEALRHPWLQDEDMKRKFQDLLSEENESTALPQVLAQPSTSRKRPREGEAEGAETTKRPAVCAAVL

SEQ ID NO: 28 - protein, 543 aa, Homo sapiens
MSRESDVEAQQSHGSSACSQPHGSVTQSQGSSSQSQGISSSSTSTMPNSSQSSHSSSGTLSSLETVSTQELYSIPEDQEPEDQEPEEPTPAPWARLWALQDGFANLECVNDNYWFGRDKSCEYCFDEPLLKRTDKYRTYSKKHFRIFREVGPKNSYIAYIEDHSGNGTFVNTELVGKGKRRPLNNNSEIALSLSRNKVFVFFDLTVDDQSVYPKALRDEYIMSKTLGSGACGEVKLAFERKTCKKVAIKIISKRKFAIGSAREADPALNVETEIEILKKLNHPCIIKIKNFFDAEDYYIVLELMEGGELFDKVVGNKRLKEATCKLYFYQMLLAVQYLHENGIIHRDLKPENVLLSSQEEDCLIKITDFGHSKILGETSLMRTLCGTPTYLAPEVLVSVGTAGYNRAVDCWSLGVILFICLSGYPPFSEHRTQVSLKDQITSGKYNFIPEVWAEVSEKALDLVKKLLVVDPKARFTTEEALRHPWLQDEDMKRKFQDLLSEENESTALPQVLAQPSTSRKRPREGEAEGAETTKRPAVCAAVL

SEQ ID NO: 29 - protein, 514 aa, Homo sapiens
MSRESDVEAQQSHGSSACSQPHGSVTQSQGSSSQSQGISSSSTSTMPNSSQSSHSSSGTLSSLETVSTQELYSIPEDQEPEDQEPEEPTPAPWARLWALQDGFANLECVNDNYWFGRDKSCEYCFDEPLLKRTDKYRTYSKKHFRIFREVGPKNSYIAYIEDHSGNGTFVNTELVGKGKRRPLNNNSEIALSLSRNKVFVFFDLTVDDQSVYPKALRDEYIMSKTLGSGACGEVKLAFERKTCKKVAIKIISKRKFAIGSAREADPALNVETEIEILKKLNHPCIIKIKNFFDAEDYYIVLELMEGGELFDKVVGNKRLKEATCKLYFYQMLLAVQITDFGHSKILGETSLMRTLCGTPTYLAPEVLVSVGTAGYNRAVDCWSLGVILFICLSGYPPFSEHRTQVSLKDQITSGKYNFIPEVWAEVSEKALDLVKKLLVVDPKARFTTEEALRHPWLQDEDMKRKFQDLLSEENESTALPQVLAQPSTSRKRPREGEAEGAETTKRPAVCAAVL

SEQ ID NO: 30 - protein, 680 aa, Homo sapiens
MSTADALDDENTFKILVATDIHLGFMEKDAVRGNDTFVTLDEILRLAQENEVDFILLGGDLFHENKPSRKTLHTCLELLRKYCMGDRPVQFEILSDQSVNFGFSKFPWVNYQDGNLNISIPVFSIHGNHDDPTGADALCALDILSCAGFVNHFGRSMSVEKIDISPVLLQKGSTKIALYGLGSIPDERLYRMFVNKKVTMLRPKEDENSWFNLFVIHQNRSKHGSTNFIPEQFLDDFIDLVIWGHEHECKIAPTKNEQQLFYISQPGSSVVTSLSPGEAVKKHVGLLRIKGRKMNMHKIPLHTVRQFFMEDIVLANHPDIFNPDNPKVTQAIQSFCLEKIEEMLENAERERLGNSHQPEKPLVRLRVDYSGGFEPFSVLRFSQKFVDRVANPKDIIHFFRHREQKEKTGEEINFGKLITKPSEGTTLRVEDLVKQYFQTAEKNVQLSLLTERGMGEAVQEFVDKEEKDAIEELVKYQLEKTQRFLKERHIDALEDKIDEEVRRFRETRQKNTNEEDDEVREAMTRARALRSQSEESASAFSADDLMSIDLAEQMANDSDDSISAATNKGRGRGRGRRGGRGQNSASRGGSQRGRAFKSTRQQPSRNVTTKNYSEVIEVDESDVEEDIFPTTSKTDQRWSSTSSSKIMSQSQVSKGVDFESSEDDDDDPFMNTSSLRRNRR

SEQ ID NO: 31 - protein, 708 aa, Homo sapiens
MSTADALDDENTFKILVATDIHLGFMEKDAVRGNDTFVTLDEILRLAQENEVDFILLGGDLFHENKPSRKTLHTCLELLRKYCMGDRPVQFEILSDQSVNFGFSKFPWVNYQDGNLNISIPVFSIHGNHDDPTGADALCALDILSCAGFVNHFGRSMSVEKIDISPVLLQKGSTKIALYGLGSIPDERLYRMFVNKKVTMLRPKEDENSWFNLFVIHQNRSKHGSTNFIPEQFLDDFIDLVIWGHEHECKIAPTKNEQQLFYISQPGSSVVTSLSPGEAVKKHVGLLRIKGRKMNMHKIPLHTVRQFFMEDIVLANHPDIFNPDNPKVTQAIQSFCLEKIEEMLENAERERLGNSHQPEKPLVRLRVDYSGGFEPFSVLRFSQKFVDRVANPKDIIHFFRHREQKEKTGEEINFGKLITKPSEGTTLRVEDLVKQYFQTAEKNVQLSLLTERGMGEAVQEFVDKEEKDAIEELVKYQLEKTQRFLKERHIDALEDKIDEEVRRFRETRQKNTNEEDDEVREAMTRARALRSQSEESASAFSADDLMSIDLAEQMANDSDDSISAATNKGRGRGRGRRGGRGQNSASRGGSQRGRADTGLETSTRSRNSKTAVSASRNMSIIDAFKSTRQQPSRNVTTKNYSEVIEVDESDVEEDIFPTTSKTDQRWSSTSSSKIMSQSQVSKGVDFESSEDDDDDPFMNTSSLRRNRR

SEQ ID NO: 32 - protein, 143 aa, Homo sapiens
MSGRGKTGGKARAKAKSRSSRAGLQFPVGRVHRLLRKGHYAERVGAGAPVYLAAVLEYLTAEILELAGNAARDNKKTRIIPRHLQLAIRNDEELNKLLGGVTIAQGGVLPNIQAVLLPKKTSATVGPKAPSGGKKATQASQEY

SEQ ID NO: 33 - protein, 410 aa, Homo sapiens
MEAENAGSYSLQQAQAFYTFPFQQLMAEAPNMAVVNEQQMPEEVPAPAPAQEPVQEAPKGRKRKPRTTEPKQPVEPKKPVESKKSGKSAKSKEKQEKITDTFKVKRKVDRFNGVSEAELLTKTLPDILTFNLDIVIIGINPGLMAAYKGHHYPGPGNHFWKCLFMSGLSEVQLNHMDDHTLPGKYGIGFTNMVERTTPGSKDLSSKEFREGGRILVQKLQKYQPRIAVFNGKCIYEIFSKEVFGVKVKNLEFGLQPHKIPDTETLCYVMPSSSARCAQFPRAQDKVHYYIKLKDLRDQLKGIERNMDVQEVQYTFDLQLAQEDAKKMAVKEEKYDPGYEAAYGGAYGENPCSSEPCGFSSNGLIESVELRGESAFSGIPNGQWMTQSFTDQIPSFSNHCGTQEQEEESHA

SEQ ID NO: 34 - protein, 732 aa, Homo sapiens
MVRSGNKAAVVLCMDVGFTMSNSIPGIESPFEQAKKVITMFVQRQVFAENKDEIALVLFGTDGTDNPLSGGDQYQNITVHRHLMLPDFDLLEDIESKIQPGSQQADFLDALIVSMDVIQHETIGKKFEKRHIEIFTDLSSRFSKSQLDIIIHSLKKCDISLQFFLPFSLGKEDGSGDRGDGPFRLGGHGPSFPLKGITEQQKEGLEIVKMVMISLEGEDGLDEIYSFSESLRKLCVFKKIERHSIHWPCRLTIGSNLSIRIAAYKSILQERVKKTWTVVDAKTLKKEDIQKETVYCLNDDDETEVLKEDIIQGFRYGSDIVPFSKVDEEQMKYKSEGKCFSVLGFCKSSQVQRRFFMGNQVLKVFAARDDEAAAVALSSLIHALDDLDMVAIVRYAYDKRANPQVGVAFPHIKHNYECLVYVQLPFMEDLRQYMFSSLKNSKKYAPTEAQLNAVDALIDSMSLAKKDEKTDTLEDLFPTTKIPNPRFQRLFQCLLHRALHPREPLPPIQQHIWNMLNPPAEVTTKSQIPLSKIKTLFPLIEAKKKDQVTAQEIFQDNHEDGPTAKKLKTEQGGAHFSVSSLAEGSVTSVGSVNPAENFRVLVKQKKASFEEASNQLINHIEQFLDTNETPYFMKSIDCIRAFREEAIKFSEEQRFNNFLKALQEKVEIKQLNHFWEIVVQDGITLITKEEASGSSVTAEEAKKFLAPKDKPSGDTAAVFEEGGDVDDLLDMI

SEQ ID NO: 35 - DNA, 3534 bp, Homo sapiens
gggcgccgggccggtgggagccagcggcgcgcggtgggacccacggagccccgcgacccgccgagcctggagccgggccgggtcggggaagccggctccagcccggagcgaacttcgcagcccgtcggggggcggcggggagggggcccggagccggaggagggggcggccgcgggcacccccgcctgtgccccggcgtccccgggcaccatgctgtccaactcccagggccagagcccgccggtgccgttccccgccccggccccgccgccgcagccccccacccctgccctgccgcaccccccggcgcagccgccgccgccgcccccgcagcagttcccgcagttccacgtcaagtccggcctgcagatcaagaagaacgccatcatcgatgactacaaggtcaccagccaggtcctggggctgggcatcaacggcaaagttttgcagatcttcaacaagaggacccaggagaaattcgccctcaaaatgcttcaggactgccccaaggcccgcagggaggtggagctgcactggcgggcctcccagtgcccgcacatcgtacggatcgtggatgtgtacgagaatctgtacgcagggaggaagtgcctgctgattgtcatggaatgtttggacggtggagaactctttagccgaatccaggatcgaggagaccaggcattcacagaaagagaagcatccgaaatcatgaagagcatcggtgaggccatccagtatctgcattcaatcaacattgcccatcgggatgtcaagcctgagaatctcttatacacctccaaaaggcccaacgccatcctgaaactcactgactttggctttgccaaggaaaccaccagccacaactctttgaccactccttgttatacaccgtactatgtggctccagaagtgctgggtccagagaagtatgacaagtcctgtgacatgtggtccctgggtgtcatcatgtacatcctgctgtgtgggtatccccccttctactccaaccacggccttgccatctctccgggcatgaagactcgcatccgaatgggccagtatgaatttcccaacccagaatggtcagaagtatcagaggaagtgaagatgctcattcggaatctgctgaaaacagagcccacccagagaatgaccatcaccgagtttatgaaccacccttggatcatgcaatcaacaaaggtccctcaaaccccactgcacaccagccgggtcctgaaggaggacaaggagcggtgggaggatgtcaaggggtgtcttcatgacaagaacagcgaccaggccacttggctgaccaggttgtgagcagaggattctgtgttcctgtccaaactcagtgctgtttcttagaatccttttattccctgggtctctaatgggaccttaaagaccatctggtatcatcttctcattttgcagaagagaaactgaggcccagaggcggagggcagtctgctcaaggtcacgcagctggtgactggttggggcagaccggacccaggtttcctgactcctggcccaagtctcttcctcctatcctgcgggatcactggggggctctcagggaacagcagcagtgccatagccaggctctctgctgcccagcgctggggtgaggctgccgttgtcagcgtggaccactaaccagcccgtcttctctctctgctcccacccctgccgccctcaccctgcccttgttgtctctgtctctcacgtctctcttctgctgtctctcctacctgtcttctggctctctctgtacccttcctggtgctgccgtgcccccaggaggagatgaccagtgccttggccacaatgcgcgttgactacgagcagatcaagataaaaaagattgaagatgcatccaaccctctgctgctgaagaggcggaagaaagctcgggccctggaggctgcggctctggcccactgagccaccgcgccctcctgcccacgggaggacaagcaataactctctacaggaatatattttttaaacgaagagacagaactgtccacatctgcctcctctcctcctcagctgcatggagcctggaactgcatcagtgactgaattctgccttggttctggccaccccagagtgggagaggctgggaggttgggaggctgtggagagaagtgagcaaggtgctcttgaacctgtgctcattttgcaattttatcagtaatttgacttagagtttttacgaaacctcttttgttgtccttgccccactcctctccaccagacgccttcctctctggatactgcaaaggcttgtggtttgttagagggtatttgtggaaactgtcatagggattgtccctgtgttgtcccatctgccctccctgtttctccacaacagcctggggttgtccccgctggctcacgcgttctgggagctcaaggccaccttggaggaggatgccacgcacttcctctctcggagccctcagacatctccagtgtgccagacaaataggagtgagtgtatgtgtgtgtgtgtgtgtgtgtgtgtgcacacgtgtgtatgagtgcgcagatctgtgcctgggatcgtgcatttgaggggccaggggcaggcagggctgcagagggagacggccctgctggggcttaggaaccttctcccttcttgggtctgccctgcccatactgagcctgccaaagtgcctgggaagcccacccagattctgaaacaggccctctgtggcctgtctctattagctgggttccgggaggcagagaggagtgaccgggcactggcactgcgatcaggaagactggacccccagcccccagggcccccctccccccacttagtgctggtcctaggtcctctgaggcactcatctactgaatgacctctctacttccccttcttgccattattaacccatttttgtttattttccttaaatttttagccatttctccatgggccaccgcccagctcatgtaggtgagcctgggcagcttctgttggcagagcttttgcatttcctgtgtttgtcctgggttctggggcatcagccagctaccccttgtgggcaaaggcagggccacttttgaagtcttccctcagatttccattgtgtggcctggtgggtcagggggagtctttgcaccaaagatgtcctgactttgcccccttgcccatcagccatttgccatcaccccaaacaactcagcttcggggccggtgaggggaggggcctcccccagcacagatgaggagcagctggggtaggctgtctgtgccatggccccccactcccccttcccttggagggagaggtggcaggaatacttcacctttcctctccctcaggggcaggtggtggaggggcgcccagggtcgtctttgtgtatgggggaaggcgctgggtgcctgcagcgcctcccttgtctcagatggtgtgtccagcactcgattgttgtaaactgttgttttgtatgagcgaaattgtctttactaaacagatttaatagttgagaaaaaaaaaaaaaaaaaa

SEQ ID NO: 36 - DNA, 2997 bp, Homo sapiens
gggcgccgggccggtgggagccagcggcgcgcggtgggacccacggagccccgcgacccgccgagcctggagccgggccgggtcggggaagccggctccagcccggagcgaacttcgcagcccgtcggggggcggcggggagggggcccggagccggaggagggggcggccgcgggcacccccgcctgtgccccggcgtccccgggcaccatgctgtccaactcccagggccagagcccgccggtgccgttccccgccccggccccgccgccgcagccccccacccctgccctgccgcaccccccggcgcagccgccgccgccgcccccgcagcagttcccgcagttccacgtcaagtccggcctgcagatcaagaagaacgccatcatcgatgactacaaggtcaccagccaggtcctggggctgggcatcaacggcaaagttttgcagatcttcaacaagaggacccaggagaaattcgccctcaaaatgcttcaggactgccccaaggcccgcagggaggtggagctgcactggcgggcctcccagtgcccgcacatcgtacggatcgtggatgtgtacgagaatctgtacgcagggaggaagtgcctgctgattgtcatggaatgtttggacggtggagaactctttagccgaatccaggatcgaggagaccaggcattcacagaaagagaagcatccgaaatcatgaagagcatcggtgaggccatccagtatctgcattcaatcaacattgcccatcgggatgtcaagcctgagaatctcttatacacctccaaaaggcccaacgccatcctgaaactcactgactttggctttgccaaggaaaccaccagccacaactctttgaccactccttgttatacaccgtactatgtggctccagaagtgctgggtccagagaagtatgacaagtcctgtgacatgtggtccctgggtgtcatcatgtacatcctgctgtgtgggtatccccccttctactccaaccacggccttgccatctctccgggcatgaagactcgcatccgaatgggccagtatgaatttcccaacccagaatggtcagaagtatcagaggaagtgaagatgctcattcggaatctgctgaaaacagagcccacccagagaatgaccatcaccgagtttatgaaccacccttggatcatgcaatcaacaaaggtccctcaaaccccactgcacaccagccgggtcctgaaggaggacaaggagcggtgggaggatgtcaaggaggagatgaccagtgccttggccacaatgcgcgttgactacgagcagatcaagataaaaaagattgaagatgcatccaaccctctgctgctgaagaggcggaagaaagctcgggccctggaggctgcggctctggcccactgagccaccgcgccctcctgcccacgggaggacaagcaataactctctacaggaatatattttttaaacgaagagacagaactgtccacatctgcctcctctcctcctcagctgcatggagcctggaactgcatcagtgactgaattctgccttggttctggccaccccagagtgggagaggctgggaggttgggaggctgtggagagaagtgagcaaggtgctcttgaacctgtgctcattttgcaattttatcagtaatttgacttagagtttttacgaaacctcttttgttgtccttgccccactcctctccaccagacgccttcctctctggatactgcaaaggcttgtggtttgttagagggtatttgtggaaactgtcatagggattgtccctgtgttgtcccatctgccctccctgtttctccacaacagcctggggttgtccccgctggctcacgcgttctgggagctcaaggccaccttggaggaggatgccacgcacttcctctctcggagccctcagacatctccagtgtgccagacaaataggagtgagtgtatgtgtgtgtgtgtgtgtgtgtgtgtgcacacgtgtgtatgagtgcgcagatctgtgcctgggatcgtgcatttgaggggccaggggcaggcagggctgcagagggagacggccctgctggggcttaggaaccttctcccttcttgggtctgccctgcccatactgagcctgccaaagtgcctgggaagcccacccagattctgaaacaggccctctgtggcctgtctctattagctgggttccgggaggcagagaggagtgaccgggcactggcactgcgatcaggaagactggacccccagcccccagggcccccctccccccacttagtgctggtcctaggtcctctgaggcactcatctactgaatgacctctctacttccccttcttgccattattaacccatttttgtttattttccttaaatttttagccatttctccatgggccaccgcccagctcatgtaggtgagcctgggcagcttctgttggcagagcttttgcatttcctgtgtttgtcctgggttctggggcatcagccagctaccccttgtgggcaaaggcagggccacttttgaagtcttccctcagatttccattgtgtggcctggtgggtcagggggagtctttgcaccaaagatgtcctgactttgcccccttgcccatcagccatttgccatcaccccaaacaactcagcttcggggccggtgaggggaggggcctcccccagcacagatgaggagcagctggggtaggctgtctgtgccatggccccccactcccccttcccttggagggagaggtggcaggaatacttcacctttcctctccctcaggggcaggtggtggaggggcgcccagggtcgtctttgtgtatgggggaaggcgctgggtgcctgcagcgcctcccttgtctcagatggtgtgtccagcactcgattgttgtaaactgttgttttgtatgagcgaaattgtctttactaaacagatttaatagttgagaaaaaaaaaaaaaaaaaa

SEQ ID NO: 37 - protein, 370 aa, Homo sapiens
MLSNSQGQSPPVPFPAPAPPPQPPTPALPHPPAQPPPPPPQQFPQFHVKSGLQIKKNAIIDDYKVTSQVLGLGINGKVLQIFNKRTQEKFALKMLQDCPKARREVELHWRASQCPHIVRIVDVYENLYAGRKCLLIVMECLDGGELFSRIQDRGDQAFTEREASEIMKSIGEAIQYLHSINIAHRDVKPENLLYTSKRPNAILKLTDFGFAKETTSHNSLTTPCYTPYYVAPEVLGPEKYDKSCDMWSLGVIMYILLCGYPPFYSNHGLAISPGMKTRIRMGQYEFPNPEWSEVSEEVKMLIRNLLKTEPTQRMTITEFMNHPWIMQSTKVPQTPLHTSRVLKEDKERWEDVKGCLHDKNSDQATWLTRL

SEQ ID NO: 38 - protein, 400 aa, Homo sapiens
MLSNSQGQSPPVPFPAPAPPPQPPTPALPHPPAQPPPPPPQQFPQFHVKSGLQIKKNAIIDDYKVTSQVLGLGINGKVLQIFNKRTQEKFALKMLQDCPKARREVELHWRASQCPHIVRIVDVYENLYAGRKCLLIVMECLDGGELFSRIQDRGDQAFTEREASEIMKSIGEAIQYLHSINIAHRDVKPENLLYTSKRPNAILKLTDFGFAKETTSHNSLTTPCYTPYYVAPEVLGPEKYDKSCDMWSLGVIMYILLCGYPPFYSNHGLAISPGMKTRIRMGQYEFPNPEWSEVSEEVKMLIRNLLKTEPTQRMTITEFMNHPWIMQSTKVPQTPLHTSRVLKEDKERWEDVKEEMTSALATMRVDYEQIKIKKIEDASNPLLLKRRKKARALEAAALAH

SEQ ID NO: 39 - DNA, 4639 bp, Homo sapiens
gagcgcgcacgtcccggagcccatgccgaccgcaggcgccgtatccgcgctcgtctagcagccccggttacgcggttgcacgtcggccccagccctgaggagccggaccgatgtggaaactgctgcccgccgcgggcccggcaggaggagaaccatacagacttttgactggcgttgagtacgttgttggaaggaaaaactgtgccattctgattgaaaatgatcagtcgatcagccgaaatcatgctgtgttaactgctaacttttctgtaaccaacctgagtcaaacagatgaaatccctgtattgacattaaaagataattctaagtatggtacctttgttaatgaggaaaaaatgcagaatggcttttcccgaactttgaagtcgggggatggtattacttttggagtgtttggaagtaaattcagaatagagtatgagcctttggttgcatgctcttcttgtttagatgtctctgggaaaactgctttaaatcaagctatattgcaacttggaggatttactgtaaacaattggacagaagaatgcactcaccttgtcatggtatcagtgaaagttaccattaaaacaatatgtgcactcatttgtggacgtccaattgtaaagccagaatattttactgaattcctgaaagcagttgagtccaagaagcagcctccacaaattgaaagtttttacccacctcttgatgaaccatctattggaagtaaaaatgttgatctgtcaggacggcaggaaagaaaacaaatcttcaaagggaaaacatttatatttttgaatgccaaacagcataagaaattgagttccgcagttgtctttggaggtggggaagctaggttgataacagaagagaatgaagaagaacataatttctttttggctccgggaacgtgtgttgttgatacaggaataacaaactcacagaccttaattcctgactgtcagaagaaatggattcagtcaataatggatatgctccaaaggcaaggtcttagacctattcctgaagcagaaattggattggcggtgattttcatgactacaaagaattactgtgatcctcagggccatcccagtacaggattaaagacaacaactccaggaccaagcctttcacaaggcgtgtcagttgatgaaaaactaatgccaagcgccccagtgaacactacaacatacgtagctgacacagaatcagagcaagcagatacatgggatttgagtgaaaggccaaaagaaatcaaagtctccaaaatggaacaaaaattcagaatgctttcacaagatgcacccactgtaaaggagtcctgcaaaacaagctctaataataatagtatggtatcaaatactttggctaagatgagaatcccaaactatcagctttcaccaactaaattgccaagtataaataaaagtaaagatagggcttctcagcagcagcagaccaactccatcagaaactactttcagccgtctaccaaaaaaagggaaagggatgaagaaaatcaagaaatgtcttcatgcaaatcagcaagaatagaaacgtcttgttctcttttagaacaaacacaacctgctacaccctcattgtggaaaaataaggagcagcatctatctgagaatgagcctgtggacacaaactcagacaataacttatttacagatacagatttaaaatctattgtgaaaaattctgccagtaaatctcatgctgcagaaaagctaagatcaaataaaaaaagggaaatggatgatgtggccatagaagatgaagtattggaacagttattcaaggacacaaaaccagagttagaaattgatgtgaaagttcaaaaacaggaggaagatgtcaatgttagaaaaaggccaaggatggatatagaaacaaatgacactttcagtgatgaagcagtaccagaaagtagcaaaatatctcaagaaaatgaaattgggaagaaacgtgaactcaaggaagactcactatggtcagctaaagaaatatctaacaatgacaaacttcaggatgatagtgagatgcttccaaaaaagctgttattgactgaatttagatcactggtgattaaaaactctacttccagaaatccatctggcataaatgatgattatggtcaactaaaaaatttcaagaaattcaaaaaggtcacatatcctggagcaggaaaacttccacacatcattggaggatcagatctaatagctcatcatgctcgaaagaatacagaactagaagagtggctaaggcaggaaatggaggtacaaaatcaacatgcaaaagaagagtctcttgctgatgatctttttagatacaatccttatttaaaaaggagaagataactgaggattttaaaaagaagccatggaaaaacttcctagtaagcatctacttcaggccaacaaggttatatgaatatatagtgtatagaagcgatttaagttacaatgttttatggcctaaatttattaaataaaatgcacaaaactttgattcttttgtatgtaacaattgtttgttctgttttcaggctttgtcattgcatctttttttcatttttaaatgtgttttgtttattaaatagttaatatagtcacagttcaaaattctaaatgtacgtaaggtaaagactaaagtcacccttccaccattgtcctagctacttggttcccctcagaaaaaaattcatgatactcatttcttatgaatctttccagggatttttgagtcctattcaaattcctatttttaaataatttcctacacaaatgatagcataacatatgcagtgttctacaccttgcttttttacttagtagattaaaaattataggaatatcaatataatgtttttaatattttttcttttccattatgctgtagtcttacctaaactctggtgatccaaacaaaatggcttcagtggtgcagatgtcacctacatgttattctagtactagaaactgaagaccatgtggagacttcatcaaacatgggtttagttttcaccagaatggaaagacctgtacccctttttggtggtcttactgagctgggtgggtgtctgttttgagcttatttagagtcctagttttcctacttataaagtagaaatggtgagattgttttctttttctaccttaaagggagatggtaagaaacaatgaatgtcttttttcaaactttattgacaagtgattttcaagtctgtgttcaaaaatatattcatgtacctgtgatccagcaagaagggagttccagtcaagagtcactacaactgattagttgtttagagaatgagaaatggaacagtgaggaatggaggccatatttccatgacttcccttgtaaacagaagcaacagaagggacaagaggctggcctctacatcactctcaccttccaaatcttgtggaagtgcatctacttgccagaaccaaattaacttacttccaagttctggctgcttgcaggtggaactccagctgcaagggagttagggaaatgaaggtctttttttaaaagcttctcagccttcctagggaacagaaattgggtgagccaatctgcaatttctactacaggcattgagaccagttagattattgaaatattatagagagttatgaacacttaaattatgatagtggtatgacattggatagaacatgggatactttagaagtagaattgacagggcatattagttgatgaaatggagtcatttgagtctcttaatagccatgtatcataattaccaagtgaagctggtggaacatatggtctccattttacagttaaggaatataatggacagattaatattgttctctgtcatgcccacaatccctttctaaggaagactgccctactatagcagtttttatatttgtcaatttatgaatataatgaatgagagttctggtacctcctgtctttacaaatattggtgttgtcagtatttttcctttttaaccattccaatcggtgtgtagtgatgtttcattttggttttaatttgtatatccctgatagctataattgggtcatagaaattctttatacattctagatgcaagtctcttgtcggatatatgtattgagatattacacctagtctgtggcttgactgttttctttatgtcttttgatgaatagaagttttaaattttgacaaggtcaaatttatttttttcttttgtttgatattttttctctccaatttaaccccaagatttcagatattctgctctattatataaactttatatttttatatttgtgatctaccttgaattgatatgtatgttgtgaattatggatcagggttctttttttcccccatacaagtatccagtcattgtaacactgtttattgaaagaattatcctttcctcattaaattaccttgccaattagtaaaaaatcaattaaccataatggtggatctgtttctggactttctgtttggttacactgaaatgtttgtccatccttgcactcactcataccatactgccttgaattactgtagctgcatagatgctccttaagttgggattacattgtaataaacgcaatgtaagttaaaaaaaaaaaaaaaaaa

SEQ ID NO: 40 - protein, 754 aa, Homo sapiens
MWKLLPAAGPAGGEPYRLLTGVEYVVGRKNCAILIENDQSISRNHAVLTANFSVTNLSQTDEIPVLTLKDNSKYGTFVNEEKMQNGFSRTLKSGDGITFGVFGSKFRIEYEPLVACSSCLDVSGKTALNQAILQLGGFTVNNWTEECTHLVMVSVKVTIKTICALICGRPIVKPEYFTEFLKAVESKKQPPQIESFYPPLDEPSIGSKNVDLSGRQERKQIFKGKTFIFLNAKQHKKLSSAVVFGGGEARLITEENEEEHNFFLAPGTCVVDTGITNSQTLIPDCQKKWIQSIMDMLQRQGLRPIPEAEIGLAVIFMTTKNYCDPQGHPSTGLKTTTPGPSLSQGVSVDEKLMPSAPVNTTTYVADTESEQADTWDLSERPKEIKVSKMEQKFRMLSQDAPTVKESCKTSSNNNSMVSNTLAKMRIPNYQLSPTKLPSINKSKDRASQQQQTNSIRNYFQPSTKKRERDEENQEMSSCKSARIETSCSLLEQTQPATPSLWKNKEQHLSENEPVDTNSDNNLFTDTDLKSIVKNSASKSHAAEKLRSNKKREMDDVAIEDEVLEQLFKDTKPELEIDVKVQKQEEDVNVRKRPRMDIETNDTFSDEAVPESSKISQENEIGKKRELKEDSLWSAKEISNNDKLQDDSEMLPKKLLLTEFRSLVIKNSTSRNPSGINDDYGQLKNFKKFKKVTYPGAGKLPHIIGGSDLIAHHARKNTELEEWLRQEMEVQNQHAKEESLADDLFRYNPYLKRRR

SEQ ID NO: 41 - DNA, 1491 bp, Homo sapiens
cactcagaaaggccgctgggtgcggggagcgcagaggcggtgcagggcggctggctcgcctcggcgtgcagtgcgcgtgcgtggagctgggagctaggtcctcggagtgggccagagatggcggcggccgacggggctttgccggaggcggcggctttagagcaacccgcggagctgcctgcctcggtgcgggcgagtatcgagcggaagcggcagcgggcactgatgctgcgccaggcccggctggctgcccggccctactcggcgacggcggctgcggctactggaggcatggctaatgtaaaagcagccccaaagataattgacacaggaggaggcttcattttagaagaggaagaagaagaagaacagaaaattggaaaagttgttcatcaaccaggacctgttatggaatttgattatgtaatatgcgaagaatgtgggaaagaatttatggattcttatcttatgaaccactttgatttgccaacttgtgataactgcagagatgctgatgataaacacaagcttataaccaaaacagaggcaaaacaagaatatcttctgaaagactgtgatttagaaaaaagagagccacctcttaaatttattgtgaagaagaatccacatcattcacaatggggtgatatgaaactctacttaaagttacagattgtgaagaggtctcttgaagtttggggtagtcaagaagcattagaagaagcaaaggaagtccgacaggaaaaccgagaaaaaatgaaacagaagaaatttgataaaaaagtaaaagaattgcggcgagcagtaagaagcagcgtgtggaaaagggagacgattgttcatcaacatgagtatggaccagaagaaaacctagaagatgacatgtaccgtaagacttgtactatgtgtggccatgaactgacatatgaaaaaatgtgattttttagttcagtgacctgttttatagaattttatatttaaataaaggaaatttagattggtccttttcaaaattcaaaaaaaaaagcaacatcttcatagatgaatgaaacccttgtataagtaatacttcagtaataattatgtatgttatggcttaaaagcaagtttcagtgaaggtcacctggcctggttgtgtgcacaatgtcatgtctgtgattgccttcttacaacagagatgggagctgagtgctagagtaggtgcagaagtggtaggtcagctacaaatttgaggacaagataccaaggcaaaccctagattggggtagagggaaaagggttcaacaaaggctgaactggattcttaaccaagaaacaaataatagcaatggtggtgcaccactgtaccccaggttctagtcatgtgttttttaggacgatttctgtctccacgatggtggaaacagtggggaactactgctggaaaaagccctaatagcagaaataaacattgagttgtacgagtctgaaaaaaaaaaaaaaaaaaaaaaa

SEQ ID NO: 42 - protein, 273 aa, Homo sapiens
MAAADGALPEAAALEQPAELPASVRASIERKRQRALMLRQARLAARPYSATAAAATGGMANVKAAPKIIDTGGGFILEEEEEEEQKIGKVVHQPGPVMEFDYVICEECGKEFMDSYLMNHFDLPTCDNCRDADDKHKLITKTEAKQEYLLKDCDLEKREPPLKFIVKKNPHHSQWGDMKLYLKLQIVKRSLEVWGSQEALEEAKEVRQENREKMKQKKFDKKVKELRRAVRSSVWKRETIVHQHEYGPEENLEDDMYRKTCTMCGHELTYEKM

SEQ ID NO: 43 - DNA, 1722 bp, Homo sapiens
cactcagaaaggccgctgggtgcggggagcgcagaggcggtgcagggcggctggctcgcctcggcgtgcagtgcgcgtgcgtggagctgggagctaggtcctcggagtgggccagagatggcggcggccgacggggctttgccggaggcggcggctttagagcaacccgcggagctgcctgcctcggtgcgggcgagtatcgagcggaagcggcagcgggcactgatgctgcgccaggcccggctggctgcccggccctactcggcgacggcggctgcggctactggaggcatggctaatgtaaaagcagccccaaagataattgacacaggaggaggcttcattttagaagaggaagaagaagaagaacagaaaattggaaaagttgttcatcaaccaggacctgttatggaatttgattatgtaatatgcgaagaatgtgggaaagaatttatggattcttatcttatgaaccactttgatttgccaacttgtgataactgcagagatgctgatgataaacacaagcttataaccaaaacagaggcaaaacaagaatatcttctgaaagactgtgatttagaaaaaagagagccacctcttaaatttattgtgaagaagaatccacatcattcacaatggggtgatatgaaactctacttaaagttacagattgtgaagaggtctcttgaagtttggggtagtcaagaagcattagaagaagcaaaggaagtccgacaggaaaaccgagaaaaaatgaaacagaagaaatttgataaaaaagtaaaagaggttgttcccgccttcctgaataggccacaaagggctgagactggagtagacactgtagagattagacacaaggagcttactgtgcagttttctcagaaggctcagaacagagaggaataaaagcaagtctaaaagtattttagttgaaaagaaattggaacctggttcctcttggtcttctgattttgatccacaaagaagctgacacagtttacttctttatgggaagaattgcggcgagcagtaagaagcagcgtgtggaaaagggagacgattgttcatcaacatgagtatggaccagaagaaaacctagaagatgacatgtaccgtaagacttgtactatgtgtggccatgaactgacatatgaaaaaatgtgattttttagttcagtgacctgttttatagaattttatatttaaataaaggaaatttagattggtccttttcaaaattcaaaaaaaaaagcaacatcttcatagatgaatgaaacccttgtataagtaatacttcagtaataattatgtatgttatggcttaaaagcaagtttcagtgaaggtcacctggcctggttgtgtgcacaatgtcatgtctgtgattgccttcttacaacagagatgggagctgagtgctagagtaggtgcagaagtggtaggtcagctacaaatttgaggacaagataccaaggcaaaccctagattggggtagagggaaaagggttcaacaaaggctgaactggattcttaaccaagaaacaaataatagcaatggtggtgcaccactgtaccccaggttctagtcatgtgttttttaggacgatttctgtctccacgatggtggaaacagtggggaactactgctggaaaaagccctaatagcagaaataaacattgagttgtacgagtctgaaaaaaaaaaaaaaaaaaaaaaa